SEQUENCE LISTING (91 sequences)

SEQ ID NO: 1 - protein, 857 aa, Leishmania major
MGKRKGNSLGDSGSAATASREASAQAEDAASQTKTASPPAKVILLPKTLTDEKDFIGIFPFPFWPVHFVLTVVALFVLAASCFQAFTVRMISVQIYGYLIHEFDPWFNYRAAEYMSTHGWSAFFSWFDYMSWYPLGRPVGSTTYPGLQLTAVAIHRALAAAGMPMSLNNVCVLMPAWFGAIATATLAFCTYEASGSTVAAAAAALSFSIIPAHLMRSMAGEFDNECIAVAAMLLTFYCWVRSLRTRSSWPIGVLTGVAYGYMAAAWGGYIFVLNMVAMHAGISSMVDWARNTYNPSLLRAYTLFYVVGTAIAVCVPPVGMSPFKSLEQLGALLVLVFLCGLQVCEVLRARAGVEVRSRANFKIRVRVFSVMAGVAALAISVLAPTGYFGPLSVRVRALFVEHTRTGNPLVDSVAEHQPASPEAMWAFLHVCGVTWGLGSIVLAVSTFVHYSPSKVFWLLNSGAVYYFSTRMARLLLLSGPAACLSTGIFVGTILEAAVQLSFWDSDATKAKKQQKQAQRHQRGAGKGSGRDDAKNATTARAFCDVFAGSSLAWGHRMVLSIAMWALVTTTAVSFFSSEFASHSTKFAEQSSNPMIVFAAVVQNRATGKPMNLLVDDYLKAYEWLRDSTPEDARVLAWWDYGYQITGIGNRTSLADGNTWNHEHIATIGKMLTSPVVEAHSLVRHMADYVLIWAGQSGDLMKSPHMARIGNSVYHDICPDDPLCQQFGFHRNDYSRPTPMMRASLLYNLHEAGKRKGVKVNPSLFQEVYSSKYGLVRIFKVMNVSAESKKWVADPANRVCHPPGSWICPGQYPPAKEIQEMLAHRVPFDQVTNADRKNNVGSYQEEYMRRMRESENRR

SEQ ID NO: 2 - DNA, 2575 bp, Leishmania major
aatgggcaagcgcaagggcaacagcctcggcgacagcggcagcgccgccaccgcctcacgagaggcctctgcccaggccgaggacgccgccagccagaccaagaccgccagcccccctgccaaggtcatcctcctgcccaagaccctcaccgacgagaaggacttcatcggcatcttcccgttcccgttctggcccgtccacttcgtcctcaccgtcgtcgccctcttcgtcctcgccgccagctgcttccaggccttcaccgtccgcatgatcagcgtccagatctacggctacctcatccacgagttcgacccctggttcaactaccgagccgccgagtacatgagcacccacggctggtccgcctttttcagctggttcgactacatgagctggtatccgctcggccgacccgtcggcagcaccacctaccccggcctccagctcaccgccgtggccatccatcgagccctcgccgctgccggcatgcctatgagcctcaacaacgtctgcgtcctcatgcccgcctggttcggcgccattgcgaccgccaccctcgcgttctgcacctacgaggccagcggctctacagtggccgctgccgcggctgccctcagcttcagcatcatccccgcccacctcatgcgctccatggccggcgagttcgacaacgagtgcattgccgtcgccgccatgctcctcaccttctactgctgggtccgcagcctccgcacgcgcagcagctggcccatcggcgtcctgaccggcgtcgcctacggctacatggctgccgcctggggcggctacatcttcgtcctcaacatggtggccatgcacgccggcatcagcagcatggtcgactgggcccgcaacacctacaaccccagcctgctccgcgcctacaccctcttctacgtcgtcggcaccgccattgccgtctgcgtcccccccgtcggcatgagccccttcaagagcctcgagcagctcggcgccctcctcgtcctggtctttctgtgcggcctccaggtctgcgaggtcctccgagcccgagccggcgtcgaggtccgctctcgcgccaacttcaagatccgcgtccgcgtctttagcgtcatggccggcgtggccgccctcgccatctctgtcctcgcccccaccggctacttcggccccctcagcgtccgagtgcgcgccctgttcgtcgagcacacccgcaccggcaaccccctcgtcgacagcgtcgccgagcaccagcccgccagccccgaggccatgtgggcctttctccacgtctgcggcgtcacctggggcctcggcagcatcgtcctggccgtcagcaccttcgtccactacagccccagcaaggtcttttggctcctcaactctggcgccgtctactacttctcgacccgaatggcccgcctcctcctcctgtccggccctgccgcctgcctgagcaccggcatcttcgtcggcacgatcctcgaggccgccgtccagctcagcttctgggacagcgacgccaccaaggccaagaagcagcagaagcaggcccagcgccaccagcgaggcgctggcaagggctctggccgcgacgacgccaagaacgcgacgaccgcccgagccttctgcgacgtctttgccggcagcagcctcgcctggggccaccgcatggtcctctcgatcgccatgtgggcgctcgtcacgacaacggccgtcagcttcttcagcagcgagttcgccagccacagcaccaagttcgccgagcagagcagcaaccccatgatcgtctttgccgccgtcgtccagaaccgcgccaccggcaagccgatgaacctcctcgtcgacgactacctcaaggcctacgagtggctccgcgacagcacccctgaggacgcccgcgtcctggcctggtgggactacggctaccagatcaccggcatcggcaaccgcaccagcctcgccgacggcaacacctggaaccacgagcacattgccaccatcggcaagatgctcaccagcccggtcgtcgaggcccacagcctcgtccgccacatggccgactacgtcctcatctgggctggccagagcggcgacctcatgaagtccccccacatggcccgcatcggcaacagcgtctaccacgacatctgccccgacgaccccctctgccagcagttcggcttccaccgcaacgactacagccgccccaccccgatgatgcgcgccagcctcctctacaacctccacgaggccggcaagcgaaagggcgtcaaggtcaacccctcgctgttccaggaggtctacagcagcaagtacggcctggtccgcatcttcaaggtcatgaacgtcagcgccgagagcaagaagtgggtcgccgatcccgccaaccgagtctgccacccccctggcagctggatctgccctggccagtaccctcccgccaaggaaatccaggagatgctcgcccaccgcgtcccgttcgaccaggtcaccaacgccgaccgcaagaacaacgtcggcagctaccaagaggagtacatgcgccgcatgcgcgagagcgagaaccgccgctag

SEQ ID NO: 3 - DNA, 50 bp, Trichoderma reesei
accaaagactttttgatcaatccaacaacttctctcaacttaattaaatc

SEQ ID NO: 4 - DNA, 48 bp, Aspergillus niger
ttaattaagatccacttaacgttactgaaatcatcaaacagcttgacg

SEQ ID NO: 5 - DNA, 1000 bp, Trichoderma reesei
caagtcttcgtactctatcgaagtctcgccttacgtacttgatctgctgtctttcgtgtccggtcaacatatactcgcacacattagccccagcagaacatgtcgtcggcataaaaggccaattcagatcgcagataacaaaatgctaccagcatctgtctagttgtggagatatgaaggggtatttcaggctttctttgtgggaataaagagagaaagagagacttacaggagctctaggcttcgtagcccctgcgttcttagttcgcaatgccgtgaaagcagctacatctaccaagacactcgtgcatcgtctattttatttgttacatgctgggaatttccgggacattgtttaaggatgactaggttcagccgttaaagaatggaaggccatggcttgtccctctgtggcaagtcattgcactccaaggccttctcctgtactagtcctacaattctgcagcaaatggcctcaagcaactacgtaaaactccatgagattgcagatgcggcccactggaatacaacatcctccgcaagtccgacatgaagccccttgacttgattggcaggctaaatgcgacatcttagccggatgcaccccagatctggggaacgcgccgcttgaggcccgaagcgccgggttcgatgcattactgccatatttcagcagttaactaggaccggcttgtgtcgatattgcgggtggcgttcaatctattccggcactcctatgccgtttgatccgatacctggagggcgtgctttaggcaaaatgccaagcttcgaggatactgtacgagccgctttcaacctcacttgatgatgtctgagtttcatcaagagaattgaagtcaaagctcaaatcatgatgtgaagaggttttgaatgtggaagaattctgcatatataaagccatggaagaagacgtaaaactgagacagcaagctcaactgcatagtatcgacttcaaggaaaacacgcacaaataatcatc

SEQ ID NO: 6 - DNA, 1000 bp, Trichoderma reesei
aggggtttgagctggtatgtagtattggggtggttagtgagttaacttgacagactgcactttggcaacagagccgacgattaagagattgctgtcatgtaactaaagtagcctgcctttgacgctgtatgctcatgatacatgcgtgacatcgaaatatatcagccaaagtatccgtccggcgacatgcccatcaactatattgaagtcagaaacacactgtccctcttccctcctatgcttttacaagctgctcctctatccgcccccacagtcccttgttcatataccccgaaagccaaaagtttccatccttgtccttgcccatgatcgggaagccgtttggtagcacgataccccactgattattctgtatatagatcggtgaacccgatttcccaccctccctactgggctgaagcacagctgcagaaaagtccaagtcgaacagctttgccttgccccaatttgacaacgtaatcatgtgcatgttgccgttgccgaagaaaggcggaatcctcccgctagatcctcgccacatagcgaaaaaggcttctacctgagaccgagttcccagttcttgaatcgcggttcgagtagcagcagcaatataactcagcggcttctcaaatatgtggtgcaccggcagtagcacgttgatgaagccggtaccgttggagacatatggcacccctttcggcagcagatccgtctctagacactttcgtagagagtatgcgttgttgatgacaaccgtcctctggctattcgctggcagatgtgaagtggcaactttgatccaccaggcgcagagaacatcgccttcagtcaagaaagtgttttctgcgccctcggactcaagctcactgattgcctctttgcgaaggttctcaatgaaagatccaggaacacaaagcatgcgattctcttgcgctcggaagagatcgaggacattgttgatcccatactgggccagcccaaacattgacaagcgccgaga

SEQ ID NO: 7 - protein, 688 aa, Trichomonas vaginalis
MGNTVKVIQLITLLLSCLLAFLIRQFANVVNEPIIHEFDPHFNWRCTQYIDTHGLYEFLGWFDNISWYPQGRPVGETAYPGLMYTSAIVKWALQKIHIIVDLRNICVFMGPSVSILSVLVAFLFGELVGSAQLGTLFGAITSFIPGMISRSVGGAYDYECIGLFIIVLSLYTFALALKSGSILLSVIAAFAYSYLALTWGGYVFVSNCIPLFAAGLVAIGRYSWRLHITYSIWFIVASILTAQIPFIGDKILKKPEHFAMLGTFLVMQIWGFFTFIKSRFSPTTYNSVAITSILILPSFLLLMITVGMSTGLLGGFSGRLLQMFDPTYAAKNVPIINSVAEHQPTAWVKYYSDCELFIFFFPLGAYIVISSLIRTQKTKDQTELKRAETLLLLFIYGFSTLYFASIMVRLVLVFTPALVFVAGIAIHQLLRESFKQKSFLHPVSLTMIILTFIICLHGVLHATHFACYSYSGDHLHFNIMTPRGVETSDDYREGYRWLTENTYRDDIVMSWWDYGYQITSMGNRGCIADGNTNNFTHIGIIGMAMSSPEPISWRIARLMNVKYMLVIFGGAAQYSGDDINKFLWMPRIAHQTFDNITGEMYQIPYRHIVGESMTKNMTLSMMFKFCYNNYKYYQPHPQFPTGYDLTRRTSIPNIKDISMSQFTEAFTTKNWIVRIYKVGDDPQWNRVY

SEQ ID NO: 8 - protein, 836 aa, Leishmania infantum
MGKKGNLLGDSGSAATASPPANMILLPKTPIDTKDFIGIFSFPFWPVRFVVTVVALFVVGASCFQAFTVRMTSVQIYGYLIHEFDPWFNYRAAEYMSTHGWSAFFSWFDYMSWYPLGRPVGSTTYPGLQLTAVAIHRALAAAGMPMSLNNVCVLMPAWFGAIATATLAFCTYEASGSTVAAAAAALSFSIIPAHLMRSMAGEFDNECIAVAAMLLTFYCWVRSLRTRSSWPIGVLTGVAYGYMVAAWGGYIFVLNMVAMHAGISSMVDWARNTYNPSLLRAYTLFYVVGTAIAVCVPPVGMSPFKSLEQLGALLVLVFLCGLQACEVFRARAGVEVRSRANFKIRVRVFSVMAGVAALAIAVLAPTGYFGPLSVRVRALFVEHTRTGNPLVDSVAEHQPAGPEAMWSFLHVCGVTWGLGSIVLALSTFVHYAPSKLFWLLNSGAVYYFSTRMARLLLLSGPAACLSTGIFVGTILEAAVQLSFWDSDATKARKQQKPAQRHRRGAGKDSDRDDAESATTARTLCDVFAGSPLAWGHRMVLFIAVWALVTTTAVSFFSSDFASHSTTFAEQSSNPMIVFAAVVQNRATGKPMNILVDDYLRSYIWLRDNTPEDARILAWWDYGYQITGIGNRTSLADGNTWNHEHIATIGKMLTSPVAEAHSLVRHMADYVLIWAGQSGDLMKSPHMARIGNSVYHDICPHDPLCQQFGFYRNDYSRPTPMMRASLLYNLHEVGKTKGVKVDPSLFQEVYSSKYGLVRVFKVMNVSEESKKWVADPANRVCHPPGSWICPGQYPPAKEIQEMLAHRVPFDQVEKVDRKNHVGSYHEEYMRRMRESES

SEQ ID NO: 9 - DNA, 2511 bp, Leishmania infantum
atgggcaagaagggcaacctcctcggcgatagcggctctgctgccaccgccagcccccctgccaacatgatcctgctccccaagacccccatcgacaccaaggacttcatcggcatcttcagcttcccgttctggcccgtccgcttcgtcgtcaccgtcgtcgccctcttcgtcgtcggcgccagctgcttccaggccttcaccgtccgcatgaccagcgtccagatctacggctacctcatccacgagttcgacccctggttcaactaccgagccgccgagtacatgagcacccacggctggtccgcctttttcagctggttcgactatatgagctggtatcccctcggccgacccgtcggcagcaccacctaccccggcctccagctcaccgctgtcgccatccaccgagccctcgctgcggctggcatgcccatgagcctcaacaacgtctgcgtcctcatgcccgcctggttcggcgccattgcgaccgccaccctcgcgttctgcacctacgaggccagcggcagcacagtggctgctgccgctgcggccctcagcttcagcatcatccccgcccacctcatgcgcagcatggccggcgagttcgacaacgagtgcattgccgtcgccgccatgctcctcaccttctactgctgggtccgctccctccgcacccgcagcagctggcccatcggcgtcctcaccggggtcgcctacggctacatggtggccgcctggggcggctacatcttcgtcctcaacatggtcgccatgcacgccggcatcagcagcatggtcgactgggcccgcaacacctacaaccccagcctgctccgcgcctacaccctcttctacgtcgtcggcaccgccattgccgtctgcgtcccccccgtcggcatgagccccttcaagagcctcgagcagctcggagcgctgctcgtcctggtctttctgtgcggcctccaggcctgcgaggtctttcgcgcccgagccggcgtcgaggtccgcagccgcgccaacttcaagatccgcgtccgcgtgttcagcgtcatggccggcgtcgccgccttggctatcgccgtcctcgcccccaccggctacttcggccccctcagcgtccgcgtgcgcgccctgttcgtcgagcacacccgcaccggcaatcccctggtcgacagcgtcgccgagcaccagcctgccggccctgaggccatgtggtcgttcctccacgtctgcggcgtcacctggggcctcggatccatcgtcctggccctcagcaccttcgtccactacgcccccagcaagctgttctggctcctcaactctggcgccgtctactacttctcgacccgaatggcccgcctcctgctcctcagcggccctgccgcctgcctcagcaccggcatcttcgtgggcaccatcctcgaggccgccgtccagctcagcttctgggacagcgacgccaccaaggcccgcaagcagcagaagcctgcccagcgccaccgacggggagccggcaaggatagcgaccgcgacgacgccgagtctgccaccaccgcccgcaccctctgcgacgtctttgccggcagccccctcgcctggggccaccgcatggtcctcttcattgccgtgtgggccctcgtcacgacgaccgccgtcagcttcttcagcagcgacttcgccagccacagcaccaccttcgccgagcagagcagcaaccccatgatcgtctttgccgccgtcgtccagaaccgcgccaccggcaagccgatgaacatcctcgtcgacgactacctccgcagctacatctggctccgcgacaacacccccgaggacgcccgcatcctcgcctggtgggactacggctaccagatcaccggcatcggcaaccgcaccagcctcgccgacggcaacacctggaaccacgagcacattgccaccatcggcaagatgctcaccagccccgtcgccgaggcccacagcctcgtccgccacatggccgactacgtcctcatctgggctggccagagcggcgacctcatgaagtccccccacatggcccgcatcggcaacagcgtctaccacgacatctgcccccacgaccccctctgccagcagttcggcttctaccgcaacgactacagccgccccaccccgatgatgcgcgccagcctcctctacaacctccacgaggtcggcaagaccaagggcgtcaaggtcgaccccagcctcttccaagaggtctacagcagcaagtacggcctcgtgcgcgtgttcaaggtcatgaacgtcagcgaagagtccaagaagtgggtcgcggaccccgccaacagggtctgccacccccctggcagctggatctgccctggccagtaccctcccgccaaagagatccaagagatgctcgcccaccgcgtcccgttcgaccaggtcgagaaggtcgaccgcaagaaccacgtcggctcctaccacgaagagtacatgcgccgcatgcgcgagagcgagagctga

SEQ ID NO: 10 - protein, 721 aa, Entamoeba histolytica
MGFFKTLVQLILKNIGITLICIIAFSSRLYSIIMYEAIIHEFDPYFNFRATKYLVEHGPTAFMNWFDPDSWYPLGRNIGTTVFPGLMFTSAFIFKFLAYFNLIIDVRLICVCMGPIYSVITCIVAYLFGSRVHSDRAGLFAAALISVVPGYMSRSVAGSYDYECISITILILTFYLWIEAVHNNSPILSAVTALSYFYMASTWGAYVFINNIIPLHVLISIFCGFYNKKLYSCYSIYYIFATILSMQVPFINYVPIRSSEHIGAMGVFGICQLIELYSLIHKLLGQKKTVELIKKVLMGSVIIGIIMVLILIKKGYISAWSGRFYALFDPTFAKKNIPLIVSVSEHQPANWASYFFDLHCLIVIAPAGLYYCFKKFDFNMLFLIIYSVSVFYFSCVMSRLVLILAPAICLLSGIALAEFFTQIQKQLESTLKMVFKSNKKQQQQQSNEPTTKIEKEKRKIHPPKKEQNNEKSFISEFIIFIIMTIVGILLIIFLFKFFEYSIQMSKNYSSPSVVLYGNHGGKQIAFDDYREAYRWLAHNTPEGSRVMSWWDYGYQISHLANRTVIVDNNTWNNSHIALTGNVMASREEDAMKTIRDLDVDYLLVVFGGYLGYSSDDINKFLWMIRIGAGVNPSLNENNYYNHNAYTVADPSDTFKYSMMYKMCYHNFYKASNGYRAGMDAVRREVIEEQTYFKNIQEAFTSQHWVVRIYKVNKPNPIDSLL

SEQ ID NO: 11 - DNA, 40 bp, Trichoderma reesei
agctccgtggcgaaagcctgacgcaccggtagattcttgg

SEQ ID NO: 12 - DNA, 1000 bp, Artificial
  Primer
gttgggctgaggccgtatcggagggacggggtgaggattgaggcggaggagatgaagggggatgatggggagacggtggttgttgtgcataattatgggcatgcgggatgggggtatcaggggtcgtatgggtgtgcggagagggttgtcgagttggtggaggggattgtgaggggatgagcggatgtttttgatgttttgactgctcgcctttgactcgattctgatacggacacttttcgacctttgtttctccaagatggccctgtacagtcagattgatagaggagcatgtataattcattgccggttgccgtcccgtttccaagcagaaagccactgttgagaagcaacgtgctttgacgaaagtcgtggctcactactcaaatctctccacactcatacattgtgtttcagtcaaaacactttggcaaccaagacgtgggagggagtatctgcatcttttctcatcggcaagctatctgactcgattgagaagatgcgtggttcatatcacctggccgttggaggtttcttcctaggcagtcgctctgttctccttctataaagaactccatcgttcttgaatacctctttggccttcaagctcgatagtattgaacccattcttcactcatgctgctcatcattccacctccctcaagttgggtgtcgttgagtacctagtgtacataagcgggtctatgcatttaaaggggtatcttcaccaccagcaatatccacacttctaggctccacgttgcacataacgaaaccaaaacagctaaaccgacgggccaatttcacgcgcatcttcatcgacgaagcgagcgacagcgaagccgatacgcaaatcctcttcagacaagctcaactcggccaagcctcatgttttgccaacggaaccctgcacaagtcggctggcattaaagaggaaaggagaacagaaagagagtgagcagatttcagtctctcaccactcacctgagttgcctctctc

SEQ ID NO: 13 - DNA, 1001 bp, Artificial
  Primer
gggcagtatgccggatggctggcttatacaggcaaaaaccaccttcttcattcttcattcttcgtcttcttcttcttcttcctcctcatcgtcggtaggcggcagctttcccacattggagtcgctctcctcgtcgctgagttcctcgaccgtcttttcgaattcctttggcctggagtcatcataatagtttaatacacgtttagagtatagagagaaaaaataagggggaaaaagacgcaaatcataccagtacggctgcttccgccagagcttctcgtcgcgcacgaccttgataatctcgccaaaggccctgctgtcctgcgtgccgacgggatatccctcggccaggacgtagccgccgcgcttgatccagacggtgttgcgcaggcgctgggcgagctcgaggacgacgtgcttcttgctgggcatctcgcaggtgtagaggttgttgccctcgggcttcaggacgcgcacaatggcctgcgtcggctcgagggcatcgggaggcgtgagggcctcttgtgtggcggcaaggacatttcgcttcggcttacccatggctgcgagtctttggggtcgattcggtgatactatctgatcccaagaaaaaagagacaaaatttcattgttgttgattggaaaataaactggggccgtgatggaggggcagctttatcgataggacggggatttctcgaataggaaaataaaacccctccgcccgtcccgctctccggcacggtgttgccccattcggcgaaaccgcttcagggaccaaactagaagtaaggtacctatccataagctatcacgatgatatagaaggcatggatgtattgcaaaagcgaattgttagacgccccaatgggaggcttggtggggttatcggtttacgaaatacttgaatcaatgcattattaatctatccattaggcattttggcgttcaccagaccgtttgactcaccgatatcgttcgtggtggtactcggccagatg

SEQ ID NO: 14 - DNA, 19 bp, Artificial
  Primer
gcacactttcaagattggc

SEQ ID NO: 15 - DNA, 19 bp, Artificial
  Primer
gcacactttcaagattggc

SEQ ID NO: 16 - DNA, 19 bp, Artificial
  Primer
gtacggtgttgccaagaag

SEQ ID NO: 17 - DNA, 448 bp, Artificial
  Primer
gttgagtacatcgagcgcgacagcattgtgcacaccatgcttcccctcgagtccaaggacagcatcatcgttgaggactcgtgcaacggcgagacggagaagcaggctccctggggtcttgcccgtatctctcaccgagagacgctcaactttggctccttcaacaagtacctctacaccgctgatggtggtgagggtgttgatgcctatgtcattgacaccggcaccaacatcgagcacgtcgactttgagggtcgtgccaagtggggcaagaccatccctgccggcgatgaggacgaggacggcaacggccacggcactcactgctctggtaccgttgctggtaagaagtacggtgttgccaagaaggcccacgtctacgccgtcaaggtgctccgatccaacggatccggcaccatgtctgacgtcgtcaagggcgtcgagtacg

SEQ ID NO: 18 - protein, 399 aa, Trichoderma reesei
MQPSFGSFLVTVLSASMAAGSVIPSTNANPGSFEIKRSANKAFTGRNGPLALARTYAKYGVEVPKTLVDAIQLVKSIQLAKRDSATVTATPDHDDIEYLVPVKIGTPPQTLNLDFDTGSSDLWVFSSDVDPTSSQGHDIYTPSKSTSSKKLEGASWNITYGDRSSSSGDVYHDIVSVGNLTVKSQAVESARNVSAQFTQGNNDGLVGLAFSSINTVKPTPQKTWYDNIVGSLDSPVFVADLRHDTPGSYHFGSIPSEASKAFYAPIDNSKGFWQFSTSSNISGQFNAVADTGTTLLLASDDLVKAYYAKVQGARVNVFLGGYVFNCTTQLPDFTFTVGEGNITVPGTLINYSEAGNGQCFGGIQPSGGLPFAIFGDIALKAAYVIFDSGNKQVGWAQKK

SEQ ID NO: 19 - protein, 452 aa, Trichoderma reesei
MEAILQAQAKFRLDRGLQKITAVRNKNYKRHGPKSYVYLLNRFGFEPTKPGPYFQQHRIHQRGLAHPDFKAAVGGRVTRQKVLAKKVKEDGTVDAGGSKTGEVDAEDQQNDSEYLCEVTIGTPGQKLMLDFDTGSSDLWVFSTELSKHLQENHAIFDPKKSSTFKPLKDQTWQISYGDGSSASGTCGSDTVTLGGLSIKNQTIELASKLAPQFAQGTGDGLLGLAWPQINTVQTDGRPTPANTPVANMIQQDDIPSDAQLFTAAFYSERDENAESFYTFGYIDQDLVSASGQEIAWTDVDNSQGFWMFPSTKTTINGKDISQEGNTAIADTGTTLALVSDEVCEALYKAIPGAKYDDNQQGYVFPINTDASSLPELKVSVGNTQFVIQPEDLAFAPADDSNWYGGVQSRGSNPFDILGDVFLKSVYAIFDQGNQRFGAVPKIQAKQNLQPPQ

SEQ ID NO: 20 - protein, 395 aa, Trichoderma reesei
MKSALLAAAALVGSAQAGIHKMKLQKVSLEQQLEGSSIEAHVQQLGQKYMGVRPTSRAEVMFNDKPPKVQGGHPVPVTNFMNAQYFSEITIGTPPQSFKVVLDTGSSNLWVPSQSCNSIACFLHSTYDSSSSSTYKPNGSDFEIHYGSGSLTGFISNDVVTIGDLKIKGQDFAEATSEPGLAFAFGRFDGILGLGYDTISVNGIVPPFYQMVNQKLIDEPVFAFYLGSSDEGSEAVFGGVDDAHYEGKIEYIPLRRKAYWEVDLDSIAFGDEVAELENTGAILDTGTSLNVLPSGLAELLNAEIGAKKGFGGQYTVDCSKRDSLPDITFSLAGSKYSLPASDYIIEMSGNCISSFQGMDFPEPVGPLVILGDAFLRRYYSVYDLGRDAVGLAKAK

SEQ ID NO: 21 - protein, 426 aa, Trichoderma reesei
MKFHAAALTLACLASSASAGVAQPRADEVESAEQGKTFSLEQIPNERYKGNIPAAYISALAKYSPTIPDKIKHAIEINPDLHRKFSKLINAGNMTGTAVASPPPGADAEYVLPVKIGTPPQTLPLNLDTGSSDLWVISTDTYPPQVQGQTRYNVSASTTAQRLIGESWVIRYGDGSSANGIVYKDRVQIGNTFFNQQAVESAVNISNEISDDSFSSGLLGAASSAANTVRPDRQTTYLENIKSQLARPVFTANLKKGKPGNYNFGYINGSEYIGPIQYAAINPSSPLWEVSVSGYRVGSNDTKYVPRVWNAIADTGTTLLLVPNDIVSAYYAQVKGSTFSNDVGMMLVPCAATLPDFAFGLGNYRGVIPGSYINYGRMNKTYCYGGIQSSEDAPFAVLGDIALKAQFVVFDMGNKVVGFANKNTNV

SEQ ID NO: 22 - protein, 407 aa, Trichoderma reesei
MQTFGAFLVSFLAASGLAAALPTEGQKTASVEVQYNKNYVPHGPTALFKAKRKYGAPISDNLKSLVAARQAKQALAKRQTGSAPNHPSDSADSEYITSVSIGTPAQVLPLDFDTGSSDLWVFSSETPKSSATGHAIYTPSKSSTSKKVSGASWSISYGDGSSSSGDVYTDKVTIGGFSVNTQGVESATRVSTEFVQDTVISGLVGLAFDSGNQVRPHPQKTWFSNAASSLAEPLFTADLRHGQNGSYNFGYIDTSVAKGPVAYTPVDNSQGFWEFTASGYSVGGGKLNRNSIDGIADTGTTLLLLDDNVVDAYYANVQSAQYDNQQEGVVFDCDEDLPSFSFGVGSSTITIPGDLLNLTPLEEGSSTCFGGLQSSSGIGINIFGDVALKAALVVFDLGNERLGWAQK

SEQ ID NO: 23 - protein, 446 aa, Trichoderma reesei
MTLPVPLREHDLPFLKEKRKLPADDIPSGTYTLPIIHARRPKLASRAIEVQVENRSDVSYYAQLNIGTPPQTVYAQIDTGSFELWVNPNCSNVQSADQRFCRAIGFYDPSSSSTADVTSQSARLRYGIGSADVTYVHDTISLPGSGSGSKAMKAVQFGVADTSVDEFSGILGLGAGNGINTEYPNFVDELAAQGVTATKAFSLALGSKAEEEGVIIFGGVDTAKFHGELAHLPIVPADDSPDGVARYWVKMKSISLTPPPPSSSGSTDDNNNKPVAFPQTSMTVFLDSGSTLTLLPPALVRQIASALGSTQTDESGFFVVDCALASQDGTIDFEFDGVTIRVPYAEMIRQVSTLPPHCYLGMMGSTQFALLGDTFLRSAYAVFDLTSNVVHLAPYANCGTNVKSITSTSSLSNLVGTCNDPSKPSSSPSPSQTPSASPSSTATQKA

SEQ ID NO: 24 - protein, 259 aa, Trichoderma reesei
MAPASQVVSALMLPALALGAAIQPRGADIVGGTAASLGEFPYIVSLQNPNQGGHFCGGVLVNANTVVTAAHCSVVYPASQIRVRAGTLTWNSGGTLVGVSQIIVNPSYNDRTTDFDVAVWHLSSPIRESSTIGYATLPAQGSDPVAGSTVTTAGWGTTSENSNSIPSRLNKVSVPVVARSTCQADYRSQGLSVTNNMFCAGLTQGGKDSCSGDSGGPIVDANGVLQGVVSWGIGCAEAGFPGVYTRIGNFVNYINQNLA

SEQ ID NO: 25 - protein, 882 aa, Trichoderma reesei
MVRSALFVSLLATFSGVIARVSGHGSKIVPGAYIFEFEDSQDTADFYKKLNGEGSTRLKFDYKLFKGVSVQLKDLDNHEAKAQQMAQLPAVKNVWPVTLIDAPNPKVEWVAGSTAPTLESRAIKKPPIPNDSSDFPTHQMTQIDKLRAKGYTGKGVRVAVIDTGIDYTHPALGGCFGRGCLVSFGTDLVGDDYTGFNTPVPDDDPVDCAGHGSHVAGIIAAQENPYGFTGGAPDVTLGAYRVFGCDGQAGNDVLISAYNQAFEDGAQIITASIGGPSGWAEEPWAVAVTRIVEAGVPCTVSAGNEGDSGLFFASTAANGKKVIAVASVDNENIPSVLSVASYKIDSGAAQDFGYVSSSKAWDGVSKPLYAVSFDTTIPDDGCSPLPDSTPDLSDYIVLVRRGTCTFVQKAQNVAAKGAKYLLYYNNIPGALAVDVSAVPEIEAVGMVDDKTGATWIAALKDGKTVTLTLTDPIESEKQIQFSDNPTTGGALSGYTTWGPTWELDVKPQISSPGGNILSTYPVALGGYATLSGTSMACPLTAAAVALIGQARGTFDPALIDNLLATTANPQLFNDGEKFYDFLAPVPQQGGGLIQAYDAAFATTLLSPSSLSFNDTDHFIKKKQITLKNTSKQRVTYKLNHVPTNTFYTLAPGNGYPAPFPNDAVAAHANLKFNLQQVTLPAGRSITVDVFPTPPRDVDAKRLALWSGYITVNGTDGTSLSVPYQGLTGSLHKQKVLYPEDSWIADSTDESLAPVENGTVFTIPAPGNAGPDDKLPSLVVSPALGSRYVRVDLVLLSAPPHGTKLKTVKFLDTTSIGQPAGSPLLWISRGANPIAWTGELSDNKFAPPGTYKAVFHALRIFGNEKKKEDWDVSESPAFTIKYA

SEQ ID NO: 26 - protein, 541 aa, Trichoderma reesei
MRSVVALSMAAVAQASTFQIGTIHEKSAPVLSNVEANAIPDAYIIKFKDHVGEDDASKHHDWIQSIHTNVEQERLELRKRSNVFGADDVFDGLKHTFKIGDGFKGYAGHFHESVIEQVRNHPDVEYIERDSIVHTMLPLESKDSIIVEDSCNGETEKQAPWGLARISHRETLNFGSFNKYLYTADGGEGVDAYVIDTGTNIEHVDFEGRAKWGKTIPAGDEDEDGNGHGTHCSGTVAGKKYGVAKKAHVYAVKVLRSNGSGTMSDVVKGVEYAALSHIEQVKKAKKGKRKGFKGSVANMSLGGGKTQALDAAVNAAVRAGVHFAVAAGNDNADACNYSPAAATEPLTVGASALDDSRAYFSNYGKCTDIFAPGLSIQSTWIGSKYAVNTISGTSMASPHICGLLAYYLSLQPAGDSEFAVAPITPKKLKESVISVATKNALSDLPDSDTPNLLAWNGGGCSNFSQIVEAGSYTVKPKQNKQAKLPSTIEELEEAIEGDFEVVSGEIVKGAKSFGSKAEKFAKKIHDLVEEEIEEFISELSE

SEQ ID NO: 27 - protein, 391 aa, Trichoderma reesei
MRLSVLLSVLPLVLAAPAIEKRAEPAPLLVPTTKHGLVADKYIVKFKDGSSLQAVDEAISGLVSNADHVYQHVFRGFAATLDKETLEALRNHPEVDYIEQDAVVKINAYVSQTGAPWGLGRISHKARGSTTYVYDDSAGAGTCSYVIDTGVDATHPDFEGRATLLRSFVSGQNTDGNGHGTHVSGTIGSRTYGVAKKTQIYGVKVLDNSGSGSFSTVIAGMDYVASDSQTRNCPNGSVANMSLGGGYTASVNQAAARLIQAGVFLAVAAGNDGVDARNTSPASEPTVCTVGASTSSDARASFSNYGSVVDIFAPGQDILSTWPNRQTNTISGTSMATPHIVGLGAYLAGLEGFSDPQALCARIQSLANRNLLSGIPSGTINAIAFNGNPSG

SEQ ID NO: 28 - protein, 387 aa, Trichoderma reesei
MGLVTNPFAKNIIPNRYIVVYNNSFGEEAISAKQAQFAAKIAKRNLGKRGLFGNELSTAIHSFSMHTWRAMALDADDIMIKDIFDAEEVAYIEADTKVQHAALVAQTNAAPGLIRLSNKAVGGQNYIFDNSAGSNITAYVVDTGIRITHSEFEGRATFGANFVNDDTDENGHGSHVAGTIGGATFGVAKNVELVAVKVLDADGSGSNSGVLNGMQFVVNDVQAKKRSGKAVMNMSLGGSFSTAVNNAITALTNAGIVPVVAAGNENQDTANTSPGSAPQAITVGAIDATTDIRAGFSNFGTGVDIYAPGVDVLSVGIKSDIDTAVLSGTSMASPHVAGLAAYLMALEGVSNVDDVSNLIKNLAAKTGAAVKQNIAGTTSLIANNGNF

SEQ ID NO: 29 - protein, 409 aa, Trichoderma reesei
MASLRRLALYLGALLPAVLAAPAVNYKLPEAVPNKFIVTLKDGASVDTDSHLTWVKDLHRRSLGKRSTAGVEKTYNIDSWNAYAGEFDEETVKQIKANPDVASVEPDYIMWLSDIVEDKRALTTQTGAPWGLGTVSHRTPGSTSYIYDTSAGSGTFAYVVDSGINIAHQQFGGRASLGYNAAGGDHVDTLGHGTHVSGTIGGSTYGVAKQASLISVKVFQGNSASTSVILDGYNWAVNDIVSRNRASKSAINMSLGGPASSTWATAINAAFNKGVLTIVAAGNGDALGNPQPVSSTSPANVPNAITVAALDINWRTASFTNYGAGVDVFAPGVNILSSWIGSNTATNTISGTSMATPHVVGLALYLQALEGLSTPTAVTNRIKALATTGRVTGSLNGSPNTLIFNGNSA

SEQ ID NO: 30 - protein, 555 aa, Trichoderma reesei
MRACLLFLGITALATAIPALKPPHGSPDRAHTTQLAKVSIALQPECRELLEQALHHLSDPSSPRYGRYLGREEAKALLRPRREATAAVKRWLARAGVPAHDVLTDGQFIHVRTLAEKAQALLGFEYNSTLGSQTIAISTLPGKIRKHVMTVQYVPLWTEADWEECKTIITPSCLKRLYHVDSYRAKYESSSLFGIVGFSGQAAQHDELDKFLHDFAPYSTNANFSIESVNGGQSPQGMNEPASEANGDVQYAVAMGYHVPVRYYAVGGENHDIIPDLDLVDTTEEYLEPFLEFASHLLDLDDDELPRVVSISYGANEQLFPRSYAHQVCDMFGQLGARGVSIVVAAGDLGPGVSCQSNDGSARPKFIPSFPATCPYVTSVGSTRGIMPEVAASFSSGGFSDYFARPAWQDRAVGAYLGAHGEEWEGFYNPAGRGFPDVAAQGVNFRFRAHGNESLSSGTSLSSPVFAALIALLNDHRSKSGMPPMGFLNPWIYTVGSHAFTDIIEARSEGCPGQSVEYLASPYIPNAGWSAVPGWDPVTGWGTPLFDRMLNLSLV

SEQ ID NO: 31 - protein, 388 aa, Trichoderma reesei
MAWLKKLALVLLAIVPYATASPALSPRSREILSLEDLESEDKYVIGLKQGLSPTDLKKHLLRVSAVQYRNKNSTFEGGTGVKRTYAIGDYRAYTAVLDRDTVREIWNDTLEKPPWGLATLSNKKPHGFLYRYDKSAGEGTFAYVLDTGINSKHVDFEGRAYMGFSPPKTEPTDINGHGTHVAGIIGGKTFGVAKKTQLIGVKVFLDDEATTSTLMEGLEWAVNDITTKGRQGRSVINMSLGGPYSQALNDAIDHIADMGILPVAAAGNKGIPATFISPASADKAMTVGAINSDWQETNFSNFGPQVNILAPGEDVLSAYVSTNTATRVLSGTSMAAPHVAGLALYLMALEEFDSTQKLTDRILQLGMKNKVVNLMTDSPNLIIHNNVK

SEQ ID NO: 32 - protein, 256 aa, Trichoderma reesei
MFIAGVALSALLCADTVLAGVAQDRGLAARLARRAGRRSAPFRNDTSHATVQSNWGGAILEGSGFTAASATVNVPRGGGGSNAAGSAWVGIDGASCQTAILQTGFDWYGDGTYDAWYEWYPEFAADFSGIDIRQGDQIAMSVVATSLTGGSATLENLSTGQKVTQNFNRVTAGSLCETSAEFIIEDFEECNSNGSNCQPVPFASFSPAITFSSATATRSGRSVSLSGAEITEVIVNNQDLTRCSVSGSSTLTCSYV

SEQ ID NO: 33 - protein, 236 aa, Trichoderma reesei
MDAIRARSAARRSNRFQAGSSKNVNGTADVESTNWAGAAITTSGVTEVSGTFTVPRPSVPAGGSSREEYCGAAWVGIDGYSDADLIQTGVLWCVEDGEYLYEAWYEYLPAALVEYSGISVTAGSVVTVTATKTGTNSGVTTLTSGGKTVSHTFSRQNSPLPGTSAEWIVEDFTSGSSLVPFADFGSVTFTGATAVVNGATVTAGGDSPVIIDLEDSRGDILTSTTVSGSTVTVEYE

SEQ ID NO: 34 - protein, 612 aa, Trichoderma reesei
MAKLSTLRLASLLSLVSVQVSASVHLLESLEKLPHGWKAAETPSPSSQIVLQVALTQQNIDQLESRLAAVSTPTSSTYGKYLDVDEINSIFAPSDASSSAVESWLQSHGVTSYTKQGSSIWFQTNISTANAMLSTNFHTYSDLTGAKKVRTLKYSIPESLIGHVDLISPTTYFGTTKAMRKLKSSGVSPAADALAARQEPSSCKGTLVFEGETFNVFQPDCLRTEYSVDGYTPSVKSGSRIGFGSFLNESASFADQALFEKHFNIPSQNFSVVLINGGTDLPQPPSDANDGEANLDAQTILTIAHPLPITEFITAGSPPYFPDPVEPAGTPNENEPYLQYYEFLLSKSNAEIPQVITNSYGDEEQTVPRSYAVRVCNLIGLLGLRGISVLHSSGDEGVGASCVATNSTTPQFNPIFPATCPYVTSVGGTVSFNPEVAWAGSSGGFSYYFSRPWYQQEAVGTYLEKYVSAETKKYYGPYVDFSGRGFPDVAAHSVSPDYPVFQGGELTPSGGTSAASPVVAAIVALLNDARLREGKPTLGFLNPLIYLHASKGFTDITSGQSEGCNGNNTQTGSPLPGAGFIAGAHWNATKGWDPTTGFGVPNLKKLLALVRF

SEQ ID NO: 35 - protein, 477 aa, Trichoderma reesei
MRFVQYVSLAGLFAAATVSAGVVTVPFEKRNLNPDFAPSLLRRDGSVSLDAINNLTGGGYYAQFSVGTPPQKLSFLLDTGSSDTWVNSVTADLCTDEFTQQTVGEYCFRQFNPRRSSSYKASTEVFDITYLDGRRIRGNYFTDTVTINQANITGQKIGLALQSVRGTGILGLGFRENEAADTKYPTVIDNLVSQKVIPVPAFSLYLNDLQTSQGILLFGGVDTDKFHGGLATLPLQSLPPSIAETQDIVMYSVNLDGFSASDVDTPDVSAKAVLDSGSTITLLPDAVVQELFDEYDVLNIQGLPVPFIDCAKANIKDATFNFKFDGKTIKVPIDEMVLNNLAAASDEIMSDPSLSKFFKGWSGVCTFGMGSTKTFGIQSDEFVLLGDTFLRSAYVVYDLQNKQIGIAQATLNSTSSTIVEFKAGSKTIPGPASTGDDSDDSSDDSDEDSAGAALHPTFSIALAGTLFTAVSMMMSVL

SEQ ID NO: 36 - DNA, 1263 bp, Trichoderma reesei
atggcgtcactcatcaaaactgccgtggacattgccaacggccgccatgcgctgtccagatatgtcatctttgggctctggcttgcggatgcggtgctgtgcgggctgattatctggaaagtgccttatacggaaatcgactgggtcgcctacatggagcaagtcacccagttcgtccacggagagcgagactaccccaagatggagggcggcacagggcccctggtgtatcccgcggcccatgtgtacatctacacagggctctactacctgacgaacaagggcaccgacatcctgctggcgcagcagctctttgccgtgctctacatggctactctggcggtcgtcatgacatgctactccaaggccaaggtcccgccgtacatcttcccgcttctcatcctctccaaaagacttcacagcgtcttcgtcctgagatgcttcaacgactgcttcgccgccttcttcctctggctctgcatcttcttcttccagaggcgagagtggaccatcggagctctcgcatacagcatcggcctgggcgtcaaaatgtcgctgctactggttctccccgccgtggtcatcgtcctctacctcggccgcggcttcaagggcgccctgcggctgctctggctcatggtgcaggtccagctcctcctcgccatacccttcatcacgacaaattggcgcggctacctcggccgtgcattcgagctctcgaggcagttcaagtttgaatggacagtcaattggcgcatgctgggcgaggatctgttcctcagccggggcttctctatcacgctactggcatttcacgccatcttcctcctcgcctttatcctcggccggtggctgaagattagggaacggaccgtactcgggatgatcccctatgtcatccgattcagatcgccctttaccgagcaggaagagcgcgccatctccaaccgcgtcgtcacgcccggctatgtcatgtccaccatcttgtcggccaacgtggtgggactgctgtttgcccggtctctgcactaccagttctatgcatatctggcgtgggcgaccccctatctcctgtggacggcctgccccaatcttttggtggtggcccccctctgggcggcgcaagaatgggcctggaacgtcttccccagcacgcctcttagctcgagcgtcgtggtgagcgtgctggccgtgacggtggccatggcgtttgcaggttcaaatccgcagccacgtgaaacatcgaagccgaagcagcactaa

SEQ ID NO: 37 - protein, 420 aa, Trichoderma reesei
MASLIKTAVDIANGRHALSRYVIFGLWLADAVLCGLIIWKVPYTEIDWVAYMEQVTQFVHGERDYPKMEGGTGPLVYPAAHVYIYTGLYYLTNKGTDILLAQQLFAVLYMATLAVVMTCYSKAKVPPYIFPLLILSKRLHSVFVLRCFNDCFAAFFLWLCIFFFQRREWTIGALAYSIGLGVKMSLLLVLPAVVIVLYLGRGFKGALRLLWLMVQVQLLLAIPFITTNWRGYLGRAFELSRQFKFEWTVNWRMLGEDLFLSRGFSITLLAFHAIFLLAFILGRWLKIRERTVLGMIPYVIRFRSPFTEQEERAISNRVVTPGYVMSTILSANVVGLLFARSLHYQFYAYLAWATPYLLWTACPNLLVVAPLWAAQEWAWNVFPSTPLSSSVVVSVLAVTVAMAFAGSNPQPRETSKPKQH

SEQ ID NO: 38 - protein, 445 aa, Homo sapiens
MLKKQSAGLVLWGAILFVAWNALLLLFFWTRPAPGRPPSVSALDGDPASLTREVIRLAQDAEVELERQRGLLQQIGDALSSQRGRVPTAAPPAQPRVPVTPAPAVIPILVIACDRSTVRRCLDKLLHYRPSAELFPIIVSQDCGHEETAQAIASYGSAVTHIRQPDLSSIAVPPDHRKFQGYYKIARHYRWALGQVFRQFRFPAAVVVEDDLEVAPDFFEYFRATYPLLKADPSLWCVSAWNDNGKEQMVDASRPELLYRTDFFPGLGWLLLAELWAELEPKWPKAFWDDWMRRPEQRQGRACIRPEISRTMTFGRKGVSHGQFFDQHLKFIKLNQQFVHFTQLDLSYLQREAYDRDFLARVYGAPQLQVEKVRTNDRKELGEVRVQYTGRDSFKAFAKALGVMDDLKSGVPRAGYRGIVTFQFRGRRVHLAPPLTWEGYDPSWN

SEQ ID NO: 39 - protein, 447 aa, Homo sapiens
MRFRIYKRKVLILTLVVAACGFVLWSSNGRQRKNEALAPPLLDAEPARGAGGRGGDHPSVAVGIRRVSNVSAASLVPAVPQPEADNLTLRYRSLVYQLNFDQTLRNVDKAGTWAPRELVLVVQVHNRPEYLRLLLDSLRKAQGIDNVLVIFSHDFWSTEINQLIAGVNFCPVLQVFFPFSIQLYPNEFPGSDPRDCPRDLPKNAALKLGCINAEYPDSFGHYREAKFSQTKHHWWWKLHFVWERVKILRDYAGLILFLEEDHYLAPDFYHVFKKMWKLKQQECPECDVLSLGTYSASRSFYGMADKVDVKTWKSTEHNMGLALTRNAYQKLIECTDTFCTYDDYNWDWTLQYLTVSCLPKFWKVLVPQIPRIFHAGDCGMHHKKTCRPSTQSAQIESLLNNNKQYMFPETLTISEKFTVVAISPPRKNGGWGDIRDHELCKSYRRLQ

SEQ ID NO: 40 - protein, 85 aa, Trichoderma reesei
MASTNARYVRYLLIAFFTILVFYFVSNSKYEGVDLNKGTFTAPDSTKTTPKPPATGDAKDFPLALTPNDPGFNDLVGIAPGPRMN

SEQ ID NO: 41 - DNA, 255 bp, Trichoderma reesei
atggcgtcaacaaatgcgcgctatgtgcgctatctactaatcgccttcttcacaatcctcgtcttctactttgtctccaattcaaagtatgagggcgtcgatctcaacaagggcaccttcacagctccggattcgaccaagacgacaccaaagccgccagccactggcgatgccaaagactttcctctggccctgacgccgaacgatccaggcttcaacgacctcgtcggcatcgctcccggccctcgaatgaac

SEQ ID NO: 42 - protein, 58 aa, Homo sapiens
MRFRIYKRKVLILTLVVAACGFVLWSSNGRQRKNEALAPPLLDAEPARGAGGRGGDHP

SEQ ID NO: 43 - protein, 51 aa, Trichoderma reesei
MASTNARYVRYLLIAFFTILVFYFVSNSKYEGVDLNKGTFTAPDSTKTTPK

SEQ ID NO: 44 - protein, 52 aa, Trichoderma reesei
MAIARPVRALGGLAAILWCFFLYQLLRPSSSYNSPGDRYINFERDPNLDPTG

SEQ ID NO: 45 - protein, 33 aa, Trichoderma reesei
MLNPRRALIAAAFILTVFFLISRSHNSESASTS

SEQ ID NO: 46 - protein, 84 aa, Trichoderma reesei
MMPRHHSSGFSNGYPRADTFEISPHRFQPRATLPPHRKRKRTAIRVGIAVVVILVLVLWFGQPRSVASLISLGILSGYDDLKLE

SEQ ID NO: 47 - protein, 55 aa, Trichoderma reesei
MLLPKGGLDWRSARAQIPPTRALWNAVTRTRFILLVGITGLILLLWRGVSTSASE

SEQ ID NO: 48 - DNA, 20 bp, Artificial
  Primer
ccgcgttgaacggcttccca

SEQ ID NO: 49 - DNA, 24 bp, Artificial
  Primer
taacttgtacgctctcagttcgag

SEQ ID NO: 50 - DNA, 20 bp, Artificial
  Primer
gcgacggcgacccattagca

SEQ ID NO: 51 - DNA, 19 bp, Artificial
  Primer
catcctcaaggcctcagac

SEQ ID NO: 52 - DNA, 20 bp, Artificial
  Primer
tgcgctctcaccagcatcgc

SEQ ID NO: 53 - DNA, 20 bp, Artificial
  Primer
gtcctgggcgagttccgcac

SEQ ID NO: 54 - DNA, 63 bp, Artificial
  Primer
agatttcagtctctcaccactcacctgagttgcctctctcggtctgaaggacgtggaatgatg

SEQ ID NO: 55 - DNA, 66 bp, Artificial
  Primer
gcagggtgatgagctggatcaccttgacggtgttgcccatgttgagagaagttgttggattgatca

SEQ ID NO: 56 - DNA, 63 bp, Artificial; identical to SEQ ID NO: 54
  Primer

SEQ ID NO: 57 - DNA, 66 bp, Artificial
  Primer
cagagccgctatcgccgaggaggttgcccttcttgcccatgttgagagaagttgttggattgatca

SEQ ID NO: 58 - DNA, 63 bp, Artificial; identical to SEQ ID NO: 54
  Primer

SEQ ID NO: 59 - DNA, 66 bp, Artificial
  Primer
tcttgaggatgagctggacgagggtcttgaaaaagcccatgttgagagaagttgttggattgatca

SEQ ID NO: 60 - DNA, 21 bp, Artificial
  Primer
agctccgtggcgaaagcctga

SEQ ID NO: 61 - DNA, 66 bp, Artificial
  Primer
cagccgcagcctcagcctctctcagcctcatcagccgcggccgccaactttgcgtcccttgtgacg

SEQ ID NO: 62 - DNA, 76 bp, Artificial
  Primer
gcaacgagagcagagcagcagtagtcgatgctaggcggccgcgggcagtatgccggatggctggcttatacaggca

SEQ ID NO: 63 - DNA, 76 bp, Artificial
  Primer
tgcctgtataagccagccatccggcatactgcccgcggccgcctagcatcgactactgctgctctgctctcgttgc

SEQ ID NO: 64 - DNA, 20 bp, Artificial
  Primer
tgcgtcgccgtctcgctcct

SEQ ID NO: 65 - DNA, 20 bp, Artificial
  Primer
ttaggcgacctctttttcca

SEQ ID NO: 66 - DNA, 20 bp, Artificial
  Primer
cgaggaagtctcgtgaggat

SEQ ID NO: 67 - DNA, 19 bp, Artificial
  Primer
cagctaaaccgacgggcca

SEQ ID NO: 68 - DNA, 20 bp, Artificial
  Primer
gaccgtatatttgaaaaggg

SEQ ID NO: 69 - DNA, 20 bp, Artificial
  Primer
gatgttgcgcctgggttgac

SEQ ID NO: 70 - DNA, 23 bp, Artificial
  Primer
taacttgtacgctctcagttcga

SEQ ID NO: 71 - DNA, 20 bp, Artificial
  Primer
ccatgagcttgaacaggtaa

SEQ ID NO: 72 - DNA, 20 bp, Artificial
  Primer
gattgtcatggtgtacgtga

SEQ ID NO: 73 - DNA, 20 bp, Artificial
  Primer
caagatggagggcggcacag

SEQ ID NO: 74 - DNA, 22 bp, Artificial
  Primer
gccagtagcgtgatagagaagc

SEQ ID NO: 75 - DNA, 20 bp, Artificial
  Primer
gcgtcactcatcaaaactgc

SEQ ID NO: 76 - DNA, 19 bp, Artificial
  Primer
cttcggcttcgatgtttca

SEQ ID NO: 77 - DNA, 20 bp, Artificial
  Primer
tgcgtcgccgtctcgctcct

SEQ ID NO: 78 - DNA, 20 bp, Artificial
  Primer
tgacgtaccagttgggatga

SEQ ID NO: 79 - DNA, 20 bp, Artificial
  Primer
gatgttgcgcctgggttgac

SEQ ID NO: 80 - DNA, 20 bp, Artificial
  Primer
tgacgtaccagttgggatga

SEQ ID NO: 81 - DNA, 20 bp, Artificial
  Primer
tgcgtcgccgtctcgctcct

SEQ ID NO: 82 - DNA, 20 bp, Artificial
  Primer
gattgtcatggtgtacgtga

SEQ ID NO: 83 - DNA, 20 bp, Artificial
  Primer
caagatggagggcggcacag

SEQ ID NO: 84 - DNA, 22 bp, Artificial
  Primer
gccagtagcgtgatagagaagc

SEQ ID NO: 85 - protein, 488 aa, Trichoderma reesei
MRASPLAVAGVALASAAQAQVVQFDIEKRHAPRLSRRDGTIDGTLSNQRVQGGYFINVQVGSPGQNITLQLDTGSSDVWVPSSTAAICTQVSERNPGCQFGSFNPDDSDTFDEVGQGLFDITYVDGSSSKGDYFQDNFQINGVTVKNLTMGLGLSSSIPNGLIGVGYMNDEASVSTTRSTYPNLPIVLQQQKLINSVAFSLWLNDLDASTGSILFGGIDTEKYHGDLTSIDIISPNGGKTFTEFAVNLYSVQATSPSGTDTLSTSEDTLIAVLDSGTTLTYLPQDMAEEAWNEVGAEYSNELGLAVVPCSVGNTNGFFSFTFAGTDGPTINVTLSELVLDLFSGGPAPRFSSGPNKGQSICEFGIQNGTGSPFLLGDTFLRSAFVVYDLVNNQIAIAPTNFNSTRTNVVAFASSGAPIPSATAAPNQSRTGHSSSTHSGLSAASGFHDGDDENAGSLTSVFSGPGMAVVGMTICYTLLGSAIFGIGWL

SEQ ID NO: 86 - protein, 761 aa, Trichoderma reesei
MRSTLYGLAALPLAAQALEFIDDTVAQQNGIMRYTLTTTKGATSKHLHRRQDSADLMSQQTGYFYSIQLEIGTPPQAVSVNFDTGSSELWVNPVCSKATDPAFCKTFGQYNHSTTFVDAKAPGGIKYGTGFVDFNYGYDYVQLGSLRINQQVFGVATDSEFASVGILGAGPDLSGWTSPYPFVIDNLVKQGFIKSRAFSLDIRGLDSDRGSVTYGGIDIKKFSGPLAKKPIIPAAQSPDGYTRYWVHMDGMSITKEDGSKFEIFDKPNGQPVLLDSGYTVSTLPGPLMDKILEAFPSARLESTSGDYIVDCDIIDTPGRVNFKFGNVVVDVEYKDFIWQQPDLGICKLGVSQDDNFPVLGDTFLRAAYVVFDWDNQEVHIAANEDCGDELIPIGSGPDAIPASAIGKCSPSVKTDTTTSVAETTATSAAASTSELAATTSEAATTSSEAATTSAAAETTSVPLNTAPATTGLLPTTSHRFSNGTAPYPIPSLSSVAAAAGSSTVPSESSTGAAAAGTTSAATGSGSGSGSGDATTASATYTSTFTTTNVYTVTSCPPSVTNCPVGHVTTEVVVAYTTWCPVENGPHPTAPPKPAAPEITATFTLPNTYTCSQGKNTCSNPKTAPNVIVVTPIVTQTAPVVIPGIAAPTPTPSVAASSPASPSVVPSPTAPVATSPAQSAYYPPPPPPEHAVSTPVANPPAVTPAPAPFPSGGLTTVIAPGSTGVPSQPAQSGLPPVPAGAAGFRAPAAVALLAGAVAAALL

SEQ ID NO: 87 - protein, 526 aa, Trichoderma reesei
MRPNSVLLAPLALYASGALAFYPYTPPWLKELEEHNAGEAKRSADNGLTFDIKRRASRRAPASQEEKAAWQAALLSHKYSESVTPSPSPDTTLSKRDNQFSILKAVDPDAPNTAGLAQDGTDYSYFVQASLGSKKTKLYMLLDTGAGSSWVMGTDCVSEACSLHDSFGPEDSDTLKTSTKDFSIAYGSGAVSGSLVNDTIEVAGMSLTYQFGLAHNTSSDFVHFAFDGILGMSMNSGANENFLSALEGAGLLDKSIFSVALARASDGHNDGEVTFGATNPSRYTGDITYTPIPSGTDWSIPLDDMSYNGKKGNVGGINAYIDTGTSYMFGPSKNVKALHAVIDGAKSSDGITWTVPCDTTTPLVVTFSGVDFAISPKDWISPKDSSGKCTSNVYGYEVVSGSWLFGDTFLKNVYAVFDKEQMRIGKTSPRATSPSSPAPTRTPSPATTSPSSASTPGSTPTTSSTRTARPSTSAPSGTSSTGAPSPSASANRDVLRAKRINMLKSISSFWHDPCCCLFLHVSISST

SEQ ID NO: 88 - DNA, 2559 bp, Leishmania mexicana
atggggaaaaataaggcaaattcagtggccgactccggctctgcggcaaccgcacctcgtgaagctcctgcccaagccaaagatgccgccccacaagcccagaccgcatctccaccgcctaagaagactttgttgcccaaaacgctaacagatgagacggaatttgtcggcatctttccgttccctttctggccagtacggttcgtcgttacggtggtggcactcttcggcttaggcgccagctgcctccaagccttcacggttcgcatgacctcggttaagatttacggatacctgatccacgagttcgacccgtggttcaactaccgcgctgccgagtacatgtccacgcacggctggtccgccttcttcagctggttcgactacatgagctggtacccgctgggccgccccgtcggctccaccacgtacccgggcctgcagttcactgccgtcgccattcaccgcgcactggcggctgccggcatcccgatgtctctcaacgacgtgtgtgtgctgatcccggcgtggtttggcgccatcgctaccgctcttctggctctttgcacgtacgaagccagtgggtcgacggtggcggccgccgctgccgccctctccttctccatcatcccagcccacctgatgcggtccatggcgggtgagttcgacaacgagtgcatcgccgtcgccgccatgctgctcaccttctactgctgggtgcgctcgctgcgcacgcggtcctcgtggcccatcggcgtcctcaccggtgtcgcctacggctacatggtggcggcgtggggcggctacattttcgtgctcaacatggttgccatgcatgccggcatatcatcgatggtggactgggcccgcaacacgtacaacccgtcgctgctgcgtgcatacacgctgttctacgttgtcggcaccgccatcgccgtgtgcgtgccgccagtggggatgtcgcccttcaagtcgctggagcagctgggtgcgctgctggtgcttgtcttcctgtgcgggctgcaggtgtgcgaggtgctgcgggcacgcgccggtgtcgaggttcgctctcgcgcgaacttcaagatccgcgcgcgcgtcttcagcgcgatggctggcggggctgcgcttgcaatcgcgctgctggcaccgagggggtacttcgggcccctttcggctcgtgtgcgtgcgctgttcgtggagcacacgcgcactggcaatccgctggtcgactcggtcgccgaacatcaacccgccagccctgaggcaatgtggtcgtttcttcacgtgtgcggcgtgacatggggcttgggcttcattgtgcttgctgtctcaacgttcgtgaactactccccgtcgaaggtcttctgggtactgaactctggtgccgtgtactacttcagcacccgcatggctcggctgctgcttctctccggtcccgctgcgtgtctgtccactggcattttcgtgggggcaattctggaagcagcggtgcagctcagcttttgggacagtgatgcgacaaaggccaagccccagaagcagacccaacgccaccagaggggggctcgtaaggacaacaagcgaaatgacgctgagagcggaatgaccgcgctctcactttgcgacatcgtgtccggtagctctctggcttggggccatcgtatggtgctgtgcatcgctatgtgggctctcgtgacgacaaccgtggtgaccttcatcagttccggtttcgcgtcccactcactaaaatttgcggagcagtcgtcaaatccgatgattgttttcgcggcctccgtgccaaaccgtgcaacaggcaagcctatgatgatattggtggatgactacctgcacagctatctctggctgcgcgataacacacccaggagtgcgcgcattttggcctggtgggactacggctaccagatcacaggcatcggcaaccgcacctcgctggccgatggcaacacctggaaccacgagcacatcgccaccatcggcaagatgttgacgtcgcccgtggcggaggcgcactcgctggtgcgccacatggccgactacgtcctcatctgggctgggcagagcggagacttgatgaagtcaccgcacatggcgcgcatcggcaacagtgtgtaccacgacatctgccccaacgacccgctgtgccagcaattcggcttttacagaaatgattaccatcgtccaacaccgatgatgcgggcgtcgctgctgtacaacctgcacgaggccgggaaaacagcggccgtgaaggtggacccatccctctttcaggaggtgtactcgtccaagtacggcctggtgcgcatcttcaaggtcatgaacgtgagcgcggagagcaagaagtgggttgctgacccggcaaaccgcgtgtgccgcccgcctgggtcgtggatctgccccgggcagtacccgccggcgaaggagatccaggagatgctggcacaccgggtctccttcgatcaggtggacaaggacaagaagcgcaaggcgacgtaccacgaggagtacatgcgccggatgcgtgaaaacgagatctga

SEQ ID NO: 89 - protein, 852 aa, Leishmania mexicana
MGKNKANSVADSGSAATAPREAPAQAKDAAPQAQTASPPPKKTLLPKTLTDETEFVGIFPFPFWPVRFVVTVVALFGLGASCLQAFTVRMTSVKIYGYLIHEFDPWFNYRAAEYMSTHGWSAFFSWFDYMSWYPLGRPVGSTTYPGLQFTAVAIHRALAAAGIPMSLNDVCVLIPAWFGAIATALLALCTYEASGSTVAAAAAALSFSIIPAHLMRSMAGEFDNECIAVAAMLLTFYCWVRSLRTRSSWPIGVLTGVAYGYMVAAWGGYIFVLNMVAMHAGISSMVDWARNTYNPSLLRAYTLFYVVGTAIAVCVPPVGMSPFKSLEQLGALLVLVFLCGLQVCEVLRARAGVEVRSRANFKIRARVFSAMAGGAALAIALLAPRGYFGPLSARVRALFVEHTRTGNPLVDSVAEHQPASPEAMWSFLHVCGVTWGLGFIVLAVSTFVNYSPSKVFWVLNSGAVYYFSTRMARLLLLSGPAACLSTGIFVGAILEAAVQLSFWDSDATKAKPQKQTQRHQRGARKDNKRNDAESGMTALSLCDIVSGSSLAWGHRMVLCIAMWALVTTTVVTFISSGFASHSLKFAEQSSNPMIVFAASVPNRATGKPMMILVDDYLHSYLWLRDNTPRSARILAWWDYGYQITGIGNRTSLADGNTWNHEHIATIGKMLTSPVAEAHSLVRHMADYVLIWAGQSGDLMKSPHMARIGNSVYHDICPNDPLCQQFGFYRNDYHRPTPMMRASLLYNLHEAGKTAAVKVDPSLFQEVYSSKYGLVRIFKVMNVSAESKKWVADPANRVCRPPGSWICPGQYPPAKEIQEMLAHRVSFDQVDKDKKRKATYHEEYMRRMRENEI

SEQ ID NO: 90 - DNA, 2565 bp, Leishmania braziliensis
atgggtaagaagaaagcaattccgtcgggcagcgtcggccctgcgacaaccacctcccgtgaagctccaggcaaagacgaaggtgcctcccaacccgccaagactgcagctctgccggtgaagccctttgtgttgcccaacacgctgacagacgaggaggagtttgttggcatctttccctgccctttctggccagtgcgatttgtcatcacagtgatggcactcgtcctcttgggtgccagctgtatccgcgccttcacgattcgcatgctatccgttcagctttatggctacatcatccacgagttcgacccgtggttcaactaccgcgccgccgagtacatgtccgcgcacggctggtccgccttcttcagctggttcgactacatgagctggtacccgctgggccgccccgttggcaccaccacgtacccgggcctgcagctcaccgccgttgccatccaccgcgcattggcggctgccggggtgccgatgtctctcaacaacgtgtgcgtgctgatccccgcgtggtatggtgccatcgctactgctatcctggccctttgcgcttacgaggtcagtaggtcaatggtagcggcggctgttgctgcactctcattctccatcattccagcacacctgatgcggtccatggcgggcgagttcgacaacgagtgcatcgccgttgcagccatgctcctcaccttctacttgtgggtacgctcgctgcgcacgcggtgctcgtggcccatcggcatcctcaccggtatcgcctacggctacatggtggcggcgtggggcggatacatttttgtgctcaacatggttgccatgcacgccggcatatcatcgatggtcgactgggctcgcaacacgtacaacccgtcgctgctgcgcgcatacgcgctgttctacgttgtcggcaccgccatcgccacgcgcgtgccgcctgtggggatgtcgcccttcaggtcgctggagcagctgggtgcgctggcggtgctcctcttcctgtgcgggctgcaggcctgcgaggtgtttcgcgcacgggccgacgtcgaggttcgctcccgcgcgaacttcaagatccgcatgcgtgccttcagcgtgatggctggcgtgggtgcgcttgcaatcgcggtgctgtcgccgaccgggtactttggccccctcacggctcgtgtgcgtgcgctgttcatggagcacacgcgcactggcaatccgctggtcgactcggtcgctgagcaccaccccgccagtcctgaggcgatgtggacatttcttcacgtgtgcggcgtgacttggggtttgggctccattgttcttcttgtgtcgttgctggtggactactcctcggcaaagctcttttggctgatgaactctggtgccgtgtactatttcagcacccgcatgtcacgactgctgcttctcacgggccccgctgcgtgtctgtccactggctgtttcgtggggacattactggaagcggcgatacagttcaccttctggtccagcgatgcaacaaaggccaaaaaacagcaagagacacaacttcaccaaaagggcgcgcgcaagcatagcgaccggagtaactctaagaatgcactgactgtgcgtacattgggcgacgtcttgaggagtacctctctggcatggggtcatcgcatggtgctctgcttcgctatgtgggctcttgttattacagtcgcggtgtgcctcttgggttccgatttcacttcccatgcaacgatgtttgcaaggcagacgtcgaacccgctgattgtctttgcaaccgtgctgcgagaccgcgctaccggcaagccaacacaggtattggtggatgactacctgcgcagctatctctggctgcgcgacaacacgcccagaaatgcgcgcgtgctgtcctggtgggactacggctaccagatcacaggtatcggcaaccgcacctcgctggccgatggcaacacctggaaccacgagcacatcgccaccatcggcaagatgctgacgtcgcccgtggcggaggcgcactcactggtgcgccacatggcggactacgtcctcatctgggctgggcagggcggagacttgatgaagtcgccgcacatggcgcgcattggcaacagcgtgtaccacgacatctgccccaacgacccgctttgccagcatttcggcttttacaagaacgatcgcaatcgcccaaaaccgatgatgcgcgcgtcgctgctgtacaacctgcacgaggccggacgaagcgcgggtgtgaaggtggacccgtccctctttcaggaagtgtactcatccaagtacggcctggtgcgcatcttcaaggtcatgaacgtgagcgcggagagcaagaagtgggtggctgacccggcaaaccgcgtgtgccacccgcctgggtcgtggatctgccccgggcagtacccgccggcgaaggagatccaggagatgctggcgcaccgcgtcccctttgaccatgtgaacagcttcagtcggaaaaaggccgggtcttatcatgaagaatacatgcgccggatgcgtgaagagcaggaccgatga

SEQ ID NO: 91 - protein, 854 aa, Leishmania braziliensis
MGKKKAIPSGSVGPATTTSREAPGKDEGASQPAKTAALPVKPFVLPNTLTDEEEFVGIFPCPFWPVRFVITVMALVLLGASCIRAFTIRMLSVQLYGYIIHEFDPWFNYRAAEYMSAHGWSAFFSWFDYMSWYPLGRPVGTTTYPGLQLTAVAIHRALAAAGVPMSLNNVCVLIPAWYGAIATAILALCAYEVSRSMVAAAVAALSFSIIPAHLMRSMAGEFDNECIAVAAMLLTFYLWVRSLRTRCSWPIGILTGIAYGYMVAAWGGYIFVLNMVAMHAGISSMVDWARNTYNPSLLRAYALFYVVGTAIATRVPPVGMSPFRSLEQLGALAVLLFLCGLQACEVFRARADVEVRSRANFKIRMRAFSVMAGVGALAIAVLSPTGYFGPLTARVRALFMEHTRTGNPLVDSVAEHHPASPEAMWTFLHVCGVTWGLGSIVLLVSLLVDYSSAKLFWLMNSGAVYYFSTRMSRLLLLTGPAACLSTGCFVGTLLEAAIQFTFWSSDATKAKKQQETQLHQKGARKHSDRSNSKNALTVRTLGDVLRSTSLAWGHRMVLCFAMWALVITVAVCLLGSDFTSHATMFARQTSNPLIVFATVLRDRATGKPTQVLVDDYLRSYLWLRDNTPRNARVLSWWDYGYQITGIGNRTSLADGNTWNHEHIATIGKMLTSPVAEAHSLVRHMADYVLIWAGQGGDLMKSPHMARIGNSVYHDICPNDPLCQHFGFYKNDRNRPKPMMRASLLYNLHEAGRSAGVKVDPSLFQEVYSSKYGLVRIFKVMNVSAESKKWVADPANRVCHPPGSWICPGQYPPAKEIQEMLAHRVPFDHVNSFSRKKAGSYHEEYMRRMREEQDR

(19) United States
(12) Patent Application Publication
(10) Pub. No.: US 2019/0276867 A1
(43) Pub. Date: Sep. 12, 2019

(54) Production of Glycoproteins Having Increased N-Glycosylation Site Occupancy

(71) Applicant: Glykos Finland Oy, Helsinki (FI)

(72) Inventors: Jari Natunen, Vantaa (FI); Christopher Landowski, Helsinki (FI); Markku Saloheimo, Helsinki (FI); Christian Ostermeier, Basel (CH); Benjamin Patrick Sommer, Basel (CH); Ramon Wahl, Basel (CH)

(73) Assignee: Glykos Finland Oy, Helsinki (FI)

(21) Appl. No.: 16/364,698

(22) Filed: Mar. 26, 2019

Related U.S. Application Data
(63) Continuation of application No. 14/903,610, filed on Jan. 8, 2016, now abandoned.

(30) Foreign Application Priority Data
Jul. 10, 2013 (EP) 13175997.9

(51) Int. Cl. C12P 21/00; C12N 9/10; C12N 15/80; C07K 16/00

(52) U.S. Cl. CPC C12P 21/005; C12N 9/1051; C12Y 204/01; C12N 9/1081; C07K 16/00; C12N 15/80

(57) ABSTRACT

The present disclosure relates to compositions and methods useful for the production of heterologous proteins with increased N-glycosylation site occupancy in filamentous fungal cells, such as Trichoderma cells. More specifically, the invention provides a filamentous fungal cell comprising i. one or more mutation that reduces or eliminates one or more endogenous protease activity compared to a parental filamentous fungal cell which does not have said mutation(s), ii. a polynucleotide encoding a heterologous catalytic subunit of oligosaccharyl transferase, and iii. a polynucleotide encoding a heterologous glycoprotein, wherein said catalytic subunit of oligosaccharyl transferase is selected from Leishmania oligosaccharyl transferase catalytic subunits.

### FIELD OF THE INVENTION

The present disclosure relates to compositions and methods useful for the production of heterologous proteins, e.g recombinant antibodies, in filamentous fungal cells.

### BACKGROUND

Posttranslational modification of eukaryotic proteins, particularly therapeutic proteins such as immunoglobulins, is often necessary for proper protein folding and function. Because standard prokaryotic expression systems lack the proper machinery necessary for such modifications, alternative expression systems have to be used in production of these therapeutic proteins. Even where eukaryotic proteins do not have posttranslational modifications, prokaryotic expression systems often lack necessary chaperone proteins required for proper folding. Yeast and fungi are attractive options for expressing proteins as they can be easily grown at a large scale in simple media, which allows low production costs, and yeast and fungi have posttranslational machinery and chaperones that perform similar functions as found in mammalian cells. Moreover, tools are available to manipulate the relatively simple genetic makeup of yeast and fungal cells as well as more complex eukaryotic cells such as mammalian or insect cells (De Pourcq et al., Appl Microbiol Biotechnol, 87(5):1617-31).

However, posttranslational modifications occurring in yeast and fungi may still be a concern for the production of recombinant therapeutic protein. In particular, insufficient N-glycosylation is one of the biggest hurdles to overcome in the production of biopharmaceuticals for human applications in fungi.

N-glycosylation, which refers to the attachment of sugar molecule to a nitrogen atom of an asparagine side chain, has been shown to modulate the pharmacokinetics and pharmacodynamics of therapeutic proteins.

When recombinant proteins are expressed in filamentous fungal cells such as Trichoderma fungus cells, the proportion of N-glycosylation sites that are indeed glycosylated is generally lower than for the same protein expressed in a mammalian system, such as CHO cells.

WO2011/106389, entitled “Methods for increasing N-glycosylation site occupancy on therapeutic glycoproteins produced in Pichia pastoris”, describes Pichia pastoris cells that overexpress heterologous single-subunit oligotransferase, and are able to produce glycoproteins with improved N-glycosylation.

Similarly, Choi et al. describe improved N-glycosylation of recombinant proteins by heterologous expression of heterologous single-subunit oligotransferase (Choi et al., Appl Microbiol Biotechnol, 95(3): 671-82).

The same authors have also described, in WO2013062939, methods for increasing N-glycan occupancy and reducing production of hybrid N-glycans in Pichia pastoris strains lacking alpha-1,3 mannosyltransferase activity (Alg3p disruption).

Reports of fungal cell expression systems expressing human-like fucosylated N-glycans are lacking. Indeed, due to the industry's focus on mammalian cell culture technology for such a long time, the fungal cell expression systems such as Trichoderma are not as well established for therapeutic protein production as mammalian cell culture and therefore suffer from drawbacks when expressing mammalian proteins. In particular, a need remains in the art for improved filamentous fungal cells, such as Trichoderma fungus cells, that can stably produce heterologous proteins with increased N-glycosylation site occupancy, preferably at high levels of expression.

### SUMMARY

The present invention relates to improved methods for producing glycoproteins with increased N-glycosylation site occupancy in filamentous fungal expression systems, and more specifically, glycoproteins, such as antibodies or related immunoglobulins or fusion proteins.

The present invention is based in part on the surprising discovery that filamentous fungal cells, such as Trichoderma cells, can be genetically modified to express oligosaccharyl transferase activity, without adversely affecting yield of produced glycoproteins.

Accordingly, in a first aspect, the invention relates to a filamentous fungal cell comprising
i. one or more mutation that reduces or eliminates one or more endogenous protease activity compared to a parental filamentous fungal cell which does not have said mutation(s),
ii. a polynucleotide encoding a heterologous catalytic subunit of oligosaccharyl transferase, and
iii. a polynucleotide encoding a heterologous glycoprotein,

wherein said catalytic subunit of oligosaccharyl transferase is selected from Leishmania oligosaccharyl transferase catalytic subunits.

In one embodiment, said filamentous fungal cell has at least a two-fold reduction, preferably at least a three-fold reduction, even more preferably at least a four-fold reduction, at least a five-fold reduction, in total protease activity compared to a parental filamentous fungal cell which does not have the protease-deficient mutations(s).

In one embodiment of the invention, said filamentous fungal cell is a Trichoderma, Neurospora, Myceliophtora, Chrysosporium, Aspergillus, or Fusarium cell.

In one embodiment of the invention, the polynucleotide encoding the heterologous catalytic subunit of oliogaccharyl transferase comprises a nucleic acid sequence selected from the group consisting of SEQ ID NO: 2, SEQ ID NO: 9, SEQ ID NO: 88 and SEQ ID NO: 90 or a polynucleotide encoding a functional variant polypeptide having at least 50%, at least 60%, at least 70% identity, at least 80% identity, at least 90% identity, or at least 95% identity with SEQ ID NO: 1, SEQ ID NO: 8, SEQ ID NO: 89 or SEQ ID NO: 91, said functional variant polypeptide having oligosaccharyltransferase activity.

In another embodiment, said polynucleotide encoding the heterologous catalytic subunit of oligosaccharyl transferase is under the control of a promoter for constitutive expression of said oligosaccharyl transferase in said cell.

In one embodiment of the invention, the N-glycosylation site occupancy of the heterologous glycoprotein expressed in filamentous fungal cell is at least 80%, at least 90%, at least 95%, at least 99%, or 100%.

In a specific embodiment, the N-glycosylation site occupancy of the heterologous glycoprotein is at least 95% and Man3, Man5, G0, G1 and/or G2 glycoforms represent at least 50% of total neutral N-glycans of the heterologous glycoprotein.

In one embodiment of the invention, the filamentous fungal cell is a Trichoderma cell, for example, Trichoderma reesei, and said cell comprises mutations that reduce or eliminate the activity of
the three endogenous proteases pep1, tsp1, and slp1;
the three endogenous proteases gap1, slp1, and pep1;
the three endogenous proteases selected from the group consisting of pep1, pep2, pep3, pep4, pep5, pep8, pep9, pep11, pep12, tsp1, slp1, slp2, slp3, slp7, gap1 and gap2;
three to six proteases selected from the group consisting of pep1, pep2, pep3, pep4, pep5, tsp1, slp1, slp2, slp3, gap1 and gap2;
seven to ten proteases selected from the group consisting of pep1, pep2, pep3, pep4, pep5, pep7, pep8, pep9, tsp1, slp1, slp2, slp3, slp5, slp6, slp7, slp8, tpp1, gap1 and gap2.

In one embodiment, the fungal cell further comprises a mutation in the gene encoding ALG3 that reduces or eliminates the corresponding ALG3 expression compared to the level of expression of ALG3 gene in a parental cell which does not have such mutation.

In one embodiment, the fungal cell further comprises a polynucleotide encoding an N-acetylglucosaminyltransferase I catalytic domain and an N-acetylglucosaminyltransferase II catalytic domain.

In one embodiment, the fungal cell further comprises one or more polynucleotides encoding a polypeptide selected from the group consisting of:
i. α1,2 mannosidase;
ii. N-acetylglucosaminyltransferase I catalytic domain;
iii. α-mannosidase II;
iv. N-acetylglucosaminyltransferase II catalytic domain;
v. β1,4 galactosyltransferase; and,
vi. fucosyltransferase.

In one embodiment of the invention, the heterologous glycoprotein is a mammalian glycoprotein.

In a specific embodiment, said mammalian glycoprotein is selected from the group consisting of an antibody, an immunoglobulin or a protein fusion comprising Fc fragment of an immunoglobulin.

In another specific embodiment, said mammalian glycoprotein is a therapeutic antibody.

In another aspect, the invention also relates to a method of increasing N-glycosylation site occupancy of heterologous glycoprotein produced in a filamentous fungal host cell, comprising:

a) providing a filamentous fungal host cell, for example a Trichoderma cell, having a Leishmania STT3D gene encoding a catalytic subunit of oligosaccharyl transferase, or a functional variant thereof, and a polynucleotide encoding a heterologous glycoprotein,

b) culturing the host cell under appropriate conditions for expression of the STT3D gene or its functional variant, or said functional variant, and the production of the heterologous glycoprotein; wherein the expressed heterologous glycoproteins exhibit increased N-glycosylation site occupancy compared to the heterologous glycoproteins expressed in a corresponding parental filamentous fungal cell which does not express said oligosaccharyl transferase catalytic subunit.

The invention also relates to a method of producing a heterologous glycoprotein composition, with increased N-glycosylation site occupancy, comprising:

a) providing a filamentous fungal cell, for example a Trichoderma cell, having a Leishmania STT3D gene encoding a catalytic subunit of oligosaccharyl transferase, or a functional variant thereof, and a polynucleotide encoding a heterologous glycoprotein,

b) culturing the cell under appropriate conditions for expression of the STT3D gene or its functional variant, and the production of the heterologous glycoprotein composition; and,

c) recovering and, optionally, purifying the heterologous glycoprotein composition.

In certain embodiments of the method of the invention, said Leishmania STT3D gene encoding a catalytic subunit of oligosaccharyl transferase comprises a nucleic acid sequence selected from the group consisting of SEQ ID NO: 2, SEQ ID NO: 9, SEQ ID NO: 88 and SEQ ID NO: 90, or a polynucleotide encoding a functional variant polypeptide having at least 50%, at least 60%, at least 70% identity, at least 80% identity, at least 90% identity, or at least 95% identity with SEQ ID NO: 1, SEQ ID NO: 8, SEQ ID NO: 89 or SEQ ID NO: 91, said functional variant polypeptide having oligosaccharyltransferase activity.

In one embodiment, said polynucleotide encoding said heterologous glycoprotein further comprises a polynucleotide encoding CBH1 catalytic domain and linker as a carrier protein and/or cbh1 promoter.

In one embodiment of the invention, the culturing is in a medium comprises a protease inhibitor.

In a specific embodiment, the culturing is in a medium comprising one or two protease inhibitors selected from SBTI and chymostatin.

In one embodiment of the method of the invention, the N-glycosylation site occupancy of the produced glycoprotein composition is at least 80%, at least 90%, at least 95%, at least 99%, or 100%.

In one aspect, the invention also relates to a glycoprotein composition obtainable by the method described above.

In one aspect, the invention relates to an antibody composition obtainable by the method described above.

In one embodiment the invention relates to the antibody composition described above, wherein N-glycosylation site occupancy is at least 80%, at least 90%, at least 95%, at least 99%, or 100%.

In one embodiment the invention relates to the antibody composition described above, wherein said antibody composition further comprises, as a major glycoform, either:
i. Manα3[Manα6(Manα3)Manα6]Manβ4GlcNAβ4GlcNAc (Man5 glycoform);
ii. GlcNAcβ2Manα3[Manα6(Manα3)Manα6]Manβ4GlcNAβ4GlcNAc (GlcNAcMan5 glycoform);
iii. Manα6(Manα3)Manβ4GlcNAβ4GlcNAc (Man3 glycoform);
iv. Manα6(GlcNAcβ2Manα3)Manβ4GlcNAβ4GlcNAc (GlcNAcMan3 glycoform); or,
v. complex type N-glycans selected from the G0, G1, or G2 glycoform.

### DESCRIPTION OF THE FIGURES

FIG. 1. Schematic expression cassette design for Leishmania major STT3 targeted to the xylanase 1 locus.

FIG. 2. Example spectra of parental strain M317 (pyr4- of M304) and L. major STT3 clone 26B-a (M421). K means lysine.

FIG. 3. Schematic map of the STT3 expression cassettes.

FIG. 4. Glycan structures produced in Δalg3 strains.

FIG. 5. Normalized protease activity data from culture supernatants from the protease deletion supernatants and the parent strain. Protease activity was measured at pH 5.5 in first 5 strains and at pH 4.5 in the last three deletion strains. Protease activity is against green fluorescent casein. The six protease deletion strain has only 6% of the wild type parent strain and the 7 protease deletion strain protease activity was about 40% less than the 6 protease deletion strain activity.

### DETAILED DESCRIPTION

### Definitions

As used herein, an “expression system” or a “host cell” refers to the cell that is genetically modified to enable the transcription, translation and proper folding of a polypeptide or a protein of interest, typically of mammalian protein.

The term “polynucleotide” or “oligonucleotide” or “nucleic acid” as used herein typically refers to a polymer of at least two nucleotides joined together by a phosphodiester bond and may consist of either ribonucleotides or deoxynucleotides or their derivatives that can be introduced into a host cell for genetic modification of such host cell. For example, a polynucleotide may encode a coding sequence of a protein, and/or comprise control or regulatory sequences of a coding sequence of a protein, such as enhancer or promoter sequences or terminator. A polynucleotide may for example comprise native coding sequence of a gene or their fragments, or variant sequences that have been optimized for optimal gene expression in a specific host cell (for example to take into account codon bias).

As used herein, the term, “optimized” with reference to a polynucleotide means that a polynucleotide has been altered to encode an amino acid sequence using codons that are preferred in the production cell or organism, for example, a filamentous fungal cell such as a Trichoderma cell. Heterologous nucleotide sequences that are transfected in a host cell are typically optimized to retain completely or as much as possible the amino acid sequence originally encoded by the original (not optimized) nucleotide sequence. The optimized sequences herein have been engineered to have codons that are preferred in the corresponding production cell or organism, for example the filamentous fungal cell. The amino acid sequences encoded by optimized nucleotide sequences may also be referred to as optimized.

As used herein, a “peptide” or a “polypeptide” is an amino acid sequence including a plurality of consecutive polymerized amino acid residues. The peptide or polypeptide may include modified amino acid residues, naturally occurring amino acid residues not encoded by a codon, and non-naturally occurring amino acid residues. As used herein, a “protein” may refer to a peptide or a polypeptide or a combination of more than one peptide or polypeptide assembled together by covalent or non-covalent bonds. Unless specified, the term “protein” may encompass one or more amino acid sequences with their post-translation modifications, and in particular with either O-mannosylation or N-glycan modifications.

As used herein, the term “glycoprotein” refers to a protein which comprises at least one N-linked glycan attached to at least one asparagine residue of a protein, or at least one mannose attached to at least one serine or threonine resulting in O-mannosylation. Since glycoproteins as produced in a host cell expression system are usually produced as a mixture of different glycosylation patterns, the terms “glycoprotein” or “glycoprotein composition” encompass the mixtures of glycoproteins as produced by a host cell, with different glycosylation patterns, unless specifically defined.

The terms “N-glycosylation” or “oligosaccharyl transferase activity” are used herein to refer to the covalent linkage of at least an oligosaccharide chain to the side-chain amide nitrogen of asparagine residue (Asn) of a polypeptide.

As used herein, “glycan” refers to an oligosaccharide chain that can be linked to a carrier such as an amino acid, peptide, polypeptide, lipid or a reducing end conjugate. In certain embodiments, the invention relates to N-linked glycans (“N-glycan”) conjugated to a polypeptide N-glycosylation site such as -Asn-Xaa-Ser/Thr- by N-linkage to side-chain amide nitrogen of asparagine residue (Asn), where Xaa is any amino acid residue except Pro. The invention may further relate to glycans as part of dolichol-phospho-oligosaccharide (Dol-P-P-OS) precursor lipid structures, which are precursors of N-linked glycans in the endoplasmic reticulum of eukaryotic cells. The precursor oligosaccharides are linked from their reducing end to two phosphate residues on the dolichol lipid. For example, α3-mannosyltransferase Alg3 modifies the Dol-P-P-oligosaccharide precursor of N-glycans. Generally, the glycan structures described herein are terminal glycan structures, where the non-reducing residues are not modified by other monosaccharide residue or residues.

As used throughout the present disclosure, glycolipid and carbohydrate nomenclature is essentially according to recommendations by the IUPAC-IUB Commission on Biochemical Nomenclature (e.g. Carbohydrate Res. 1998, 312, 167; Carbohydrate Res. 1997, 297, 1; Eur. J. Biochem. 1998, 257, 29). It is assumed that Gal (galactose), Glc (glucose), GlcNAc (N-acetylglucosamine), GalNAc (N-acetylgalactosamine), Man (mannose), and Neu5Ac are of the D-configuration, Fuc of the L-configuration, and all the monosaccharide units in the pyranose form (D-Galp, D-Glcp, D-GlcpNAc, D-GalpNAc, D-Manp, L-Fucp, D-Neup5Ac). The amine group is as defined for natural galactose and glucosamines on the 2-position of GalNAc or GlcNAc. Glycosidic linkages are shown partly in shorter and partly in longer nomenclature, the linkages of the sialic acid SA/Neu5X-residues α3 and α6 mean the same as α2-3 and α2-6, respectively, and for hexose monosaccharide residues α1-3, α1-6, β1-2, β1-3, β1-4, and β1-6 can be shortened as α3, α6, β2, β3, β4, and β6, respectively. Lactosamine refers to type II N-acetyllactosamine, Galβ4GlcNAc, and/or type I N-acetyllactosamine. Galβ3GlcNAc and sialic acid (SA) refer to N-acetylneuraminic acid (Neu5Ac), N-glycolylneuraminic acid (Neu5Gc), or any other natural sialic acid including derivatives of Neu5X. Sialic acid is referred to as NeuNX or Neu5X, where preferably X is Ac or Gc. Occasionally Neu5Ac/Gc/X may be referred to as NeuNAc/NeuNGc/NeuNX.

The sugars typically constituting N-glycans found in mammalian glycoprotein, include, without limitation, N-acetylglucosamine (abbreviated hereafter as “GlcNAc”), mannose (abbreviated hereafter as “Man”), glucose (abbreviated hereafter as “Glc”), galactose (abbreviated hereafter as “Gal”), and sialic acid (abbreviated hereafter as “Neu5Ac”). N-glycans share a common pentasaccharide referred to as the “core” structure Man₃GlcNAc₂ (Manα6(Manα3)Manβ4GlcNAβ4GlcNAc, referred to as Man3).

In some embodiments Man3 glycan or its derivative Manα6(GlcNAcβ2Manα3)Manβ4GlcNAβ4GlcNAc is the major glycoform. When a fucose is attached to the core structure, preferably α6-linked to reducing end GlcNAc, the N-glycan or the core of N-glycan, may be represented as Man₃GlcNAc₂(Fuc). In an embodiment the major N-glycan is Manα3[Manα6(Manα3)Manα6]Manβ4GlcNAβ4GlcNAc (Man5).

Preferred hybrid type N-glycans comprise GlcNAcβ2Manα3[Manα6(Manα3)Manα6]Manβ4GlcNAβ4GlcNAc (“GlcNAcMan5”), or b4-galactosylated derivatives thereof Galβ4GlcNAcMan3, G1, G2, or GalGlcNAcMan5 glycoform.

A “complex N-glycan” refers to a N-glycan which has at least one GlcNAc residue, optionally by GlcNAcβ2-residue, on terminal 1,3 mannose arm of the core structure and at least one GlcNAc residue, optionally by GlcNAcβ2-residue, on terminal 1,6 mannose arm of the core structure.

Such complex N-glycans include, without limitation, GlcNAc₂Man₃GlcNAc₂ (also referred as G0 glycoform), Gal₁GlcNAc₂Man₃GlcNAc₂ (also referred as G1 glycoform), and Gal₂GlcNAc₂Man₃GlcNAc₂ (also referred as G2 glycoform), and their core fucosylated glycoforms FG0, FG1 and FG2, respectively GlcNAc₂Man₃GlcNAc₂(Fuc), Gal₁GlcNAc₂Man₃GlcNAc₂(Fuc), and Gal₂GlcNAc₂Man₃GlcNAc₂(Fuc).

As used herein, the expression “neutral N-glycan” has its general meaning in the art. It refers to non-sialylated N-glycans. In contrast, sialylated N-glycans are acidic.

“Increased” or “Reduced activity of an endogenous enzyme”: The filamentous fungal cell may have increased or reduced levels of activity of various endogenous enzymes. A reduced level of activity may be provided by inhibiting the activity of the endogenous enzyme with an inhibitor, an antibody, or the like. In certain embodiments, the filamentous fungal cell is genetically modified in ways to increase or reduce activity of various endogenous enzymes. “Genetically modified” refers to any recombinant DNA or RNA method used to create a prokaryotic or eukaryotic host cell that expresses a polypeptide at elevated levels, at lowered levels, or in a mutated form. In other words, the host cell has been transfected, transformed, or transduced with a recombinant polynucleotide molecule, and thereby been altered so as to cause the cell to alter expression of a desired protein.

“Genetic modifications” which result in a decrease or deficiency in gene expression, in the function of the gene, or in the function of the gene product (i.e., the protein encoded by the gene) can be referred to as inactivation (complete or partial), knock-out, deletion, disruption, interruption, blockage, silencing, or down-regulation, or attenuation of expression of a gene. For example, a genetic modification in a gene which results in a decrease in the function of the protein encoded by such gene, can be the result of a complete deletion of the gene (i.e., the gene does not exist, and therefore the protein does not exist), a mutation in the gene which results in incomplete (disruption) or no translation of the protein (e.g., the protein is not expressed), or a mutation in the gene which decreases or abolishes the natural function of the protein (e.g., a protein is expressed which has decreased or no enzymatic activity or action). More specifically, reference to decreasing the action of proteins discussed herein generally refers to any genetic modification in the host cell in question, which results in decreased expression and/or functionality (biological activity) of the proteins and includes decreased activity of the proteins (e.g., decreased catalysis), increased inhibition or degradation of the proteins as well as a reduction or elimination of expression of the proteins. For example, the action or activity of a protein can be decreased by blocking or reducing the production of the protein, reducing protein action, or inhibiting the action of the protein. Combinations of some of these modifications are also possible. Blocking or reducing the production of a protein can include placing the gene encoding the protein under the control of a promoter that requires the presence of an inducing compound in the growth medium. By establishing conditions such that the inducer becomes depleted from the medium, the expression of the gene encoding the protein (and therefore, of protein synthesis) could be turned off. Blocking or reducing the action of a protein could also include using an excision technology approach similar to that described in U.S. Pat. No. 4,743,546. To use this approach, the gene encoding the protein of interest is cloned between specific genetic sequences that allow specific, controlled excision of the gene from the genome. Excision could be prompted by, for example, a shift in the cultivation temperature of the culture, as in U.S. Pat. No. 4,743,546, or by some other physical or nutritional signal.

In general, according to the present invention, an increase or a decrease in a given characteristic of a mutant or modified protein (e.g., enzyme activity) is made with reference to the same characteristic of a parent (i.e., normal, not modified) protein that is derived from the same organism (from the same source or parent sequence), which is measured or established under the same or equivalent conditions. Similarly, an increase or decrease in a characteristic of a genetically modified host cell (e.g., expression and/or biological activity of a protein, or production of a product) is made with reference to the same characteristic of a wild-type host cell of the same species, and preferably the same strain, under the same or equivalent conditions. Such conditions include the assay or culture conditions (e.g., medium components, temperature, pH, etc.) under which the activity of the protein (e.g., expression or biological activity) or other characteristic of the host cell is measured, as well as the type of assay used, the host cell that is evaluated, etc. As discussed above, equivalent conditions are conditions (e.g., culture conditions) which are similar, but not necessarily identical (e.g., some conservative changes in conditions can be tolerated), and which do not substantially change the effect on cell growth or enzyme expression or biological activity as compared to a comparison made under the same conditions.

Preferably, a genetically modified host cell that has a genetic modification that increases or decreases (reduces) the activity of a given protein (e.g., a protease) has an increase or decrease, respectively, in the activity or action (e.g., expression, production and/or biological activity) of the protein, as compared to the activity of the protein in a parent host cell (which does not have such genetic modification), of at least about 5%, and more preferably at least about 10%, 15%, 20%, 25%, 30%, 35%, 40%, 45%, 50%, 55 60%, 65%, 70%, 75 80%, 85 90%, 95%, or any percentage, in whole integers between 5% and 100% (e.g., 6%, 7%, 8%, etc.).

In another aspect of the invention, a genetically modified host cell that has a genetic modification that increases or decreases (reduces) the activity of a given protein (e.g., a protease) has an increase or decrease, respectively, in the activity or action (e.g., expression, production and/or biological activity) of the protein, as compared to the activity of the wild-type protein in a parent host cell, of at least about 2-fold, and more preferably at least about 5-fold, 10-fold, 20-fold, 30-fold, 40-fold, 50-fold, 75-fold, 100-fold, 125-fold, 150-fold, or any whole integer increment starting from at least about 2-fold (e.g., 3-fold, 4-fold, 5-fold, 6-fold, etc.).

As used herein, the terms “identical” or “percent identity,” in the context of two or more nucleic acid or amino acid sequences, refers to two or more sequences or subsequences that are the same. Two sequences are “substantially identical” if two sequences have a specified percentage of amino acid residues or nucleotides that are the same (i.e., 29% identity, optionally 30%, 40%, 45%, 50%, 55%, 60%, 65%, 70%, 75%, 80%, 85%, 90%, 95%, 99% or 100% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200, or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. When comparing two sequences for identity, it is not necessary that the sequences be contiguous, but any gap would carry with it a penalty that would reduce the overall percent identity. For blastn, the default parameters are Gap opening penalty=5 and Gap extension penalty=2. For blastp, the default parameters are Gap opening penalty=11 and Gap extension penalty=1.

A “comparison window,” as used herein, includes reference to a segment of any one of the number of contiguous positions including, but not limited to from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1981), by the homology alignment algorithm of Needleman and Wunsch (1970) J Mol Biol 48(3):443-453, by the search for similarity method of Pearson and Lipman (1988) Proc Natl Acad Sci USA 85(8):2444-2448, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection [see, e.g., Brent et al., (2003) Current Protocols in Molecular Biology, John Wiley & Sons, Inc. (Ringbou Ed)].

Two examples of algorithms that are suitable for determining percent sequence identity and sequence similarity are the BLAST and BLAST 2.0 algorithms, which are described in Altschul et al. (1997) Nucleic Acids Res 25(17):3389-3402 and Altschul et al. (1990) J. Mol Biol 215(3)-403-410, respectively. Software for performing BLAST analyses is publicly available through the National Center for Biotechnology Information. The BLASTN program (for nucleotide sequences) uses as defaults a word length (W) of 11, an expectation (E) or 10, M=5, N=−4, and a comparison of both strands. For amino acid sequences, the BLASTP program uses as defaults a word length of 3, and expectation (E) of 10, and the BLOSUM62 scoring matrix [see Henikoff and Henikoff, (1992) Proc Natl Acad Sci USA 89(22)10915-10919] alignments (B) of 50, expectation (E) of 10, M=5, N=−4, and a comparison of both strands.

The BLAST algorithm also performs a statistical analysis of the similarity between two sequences (see, e.g., Karlin and Altschul, (1993) Proc Natl Acad Sci USA 90(12):5873-5877). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

“Functional variant” or “functional homologous gene” as used herein refers to a coding sequence or a protein having sequence similarity with a reference sequence, typically, at least 30%, 40%, 50%, 60%, 70%, 80%, 90% or 95% identity with the reference coding sequence or protein, and retaining substantially the same function as said reference coding sequence or protein. A functional variant may retain the same function but with reduced or increased activity. Functional variants include natural variants, for example, homologs from different species or artificial variants, resulting from the introduction of a mutation in the coding sequence. Functional variant may be a variant with only conservatively modified mutations.

“Conservatively modified mutations” as used herein include individual substitutions, deletions or additions to an encoded amino acid sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Filamentous Fungal Cells

As used herein, “filamentous fungal cells” include cells from all filamentous forms of the subdivision Eumycota and Oomycota (as defined by Hawksworth et al., In, Ainsworth and Bisby's Dictionary of The Fungi, 8th edition, 1995, CAB International, University Press, Cambridge, UK). Filamentous fungal cells are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

Preferably, the filamentous fungal cell is not adversely affected by the transduction of the necessary nucleic acid sequences, the subsequent expression of the proteins (e.g., mammalian proteins), or the resulting intermediates. General methods to disrupt genes of and cultivate filamentous fungal cells are disclosed, for example, for Penicillium, in Kopke et al. (2010) Appl Environ Microbiol. 76(14):4664-74. doi: 10.1128/AEM.00670-10, for Aspergillus, in Maruyama and Kitamoto (2011), Methods in Molecular Biology, vol. 765, D0110.1007/978-1-61779-197-0_27; for Neurospora, in Collopy et al. (2010) Methods Mol Biol. 2010; 638:33-40. doi: 10.1007/978-1-60761-611-5_3; and for Myceliophthora or Chrysosporium PCT/NL2010/000045 and PCT/EP98/06496.

Examples of suitable filamentous fungal cells include, without limitation, cells from an Acremonium, Aspergillus, Fusarium, Humicola, Mucor, Myceliophthora, Neurospora, Penicillium, Scytalidium, Thielavia, Tolypocladium, or Trichoderma/Hypocrea strain.

In certain embodiments, the filamentous fungal cell is from a Trichoderma sp., Acremonium, Aspergillus, Aureobasidium, Cryptococcus, Chrysosporium, Chrysosporium lucknowense, Filibasidium, Fusarium, Gibberella, Magnaporthe, Mucor, Myceliophthora, Myrothecium, Neocallimastix, Neurospora, Paecilomyces, Penicillium, Piromyces, Schizophyllum, Talaromyces, Thermoascus, Thielavia, or Tolypocladium strain.

In some embodiments, the filamentous fungal cell is a Myceliophthora or Chrysosporium, Neurospora, Aspergillus, Fusarium or Trichoderma strain.

Aspergillus fungal cells of the present disclosure may include, without limitation, Aspergillus aculeatus, Aspergillus awamori, Aspergillus clavatus, Aspergillus flavus, Aspergillus foetidus, Aspergillus fumigatus, Aspergillus japonicus, Aspergillus nidulans, Aspergillus niger, Aspergillus oryzae, or Aspergillus terreus.

Neurospora fungal cells of the present disclosure may include, without limitation, Neurospora crassa.

Myceliophthora fungal cells of the present disclosure may include, without limitation, Myceliophthora thermophila.

In a preferred embodiment, the filamentous fungal cell is a Trichoderma fungal cell. Trichoderma fungal cells of the present disclosure may be derived from a wild-type Trichoderma strain or a mutant thereof. Examples of suitable Trichoderma fungal cells include, without limitation, Trichoderma harzianum, Trichoderma koningii, Trichoderma longibrachiatum, Trichoderma reesei, Trichoderma atroviride, Trichoderma virens, Trichoderma viride; and alternative sexual form thereof (i.e., Hypocrea).

In a more preferred embodiment, the filamentous fungal cell is a Trichoderma reesei, and for example, strains derived from ATCC 13631 (QM 6a), ATCC 24449 (radiation mutant 207 of QM 6a), ATCC 26921 (QM 9414; mutant of ATCC 24449), VTT-D-00775 (Selinheimo et al., FEBS J., 2006, 273: 4322-4335), Rut-C30 (ATCC 56765), RL-P37 (NRRL 15709) or T. harzianum isolate T3 (Wolffhechel, H., 1989).

The invention described herein relates to a filamentous fungal cell, for example selected from Trichoderma, Neurospora, Myceliophthora or a Chrysosporium cells, such as Trichoderma reesei fungal cell, comprising:
i. one or more mutation that reduces or eliminates one or more endogenous protease activity compared to a parental filamentous fungal cell which does not have said mutation(s),
ii. a polynucleotide encoding a heterologous catalytic subunit of oligosaccharyl transferase, and
iii. a polynucleotide encoding a heterologous glycoprotein, wherein said catalytic subunit of oligosaccharyl transferase is selected from Leishmania oligosaccharyl transferase catalytic subunits.

Proteases with Reduced Activity

It has been found that reducing protease activity enables to increase substantially the production of heterologous mammalian protein. Indeed, such proteases found in filamentous fungal cells that express a heterologous protein normally catalyse significant degradation of the expressed recombinant protein. Thus, by reducing the activity of proteases in filamentous fungal cells that express a heterologous protein, the stability of the expressed protein is increased, resulting in an increased level of production of the protein, and in some circumstances, improved quality of the produced protein (e.g., full-length instead of degraded).

Proteases include, without limitation, aspartic proteases, trypsin-like serine proteases, subtilisin proteases, glutamic proteases, and sedolisin proteases. Such proteases may be identified and isolated from filamentous fungal cells and tested to determine whether reduction in their activity affects the production of a recombinant polypeptide from the filamentous fungal cell. Methods for identifying and isolating proteases are well known in the art, and include, without limitation, affinity chromatography, zymogram assays, and gel electrophoresis. An identified protease may then be tested by deleting the gene encoding the identified protease from a filamentous fungal cell that expresses a recombinant polypeptide, such a heterologous or mammalian polypeptide, and determining whether the deletion results in a decrease in total protease activity of the cell, and an increase in the level of production of the expressed recombinant polypeptide. Methods for deleting genes, measuring total protease activity, and measuring levels of produced protein are well known in the art and include the methods described herein.

Aspartic Proteases

Aspartic proteases are enzymes that use an aspartate residue for hydrolysis of the peptide bonds in polypeptides and proteins. Typically, aspartic proteases contain two highly-conserved aspartate residues in their active site which are optimally active at acidic pH. Aspartic proteases from eukaryotic organisms such as Trichoderma fungi include pepsins, cathepsins, and renins. Such aspartic proteases have a two-domain structure, which is thought to arise from ancestral gene duplication. Consistent with such a duplication event, the overall fold of each domain is similar, though the sequences of the two domains have begun to diverge. Each domain contributes one of the catalytic aspartate residues. The active site is in a cleft formed by the two domains of the aspartic proteases. Eukaryotic aspartic proteases further include conserved disulfide bridges, which can assist in identification of the polypeptides as being aspartic acid proteases.

Ten aspartic proteases have been identified in Trichoderma fungal cells: pep1 (tre74156); pep2 (tre53961); pep3 (tre121133); pep4 (tre77579), pep5 (tre81004), and pep7 (tre58669), pep8 (tre122076), pep9 (tre79807), pep11 (121306), and pep12 (tre119876).

Examples of suitable aspartic proteases include, without limitation, Trichoderma reesei pep1 (SEQ ID NO: 22), Trichoderma reesei pep2 (SEQ ID NO: 18), Trichoderma reesei pep3 (SEQ ID NO: 19); Trichoderma reesei pep4 (SEQ ID NO: 20), Trichoderma reesei pep5 (SEQ ID NO: 21) and Trichoderma reesei pep7 (SEQ ID NO:23), Trichoderma reesei EGR48424 pep8 (SEQ ID NO:85), Trichoderma reesei pep9 (SEQ ID NO:87), Trichoderma reesei EGR49498 pep11 (SEQ ID NO:86), Trichoderma reesei EGR52517 pep12 (SEQ ID NO:35), and homologs thereof. Examples of homologs of pep1, pep2, pep3, pep4, pep5, pep7, pep8, pep11 and pep12 proteases identified in other organisms are also described in PCT/EP/2013/050186, the content of which being incorporated by reference.

Trypsin-Like Serine Proteases

Trypsin-like serine proteases are enzymes with substrate specificity similar to that of trypsin. Trypsin-like serine proteases use a serine residue for hydrolysis of the peptide bonds in polypeptides and proteins. Typically, trypsin-like serine proteases cleave peptide bonds following a positively-charged amino acid residue. Trypsin-like serine proteases from eukaryotic organisms such as Trichoderma fungi include trypsin 1, trypsin 2, and mesotrypsin. Such trypsin-like serine proteases generally contain a catalytic triad of three amino acid residues (such as histidine, aspartate, and serine) that form a charge relay that serves to make the active site serine nucleophilic. Eukaryotic trypsin-like serine proteases further include an “oxyanion hole” formed by the backbone amide hydrogen atoms of glycine and serine, which can assist in identification of the polypeptides as being trypsin-like serine proteases.

One trypsin-like serine protease has been identified in Trichoderma fungal cells: tsp1 (tre73897). As discussed in PCT/EP/2013/050186, tsp1 has been demonstrated to have a significant impact on expression of recombinant glycoproteins, such as immunoglobulins.

Examples of suitable tsp1 proteases include, without limitation, Trichoderma reesei tsp1 (SEQ ID NO: 24) and homologs thereof. Examples of homologs of tsp1 proteases identified in other organisms are described in PCT/EP/2013/050186.

Subtilisin Proteases

Subtilisin proteases are enzymes with substrate specificity similar to that of subtilisin. Subtilisin proteases use a serine residue for hydrolysis of the peptide bonds in polypeptides and proteins. Generally, subtilisin proteases are serine proteases that contain a catalytic triad of the three amino acids aspartate, histidine, and serine. The arrangement of these catalytic residues is shared with the prototypical subtilisin from Bacillus licheniformis. Subtilisin proteases from eukaryotic organisms such as Trichoderma fungi include furin, MBTPS1, and TPP2. Eukaryotic trypsin-like serine proteases further include an aspartic acid residue in the oxyanion hole.

Seven subtilisin proteases have been identified in Trichoderma fungal cells: slp1 (tre51365); slp2 (tre123244); slp3 (tre123234); slp5 (tre64719), slp6 (tre121495), slp7 (tre123865), and slp8 (tre58698). Subtilisin protease slp7 resembles also sedolisin protease tpp1.

Examples of suitable slp proteases include, without limitation, Trichoderma reesei slp1 (SEQ ID NO: 25), slp2 (SEQ ID NO: 26); slp3 (SEQ ID NO: 27); slp5 (SEQ ID NO: 28), slp6 (SEQ ID NO: 29), slp7 (SEQ ID NO: 30), and slp8 (SEQ ID NO: 31), and homologs thereof. Examples of homologs of slp proteases identified in other organisms are described in in PCT/EP/2013/050186.

Glutamic Proteases

Glutamic proteases are enzymes that hydrolyse the peptide bonds in polypeptides and proteins. Glutamic proteases are insensitive to pepstatin A, and so are sometimes referred to as pepstatin insensitive acid proteases. While glutamic proteases were previously grouped with the aspartic proteases and often jointly referred to as acid proteases, it has been recently found that glutamic proteases have very different active site residues than aspartic proteases.

Two glutamic proteases have been identified in Trichoderma fungal cells: gap1 (tre69555) and gap2 (tre106661).

Examples of suitable gap proteases include, without limitation, Trichoderma reesei gap1 (SEQ ID NO: 32), Trichoderma reesei gap2 (SEQ ID NO: 33), and homologs thereof. Examples of homologs of gap proteases identified in other organisms are described in PCT/EP/2013/050186.

Sedolisin Proteases and Homologs of Proteases

Sedolisin proteases are enzymes that use a serine residue for hydrolysis of the peptide bonds in polypeptides and proteins. Sedolisin proteases generally contain a unique catalytic triad of serine, glutamate, and aspartate. Sedolisin proteases also contain an aspartate residue in the oxyanion hole. Sedolisin proteases from eukaryotic organisms such as Trichoderma fungi include tripeptidyl peptidase.

Examples of suitable tpp1 proteases include, without limitation, Trichoderma reesei tpp1 tre82623 (SEQ ID NO: 34) and homologs thereof. Examples of homologs of tpp1 proteases identified in other organisms are described in PCT/EP/2013/050186.

As used in reference to protease, the term “homolog” refers to a protein which has protease activity and exhibit sequence similarity with a known (reference) protease sequence. Homologs may be identified by any method known in the art, preferably, by using the BLAST tool to compare a reference sequence to a single second sequence or fragment of a sequence or to a database of sequences. As described in the “Definitions” section, BLAST will compare sequences based upon percent identity and similarity.

Preferably, a homologous protease has at least 30% identity with (optionally 30%, 40%, 45%, 50%, 55%, 60%, 65%, 70%, 75%, 80%, 85%, 90%, 95%, 99% or 100% identity over a specified region, or, when not specified, over the entire sequence), when compared to one of the protease sequences listed above, including T. reesei pep1, pep2, pep3, pep4, pep5, pep7, pep8, pep9, pep11, pep12, tsp1, slp1, slp2, slp3, slp5, slp6, slp7, slp8, tpp1, gap1 and gap2. Corresponding homologous proteases from N. crassa and M. thermophila are shown in SEQ ID NO: 136-169.

Reducing the Activity of Proteases in the Filamentous Fungal Cell of the Invention

The filamentous fungal cells according to the invention have reduced activity of at least one endogenous protease, typically 2, 3, 4, 5 or more, in order to improve the stability and production of the protein with increased N-glycosylation site occupancy in said filamentous fungal cell, preferably in a Trichoderma cell.

Total protease activity can be measured according to standard methods in the art and, for example, as described herein using protease assay kit (QuantiCleave protease assay kit, Pierce #23263) with succinylated casein as substrate.

The activity of proteases found in filamentous fungal cells can be reduced by any method known to those of skill in the art. In some embodiments reduced activity of proteases is achieved by reducing the expression of the protease, for example, by promoter modification or RNAi.

In further embodiments, the reduced or eliminated expression of the proteases is the result of anti-sense polynucleotides or RNAi constructs that are specific for each of the genes encoding each of the proteases. In one embodiment, an RNAi construct is specific for a gene encoding an aspartic protease such as a pep-type protease, a trypsin-like serine proteases such as a tsp1, a glutamic protease such as a gap-type protease, a subtilisin protease such as a slp-type protease, or a sedolisin protease such as a tpp1 or a slp7 protease. In one embodiment, an RNAi construct is specific for the gene encoding a slp-type protease. In one embodiment, an RNAi construct is specific for the gene encoding slp2, slp3, slp5 or slp6. In one embodiment, an RNAi construct is specific for two or more proteases. In one embodiment, two or more proteases are any one of the pep-type proteases, any one of the trypsin-like serine proteasess, any one of the slp-type proteases, any one of the gap-type proteases and/or any one of the sedolisin proteases. In one embodiment, two or more proteases are slp2, slp3, slp5 and/or slp6. In one embodiment, RNAi construct comprises any one of the following nucleic acid sequences (see also PCT/EP/2013/050186).

RNAi Target sequence
(SEQ ID NO: 15)
GCACACTTTCAAGATTGGC
(SEQ ID NO: 16)
GTACGGTGTTGCCAAGAAG
(SEQ ID NO: 17)
GTTGAGTACATCGAGCGCGACAGCATTGTGCACACCATGCTTCCCCTCGA
GTCCAAGGACAGCATCATCGTTGAGGACTCGTGCAACGGCGAGACGGAGA
AGCAGGCTCCCTGGGGTCTTGCCCGTATCTCTCACCGAGAGACGCTCAAC
TTTGGCTCCTTCAACAAGTACCTCTACACCGCTGATGGTGGTGAGGGTGT
TGATGCCTATGTCATTGACACCGGCACCAACATCGAGCACGTCGACTTTG
AGGGTCGTGCCAAGTGGGGCAAGACCATCCCTGCCGGCGATGAGGACGAG
GACGGCAACGGCCACGGCACTCACTGCTCTGGTACCGTTGCTGGTAAGAA
GTACGGTGTTGCCAAGAAGGCCCACGTCTACGCCGTCAAGGTGCTCCGAT
CCAACGGATCCGGCACCATGTCTGACGTCGTCAAGGGCGTCGAGTACG

In other embodiments, reduced activity of proteases is achieved by modifying the gene encoding the protease. Examples of such modifications include, without limitation, a mutation, such as a deletion or disruption of the gene encoding said endogenous protease activity.

Accordingly, the invention relates to a filamentous fungal cell, such as a Trichoderma cell, which has a mutation that reduces or eliminates at least one endogenous protease activity compared to a parental filamentous fungal cell which does not have such protease deficient mutation, said filamentous fungal cell further comprising a polynucleotide encoding a heterologous catalytic subunit of oligosaccharyl transferase from Leishmania.

Deletion or disruption mutation includes without limitation knock-out mutation, a truncation mutation, a point mutation, a missense mutation, a substitution mutation, a frameshift mutation, an insertion mutation, a duplication mutation, an amplification mutation, a translocation mutation, or an inversion mutation, and that results in a reduction in the corresponding protease activity. Methods of generating at least one mutation in a protease encoding gene of interest are well known in the art and include, without limitation, random mutagenesis and screening, site-directed mutagenesis, PCR mutagenesis, insertional mutagenesis, chemical mutagenesis, and irradiation.

In certain embodiments, a portion of the protease encoding gene is modified, such as the region encoding the catalytic domain, the coding region, or a control sequence required for expression of the coding region. Such a control sequence of the gene may be a promoter sequence or a functional part thereof, i.e., a part that is sufficient for affecting expression of the gene. For example, a promoter sequence may be inactivated resulting in no expression or a weaker promoter may be substituted for the native promoter sequence to reduce expression of the coding sequence. Other control sequences for possible modification include, without limitation, a leader sequence, a propeptide sequence, a signal sequence, a transcription terminator, and a transcriptional activator.

Protease encoding genes that are present in filamentous fungal cells may also be modified by utilizing gene deletion techniques to eliminate or reduce expression of the gene. Gene deletion techniques enable the partial or complete removal of the gene thereby eliminating their expression. In such methods, deletion of the gene may be accomplished by homologous recombination using a plasmid that has been constructed to contiguously contain the 5′ and 3′ regions flanking the gene.

The protease encoding genes that are present in filamentous fungal cells may also be modified by introducing, substituting, and/or removing one or more nucleotides in the gene, or a control sequence thereof required for the transcription or translation of the gene. For example, nucleotides may be inserted or removed for the introduction of a stop codon, the removal of the start codon, or a frame-shift of the open reading frame. Such a modification may be accomplished by methods known in the art, including without limitation, site-directed mutagenesis and peR generated mutagenesis (see, for example, Botstein and Shortie, 1985, Science 229: 4719; Lo et al., 1985, Proceedings of the National Academy of Sciences USA 81: 2285; Higuchi et al., 1988, Nucleic Acids Research 16: 7351; Shimada, 1996, Meth. Mol. Bioi. 57: 157; Ho et al., 1989, Gene 77: 61; Horton et al., 1989, Gene 77: 61; and Sarkar and Sommer, 1990, BioTechniques 8: 404).

Additionally, protease encoding genes that are present in filamentous fungal cells may be modified by gene disruption techniques by inserting into the gene a disruptive nucleic acid construct containing a nucleic acid fragment homologous to the gene that will create a duplication of the region of homology and incorporate construct nucleic acid between the duplicated regions. Such a gene disruption can eliminate gene expression if the inserted construct separates the promoter of the gene from the coding region or interrupts the coding sequence such that a nonfunctional gene product results. A disrupting construct may be simply a selectable marker gene accompanied by 5′ and 3′ regions homologous to the gene. The selectable marker enables identification of transformants containing the disrupted gene.

Protease encoding genes that are present in filamentous fungal cells may also be modified by the process of gene conversion (see, for example, Iglesias and Trautner, 1983, Molecular General Genetics 189:5 73-76). For example, in the gene conversion a nucleotide sequence corresponding to the gene is mutagenized in vitro to produce a defective nucleotide sequence, which is then transformed into a Trichoderma strain to produce a defective gene. By homologous recombination, the defective nucleotide sequence replaces the endogenous gene. It may be desirable that the defective nucleotide sequence also contains a marker for selection of transformants containing the defective gene.

Protease encoding genes of the present disclosure that are present in filamentous fungal cells that express a recombinant polypeptide may also be modified by established anti-sense techniques using a nucleotide sequence complementary to the nucleotide sequence of the gene (see, for example, Parish and Stoker, 1997, FEMS Microbiology Letters 154: 151-157). In particular, expression of the gene by filamentous fungal cells may be reduced or inactivated by introducing a nucleotide sequence complementary to the nucleotide sequence of the gene, which may be transcribed in the strain and is capable of hybridizing to the mRNA produced in the cells. Under conditions allowing the complementary anti-sense nucleotide sequence to hybridize to the mRNA, the amount of protein translated is thus reduced or eliminated.

Protease encoding genes that are present in filamentous fungal cells may also be modified by random or specific mutagenesis using methods well known in the art, including without limitation, chemical mutagenesis (see, for example, Hopwood, The Isolation of Mutants in Methods in Microbiology (J. R. Norris and D. W. Ribbons, eds.) pp. 363-433, Academic Press, New York, 25 1970). Modification of the gene may be performed by subjecting filamentous fungal cells to mutagenesis and screening for mutant cells in which expression of the gene has been reduced or inactivated. The mutagenesis, which may be specific or random, may be performed, for example, by use of a suitable physical or chemical mutagenizing agent, use of a suitable oligonucleotide, subjecting the DNA sequence to peR generated mutagenesis, or any combination thereof. Examples of physical and chemical mutagenizing agents include, without limitation, ultraviolet (UV) irradiation, hydroxylamine, N-methyl-N′-nitro-N-nitrosoguanidine (MNNG), N-methyl-N′-nitrosogaunidine (NTG) O-methyl hydroxylamine, nitrous acid, ethyl methane sulphonate (EMS), sodium bisulphite, formic acid, and nucleotide analogues. When such agents are used, the mutagenesis is typically performed by incubating the filamentous fungal cells, such as Trichoderma cells, to be mutagenized in the presence of the mutagenizing agent of choice under suitable conditions, and then selecting for mutants exhibiting reduced or no expression of the gene.

In certain embodiments, the at least one mutation or modification in a protease encoding gene of the present disclosure results in a modified protease that has no detectable protease activity. In other embodiments, the at least one modification in a protease encoding gene of the present disclosure results in a modified protease that has at least 25% less, at least 50% less, at least 75% less, at least 90%, at least 95%, or a higher percentage less protease activity compared to a corresponding non-modified protease.

The filamentous fungal cells or Trichoderma fungal cells of the present disclosure may have reduced or no detectable protease activity of at least three, or at least four proteases selected from the group consisting of pep1, pep2, pep3, pep4, pep5, pep8, pep9, pep11, pep12, tsp1, slp1, slp2, slp3, slp5, slp6, slp7, gap1 and gap2. In preferred embodiment, a filamentous fungal cell according to the invention is a filamentous fungal cell which has a deletion or disruption in at least 3 or 4 endogenous proteases, resulting in no detectable activity for such deleted or disrupted endogenous proteases and further comprising a polynucleotide encoding a heterologous catalytic subunit of oligosaccharyl transferase from Leishmania.

In certain embodiments, the filamentous fungal cell or Trichoderma cell, has reduced or no detectable protease activity in pep1, tsp1, and slp1. In other embodiments, the filamentous fungal cell or Trichoderma cell, has reduced or no detectable protease activity in gap1, slp1, and pep1. In certain embodiments, the filamentous fungal cell or Trichoderma cell, has reduced or no detectable protease activity in slp2, pep1 and gap1. In certain embodiments, the filamentous fungal cell or Trichoderma cell, has reduced or no detectable protease activity in slp2, pep1, gap1 and pep4. In certain embodiments, the filamentous fungal cell or Trichoderma cell, has reduced or no detectable protease activity in slp2, pep1, gap1, pep4 and slp1. In certain embodiments, the filamentous fungal cell or Trichoderma cell, has reduced or no detectable protease activity in slp2, pep1, gap1, pep4, slp1, and slp3. In certain embodiments, the filamentous fungal cell or Trichoderma cell, has reduced or no detectable protease activity in slp2, pep1, gap1, pep4, slp1, slp3, and pep3. In certain embodiments, the filamentous fungal cell or Trichoderma cell, has reduced or no detectable protease activity in slp2, pep1, gap1, pep4, slp1, slp3, pep3 and pep2. In certain embodiments, the filamentous fungal cell or Trichoderma cell, has reduced or no detectable protease activity in slp2, pep1, gap1, pep4, slp1, slp3, pep3, pep2 and pep5. In certain embodiments, the filamentous fungal cell or Trichoderma cell, has reduced or no detectable protease activity in slp2, pep1, gap1, pep4, slp1, slp3, pep3, pep2, pep5 and tsp1. In certain embodiments, the filamentous fungal cell or Trichoderma cell, has reduced or no detectable protease activity in slp2, pep1, gap1, pep4, slp1, slp3, pep3, pep2, pep5, tsp1 and slp7. In certain embodiments, the filamentous fungal cell or Trichoderma cell, has reduced or no detectable protease activity in slp2, pep1, gap1, pep4, slp1, slp3, pep3, pep2, pep5, tsp1, slp7 and slp8. In certain embodiments, the filamentous fungal cell or Trichoderma cell, has reduced or no detectable protease activity in slp2, pep1, gap1, pep4, slp1, slp3, pep3, pep2, pep5, tsp1, slp7, slp8 and gap2. In certain embodiments, the filamentous fungal cell or Trichoderma cell, has reduced or no detectable protease activity in at least three endogenous proteases selected from the group consisting of pep1, pep2, pep3, pep4, pep5, pep8, pep9, pep11, pep12, tsp1, slp2, slp3, slp7, gap1 and gap2. In certain embodiments, the filamentous fungal cell or Trichoderma cell, has reduced or no detectable protease activity in at least three to six endogenous proteases selected from the group consisting of pep1, pep2, pep3, pep4, pep5, tsp1, slp1, slp2, slp3, gap1 and gap2. In certain embodiments, the filamentous fungal cell or Trichoderma cell, has reduced or no detectable protease activity in at least seven to ten endogenous proteases selected from the group consisting of pep1, pep2, pep3, pep4, pep5, pep7, pep8, tsp1, slp1, slp2, slp3, slp5, slp6, slp7, slp8, tpp1, gap1 and gap2.

Expression of Heterologous Catalytic Subunits of Oligosaccharyl Transferase in Filamentous Fungal Cells

As used herein, the expression “oligosaccharyl transferase” or OST refers to the enzymatic complex that transfers a 14-sugar oligosaccharide from dolichol to nascent protein. It is a type of glycosyltransferase. The sugar Glc3Man9GlcNAc2 is attached to an asparagine (Asn) residue in the sequence Asn-X-Ser or Asn-X-Thr where X is any amino acid except proline. This sequence is called a glycosylation sequon. The reaction catalyzed by OST is the central step in the N-linked glycosylation pathway.

In most eukaryotes, OST is a hetero-oligomeric complex composed of eight different proteins, in which the STT3 component is believed to be the catalytic subunit.

According to the present invention, the heterologous catalytic subunit of oligosaccharyl transferase is selected from Leishmania oligosaccharyl transferase catalytic subunits. There are four STT3 paralogues in the parasitic protozoa Leishmania, named STT3A, STT3B, STT3C and STT3D.

In one embodiment, the heterologous catalytic subunit of oligosaccharyl transferase is STT3D from Leishmania major (having the amino acid sequence as set forth in SEQ ID No:1).

In another embodiment, the heterologous catalytic subunit of oligosaccharyl transferase is STT3D from Leishmania infantum (having the amino acid sequence as set forth in SEQ ID No:8).

In another embodiment, the heterologous catalytic subunit of oligosaccharyl transferase is STT3D from Leishmania braziliensis (having the amino acid sequence as set forth in SEQ ID No:89).

In another embodiment, the heterologous catalytic subunit of oligosaccharyl transferase is STT3D from Leishmania mexicana (having the amino acid sequence as set forth in SEQ ID No:91).

In yet another embodiment, the heterologous catalytic subunit of oligosaccharyl transferase is a functional variant polypeptide having at least 50%, preferably at least 60%, even more preferably at least 70%, 80%, 90%, 95% identity with SEQ ID NO:1, SEQ ID NO:8, SEQ ID NO: 89 or SEQ ID NO: 91.

In yet another embodiment, the heterologous catalytic subunit of oligosaccharyl transferase is a functional variant polypeptide having at least 50%, preferably at least 60%, even more preferably at least 70%, 80%, 90%, 95% identity with SEQ ID NO:1 or SEQ ID NO:8.

In one embodiment of the invention, the polynucleotide encoding heterologous catalytic subunit of oligosaccharyl transferase comprises SEQ ID NO:2.

SEQ ID NO:2 is a codon-optimized version of the STT3D gene from L. major (gi389594572|XM_003722461.1).

In one embodiment of the invention, the polynucleotide encoding heterologous catalytic subunit of oligosaccharyl transferase comprises SEQ ID NO:9.

SEQ ID NO:9 is a codon-optimized version of the STT3D gene from L. major (gi339899220|XM_003392747.1D.

In one embodiment of the invention, the polynucleotide encoding heterologous catalytic subunit of oligosaccharyl transferase comprises SEQ ID NO:88 or a variant or SEQ ID NO: 88 which has been codon-optimized for expression in filamentous fungal cells such as Trichoderma reesei.

In one embodiment of the invention, the polynucleotide encoding heterologous catalytic subunit of oligosaccharyl transferase comprises SEQ ID NO:90 or a variant or SEQ ID NO: 90 which has been codon-optimized for expression in filamentous fungal cells such as Trichoderma reesei.

In one embodiment of the invention, the polynucleotide encoding a heterologous catalytic subunit of oligosaccharyl transferase comprises a polynucleotide encoding a functional variant polypeptide of STT3D from Leishmania major, Leishmania infantum, Leishmania braziliens or Leishmania mexicana having at least 50%, preferably at least 60%, even more preferably at least 70%, 80%, 90%, 95% identity with SEQ ID NO:1, SEQ ID NO:8, SEQ ID NO: 89 or SEQ ID NO: 91.

In one embodiment of the invention, the polynucleotide encoding a heterologous catalytic subunit of oligosaccharyl transferase comprises a polynucleotide encoding a functional variant polypeptide of STT3D from Leishmania major or Leishmania infantum having at least 50%, preferably at least 60%, even more preferably at least 70%, 80%, 90%, 95% identity with SEQ ID NO:1 or SEQ ID NO:8.

In one embodiment of the invention, the polynucleotide encoding a heterologous catalytic subunit of oligosaccharyl transferase is under the control of a promoter for the constitutive expression of said oligosaccharyl transferase is said filamentous fungal cell.

Promoters that may be used for expression of the oligosaccharyl transferase include constitutive promoters such as gpd or cDNA1, promoters of endogenous glycosylation enzymes and glycosyltransferases such as mannosyltransferases that synthesize N-glycans in the Golgi or ER, and inducible promoters of high-yield endogenous proteins such as the cbh1 promoter.

In one embodiment of the invention, said promoter is the cDNA1 promoter from Trichoderma reesei.

Increasing N-Glycosylation Site Occupancy in Filamentous Fungal Cell of the Invention

The filamentous fungal cells according to the invention have increased oligosaccharide transferase activity, in order to increase N-glycosylation site occupancy.

The N-glycosylation site occupancy can be measured by standard methods in the art (for example, Schulz and Aebi (2009) Analysis of Glycosylation Site Occupancy Reveals a Role for Ost3p and Ost6p in Site-specific N-Glycosylation Efficiency, Molecular & Cellular Proteomics, 8:357-364, or Millward et al. (2008), Effect of constant and variable domain glycosylation on pharmacokinetics of therapeutic antibodies in mice, Biologicals, 36:41-47, Forno et al. (2004)N- and O-linked carbohydrates and glycosylation site occupancy in recombinant human granulocyte-macrophage colony-stimulating factor secreted by a Chinese hamster ovary cell line, Eur. J. Biochem. 271: 907-919) or methods as described herein in the Examples.

The N-glycosylation site occupancy refers to the molar percentage (or mol %) of the heterologous glycoproteins that are N-glycosylated with respect to the total number of heterologous glycoprotein produced by the filamentous fungal cell (as described in Example 1 below).

In one embodiment of the invention, the N-glycosylation site occupancy is at least 95%, and Man3, Man5, G0, G1 and/or G2 glycoforms represent at least 50% of total neutral N-glycans of the heterologous glycoprotein.

The percentage of various glycoforms with respect to the total neutral N-glycans of the heterologous glycoprotein can be measured for example as described in WO2012069593.

In an embodiment, the heterologous protein with increased N-glycosylation site occupancy is selected from the group consisting of:
a) an immunoglobulin, such as IgG,
b) a light chain or heavy chain of an immunoglobulin,
c) a heavy chain or a light chain of an antibody,
d) a single chain antibody,
e) a camelid antibody,
f) a monomeric or multimeric single domain antibody,
g) a FAb-fragment, a FAb2-fragment, and,
h) their antigen-binding fragments.

Methods for Producing Glycoproteins with Increased N-Glycosylation Site Occupancy and Mammalian-Like N-Glycans

The filamentous fungal cells according to the present invention may be useful in particular for producing heterologous glycoprotein composition, such as antibody composition, with increased N-glycosylation site occupancy and mammalian-like N-glycans, such as complex N-glycans.

Accordingly, in one aspect, the filamentous fungal cell is further genetically modified to produce a mammalian-like N-glycan, thereby enabling in vivo production of glycoprotein or antibody composition with increased N-glycosylation site occupancy and with mammalian-like N-glycan as major glycoforms of said glycoprotein or antibody.

In certain embodiments, this aspect includes methods of producing glycoproteins or antibodies with mammalian-like N-glycans in a Trichoderma cell.

In certain embodiment, the glycoprotein or antibody comprises, as a major glycoform, the mammalian-like N-glycan having the formula [{Galβ4}ₓGlcNAcβ2]_{z}Manα3([{Galβ4}_{y}GlcNAcβ2]_{w}Manα6)Manβ4GlcNAcβ[Fucα6]ₐGlcNAc, where ( ) defines a branch in the structure, where [ ] or { } define a part of the glycan structure either present or absent in a linear sequence, and where a, x, y, z and w are 0 or 1, independently. In an embodiment w and z are 1, and x and y are 0 for a non-galactosylated G0 structure; both x and y are 1 for a G2 structure; and only either one of x or y is 1 for a G1 structure. When a is 1, the structure is core fucosylated such as a FG0, FG1 or FG2 glycan.

In certain embodiments, the glycoprotein or antibody comprises, as a major glycoform, mammalian-like N-glycan selected from the group consisting of:
i. Manα3[Manα6(Manα3)Manα6]Manβ4GlcNAβ4GlcNAc (Man5 glycoform);
ii. GlcNAcβ2Manα3[Manα6(Manα3)Manα6]Manβ4GlcNAβ4GlcNAc (GlcNAcMan5 glycoform);
iii. Manα6(Manα3)Manβ4GlcNAβ4GlcNAc (Man3 glycoform);
iv. Manα6(GlcNAcβ2Manα3)Manβ4GlcNAβ4GlcNAc (GlcNAcMan3) or,
v. complex type N-glycans selected from the G0, G1, or G2 glycoform.

In an embodiment, the glycoprotein or antibody composition with mammalian-like N-glycans, preferably produced by an alg3 knock-out strain, include glycoforms that essentially lack or are devoid of glycans Manα3[Manα6(Manα3)Manα6]Manβ4GlcNAβ4GlcNAc (Man5). In specific embodiments, the filamentous fungal cell produces heterologous glycoproteins or antibodies with, as major glycoform, the trimannosyl N-glycan structure Manα3[Manα6]Manβ4GlcNAcβ4GlcNAc. In other embodiments, the filamentous fungal cell produces glycoproteins or antibodies with, as major glycoform, the G0 N-glycan structure GlcNAcβ2Manα3[GlcNAcβ2Manα6]Manβ4GlcNAcβ4GlcNAc.

In certain embodiments, the filamentous fungal cell of the invention produces glycoprotein or antibody composition with a mixture of different N-glycans.

In some embodiments, Man3GlcNAc2 N-glycan (i.e. Manα3[Manα6]Manβ4GlcNAcβ4GlcNAc) represents at least 10%, at least 20%, at least at least 30%, at least 40%, at least 50%, at least 60%, at least 70%, at least 80%, at least 90% or more of total (mol %) neutral N-glycans of the heterologous glycoprotein or antibody, as expressed in a filamentous fungal cells of the invention.

In other embodiments, GlcNAc2Man3 N-glycan (for example G0 GlcNAcβ2Manα3[GlcNAcβ2Manα6]Manβ4GlcNAcβ4GlcNAc) represents at least 10%, at least 20%, at least at least 30%, at least 40%, at least 50%, at least 60%, at least 70%, at least 80%, at least 90% or more of total (mol %) neutral N-glycans of the heterologous glycoprotein or antibody, as expressed in a filamentous fungal cells of the invention.

In other embodiments, GalGlcNAc2Man3GlcNAc2 N-glycan (for example G1 N-glycan) represents at least 10%, at least 20%, at least at least 30%, at least 40%, at least 50%, at least 60%, at least 70%, at least 80%, at least 90% or more of total (mol %) neutral N-glycans of the heterologous glycoprotein or antibody, as expressed in a filamentous fungal cells of the invention.

In other embodiments, Gal2GlcNAc2Man3GlcNAc2 N-glycan (for example G2 N-glycan) represents at least 10%, at least 20%, at least at least 30%, at least 40%, at least 50%, at least 60%, at least 70%, at least 80%, at least 90% or more of total (mol %) neutral N-glycans of the heterologous glycoprotein or antibody, as expressed in a filamentous fungal cells of the invention.

In other embodiments, complex type N-glycan represents at least 10%, at least 20%, at least at least 30%, at least 40%, at least 50%, at least 60%, at least 70%, at least 80%, at least 90% or more of total (mol %) neutral N-glycans of a heterologous glycoprotein or antibody, as expressed in a filamentous fungal cells of the invention.

In other embodiments, hybrid type N-glycan represents at least 10%, at least 20%, at least at least 30%, at least 40%, at least 50%, at least 60%, at least 70%, at least 80%, at least 90% or more of total (mol %) neutral N-glycans of a heterologous glycoprotein or antibody, as expressed in a filamentous fungal cells of the invention.

In other embodiments, less than 0.5%, 0.1%, 0.05%, or less than 0.01% of the N-glycan of the heterologous glycoprotein composition or antibody composition produced by the host cell of the invention, comprises galactose. In certain embodiments, none of N-glycans comprise galactose.

The Neu5Gc and Gala- (non-reducing end terminal Galα3Galβ4GlcNAc) structures are known xenoantigenic (animal derived) modifications of antibodies which are produced in animal cells such as CHO cells. The structures may be antigenic and, thus, harmful even at low concentrations. The filamentous fungi of the present invention lack biosynthetic pathways to produce the terminal Neu5Gc and Galα-structures. In an embodiment that may be combined with the preceding embodiments less than 0.1%, 0.01%, 0.001% or 0% of the N-glycans and/or O-glycans of the glycoprotein or antibody composition comprises Neu5Gc and/or Galα-structure. In an embodiment that may be combined with the preceding embodiments, less than 0.1%, 0.01%, 0.001% or 0% of the N-glycans and/or O-glycans of the heterologous glycoprotein or antibody composition comprises Neu5Gc and/or Galα-structure.

The filamentous fungal cells of the present invention lack genes to produce fucosylated heterologous proteins. In an embodiment that may be combined with the preceding embodiments, less than 0.1%, 0.01%, 0.001%, or 0% of the N-glycan of the glycoprotein or antibody composition comprises core fucose structures.

The terminal Galβ4GlcNAc structure of N-glycan of mammalian cell produced glycans affects bioactivity of antibodies and Galβ3GlcNAc may be xenoantigen structure from plant cell produced proteins. In an embodiment that may be combined with one or more of the preceding embodiments, less than 0.1%, 0.01%, 0.001%, or 0% of N-glycan of the heterologous glycoprotein or antibody composition comprises terminal galactose epitopes Galβ3/4GlcNAc.

Glycation is a common post-translational modification of proteins, resulting from the chemical reaction between reducing sugars such as glucose and the primary amino groups on protein. Glycation occurs typically in neutral or slightly alkaline pH in cell cultures conditions, for example, when producing antibodies in CHO cells and analysing them (see, for example, Zhang et al. (2008) Unveiling a glycation hot spot in a recombinant humanized monoclonal antibody. Anal Chem. 80(7):2379-2390). As filamentous fungi of the present invention are typically cultured in acidic pH, occurrence of glycation is reduced. In an embodiment that may be combined with the preceding embodiments, less than 1.0%, 0.5%, 0.1%, 0.01%, 0.001%, or 0% of the heterologous glycoprotein or antibody composition comprises glycation structures.

In one embodiment, the glycoprotein composition, such as an antibody is devoid of one, two, three, four, five, or six of the structures selected from the group of Neu5Gc, terminal Galα3Galβ4GlcNAc, terminal Galβ4GlcNAc, terminal Galβ3GlcNAc, core linked fucose and glycation structures.

In certain embodiments, such glycoprotein protein with mammalian-like N-glycan, as produced in the filamentous fungal cell of the invention, is a therapeutic protein. Therapeutic proteins may include immunoglobulin, or a protein fusion comprising a Fc fragment or other therapeutic glycoproteins, such as antibodies, erythropoietins, interferons, growth hormones, albumins or serum albumin, enzymes, or blood-clotting factors and may be useful in the treatment of humans or animals. For example, the glycoproteins with mammalian-like N-glycan as produced by the filamentous fungal cell according to the invention may be a therapeutic glycoprotein such as rituximab.

Methods for producing glycoproteins with mammalian-like N-glycans in filamentous fungal cells are also described for example in WO2012/069593.

In one aspect, the filamentous fungal cell according to the invention as described above, is further genetically modified to mimick the traditional pathway of mammalian cells, starting from Man5 N-glycans as acceptor substrate for GnTI, and followed sequentially by GnT1, mannosidase II and GnTII reaction steps (hereafter referred as the “traditional pathway” for producing G0 glycoforms). In one variant, a single recombinant enzyme comprising the catalytic domains of GnTI and GnTII, is used.

Alternatively, in a second aspect, the filamentous fungal cell according to the invention as described above is further genetically modified to have alg3 reduced expression, allowing the production of core Man₃GlcNAc₂ N-glycans, as acceptor substrate for GnTI and GnTII subsequent reactions and bypassing the need for mannosidase α1,2 or mannosidase II enzymes (the reduced “alg3” pathway). In one variant, a single recombinant enzyme comprising the catalytic domains of GnTI and GnTII, is used.

In such embodiments for mimicking the traditional pathway for producing glycoproteins with mammalian-like N-glycans, a Man₅ expressing filamentous fungal cell, such as T. reesei strain, may be transformed with a GnTI or a GnTII/GnTI fusion enzyme using random integration or by targeted integration to a known site known not to affect Man5 glycosylation. Strains that synthesise GlcNAcMan5 N-glycan for production of proteins having hybrid type glycan(s) are selected. The selected strains are further transformed with a catalytic domain of a mannosidase II-type mannosidase capable of cleaving Man5 structures to generate GlcNAcMan3 for production of proteins having the corresponding GlcNAcMan3 glycoform or their derivative(s). In certain embodiments, mannosidase II-type enzymes belong to glycoside hydrolase family 38 (cazy.org/GH38_all.html). Characterized enzymes include enzymes listed in cazy.org/GH38_characterized.html. Especially useful enzymes are Golgi-type enzymes that cleaving glycoproteins, such as those of subfamily α-mannosidase II (Man2A1; ManA2). Examples of such enzymes include human enzyme AAC50302, D. melanogaster enzyme (Van den Elsen J. M. et al (2001) EMBO J. 20: 3008-3017), those with the 3D structure according to PDB-reference 1HTY, and others referenced with the catalytic domain in PDB. For cytoplasmic expression, the catalytic domain of the mannosidase is typically fused with an N-terminal targeting peptide (for example as disclosed in the above Section) or expressed with endogenous animal or plant Golgi targeting structures of animal or plant mannosidase II enzymes. After transformation with the catalytic domain of a mannosidase II-type mannosidase, strains are selected that produce GlcNAcMan3 (if GnTI is expressed) or strains are selected that effectively produce GlcNAc2Man3 (if a fusion of GnTI and GnTII is expressed). For strains producing GlcNAcMan3, such strains are further transformed with a polynucleotide encoding a catalytic domain of GnTII and transformant strains that are capable of producing GlcNAc2Man3GlcNAc2 are selected.

In such embodiment for mimicking the traditional pathway, the filamentous fungal cell is a filamentous fungal cell as defined in previous sections, and further comprising one or more polynucleotides encoding a polypeptide selected from the group consisting of:
i) α1,2 mannosidase,
ii)N-acetylglucosaminyltransferase I catalytic domain,
iii) a mannosidase II,
iv)N-acetylglucosaminyltransferase II catalytic domain,
v) β1,4 galactosyltransferase, and,
vi) fucosyltransferase.

In embodiments using the reduced alg3 pathway, the filamentous fungal cell, such as a Trichoderma cell, has a reduced level of activity of a dolichyl-P-Man:Man(5)GlcNAc(2)-PP-dolichyl mannosyltransferase compared to the level of activity in a parent host cell. Dolichyl-P-Man:Man(5)GlcNAc(2)-PP-dolichyl mannosyltransferase (EC 2.4.1.130) transfers an alpha-D-mannosyl residue from dolichyl-phosphate D-mannose into a membrane lipid-linked oligosaccharide. Typically, the dolichyl-P-Man:Man(5)GlcNAc(2)-PP-dolichyl mannosyltransferase enzyme is encoded by an alg3 gene. In certain embodiments, the filamentous fungal cell for producing glycoproteins with mammalian-like N-glycans has a reduced level of expression of an alg3 gene compared to the level of expression in a parent strain.

More preferably, the filamentous fungal cell comprises a mutation of alg3. The ALG3 gene may be mutated by any means known in the art, such as point mutations or deletion of the entire alg3 gene. For example, the function of the alg3 protein is reduced or eliminated by the mutation of alg3. In certain embodiments, the alg3 gene is disrupted or deleted from the filamentous fungal cell, such as Trichoderma cell. In certain embodiments, the filamentous fungal cell is a T. reesei cell. SEQ ID NOs: 36 and 37 provide, the nucleic acid and amino acid sequences of the alg3 gene in T. reesei, respectively. In an embodiment the filamentous fungal cell is used for the production of a glycoprotein, wherein the glycan(s) comprise or consist of Manα3[Manα6]Manβ4GlcNAcβ4GlcNAc, and/or a non-reducing end elongated variant thereof.

In certain embodiments, the filamentous fungal cell has a reduced level of activity of an alpha-1,6-mannosyltransferase compared to the level of activity in a parent strain. Alpha-1,6-mannosyltransferase (EC 2.4.1.232) transfers an alpha-D-mannosyl residue from GDP-mannose into a protein-linked oligosaccharide, forming an elongation initiating alpha-(1->6)-D-mannosyl-D-mannose linkage in the Golgi apparatus. Typically, the alpha-1,6-mannosyltransferase enzyme is encoded by an och1 gene. In certain embodiments, the filamentous fungal cell has a reduced level of expression of an och1 gene compared to the level of expression in a parent filamentous fungal cell. In certain embodiments, the och1 gene is deleted from the filamentous fungal cell.

The filamentous fungal cells used in the methods of producing glycoprotein with mammalian-like N-glycans may further contain a polynucleotide encoding an N-acetylglucosaminyltransferase I catalytic domain (GnTI) that catalyzes the transfer of N-acetylglucosamine to a terminal Manα3 and a polynucleotide encoding an N-acetylglucosaminyltransferase II catalytic domain (GnTII), that catalyses N-acetylglucosamine to a terminal Manα6 residue of an acceptor glycan to produce a complex N-glycan. In one embodiment, said polynucleotides encoding GnTI and GnTII are linked so as to produce a single protein fusion comprising both catalytic domains of GnTI and GnTII.

As disclosed herein, N-acetylglucosaminyltransferase I (GlcNAc-TI; GnTI; EC 2.4.1.101) catalyzes the reaction UDP-N-acetyl-D-glucosamine+3-(alpha-D-mannosyl)-beta-D-mannosyl-R<=>UDP+3-(2-(N-acetyl-beta-D-glucosaminyl)-alpha-D-mannosyl)-beta-D-mannosyl-R, where R represents the remainder of the N-linked oligosaccharide in the glycan acceptor. An N-acetylglucosaminyltransferase I catalytic domain is any portion of an N-acetylglucosaminyltransferase I enzyme that is capable of catalyzing this reaction. GnTI enzymes are listed in the CAZy database in the glycosyltransferase family 13 (cazy.org/GT13_all). Enzymatically characterized species includes A. thaliana AAR78757.1 (U.S. Pat. No. 6,653,459), C. elegans AAD03023.1 (Chen S. et al J. Biol. Chem 1999; 274(1):288-97), D. melanogaster AAF57454.1 (Sarkar & Schachter Biol Chem. 2001 February; 382(2):209-17); C. griseus AAC52872.1 (Puthalakath H. et al J. Biol. Chem 1996 271(44):27818-22); H. sapiens AAA52563.1 (Kumar R. et al Proc Natl Acad Sci USA. 1990 December; 87(24):9948-52); M. auratus AAD04130.1 (Opat As et al Biochem J. 1998 Dec. 15; 336 (Pt 3):593-8), (including an example of deactivating mutant), Rabbit, O. cuniculus AAA31493.1 (Sarkar M et al. Proc Natl Acad Sci USA. 1991 Jan. 1; 88(1):234-8). Amino acid sequences for N-acetylglucosaminyltransferase I enzymes from various organisms are described for example in PCT/EP2011/070956. Additional examples of characterized active enzymes can be found at cazy.org/GT13_characterized. The 3D structure of the catalytic domain of rabbit GnTI was defined by X-ray crystallography in Unligil U M et al. EMBO J. 2000 Oct. 16; 19(20):5269-80. The Protein Data Bank (PDB) structures for GnTI are 1FO8, 1FO9, 1FOA, 2AM3, 2AM4, 2AM5, and 2APC. In certain embodiments, the N-acetylglucosaminyltransferase I catalytic domain is from the human N-acetylglucosaminyltransferase I enzyme (SEQ ID NO: 38) or variants thereof. In certain embodiments, the N-acetylglucosaminyltransferase I catalytic domain contains a sequence that is at least 70%, at least 75%, at least 80%, at least 85%, at least 90%, at least 95%, at least 96%, at least 97%, at least 98%, at least 99%, or 100% identical to amino acid residues 84-445 of SEQ ID NO: 38. In some embodiments, a shorter sequence can be used as a catalytic domain (e.g. amino acid residues 105-445 of the human enzyme or amino acid residues 107-447 of the rabbit enzyme; Sarkar et al. (1998) Glycoconjugate J 15:193-197). Additional sequences that can be used as the GnTI catalytic domain include amino acid residues from about amino acid 30 to 445 of the human enzyme or any C-terminal stem domain starting between amino acid residue 30 to 105 and continuing to about amino acid 445 of the human enzyme, or corresponding homologous sequence of another GnTI or a catalytically active variant or mutant thereof. The catalytic domain may include N-terminal parts of the enzyme such as all or part of the stem domain, the transmembrane domain, or the cytoplasmic domain.

As disclosed herein, N-acetylglucosaminyltransferase II (GlcNAc-T11; GnTII; EC 2.4.1.143) catalyzes the reaction UDP-N-acetyl-D-glucosamine+6-(alpha-D-mannosyl)-beta-D-mannosyl-R<=>UDP+6-(2-(N-acetyl-beta-D-glucosaminyl)-alpha-D-mannosyl)-beta-D-mannosyl-R, where R represents the remainder of the N-linked oligosaccharide in the glycan acceptor. An N-acetylglucosaminyltransferase II catalytic domain is any portion of an N-acetylglucosaminyltransferase II enzyme that is capable of catalyzing this reaction. Amino acid sequences for N-acetylglucosaminyltransferase II enzymes from various organisms are listed in WO2012069593. In certain embodiments, the N-acetylglucosaminyltransferase II catalytic domain is from the human N-acetylglucosaminyltransferase II enzyme (SEQ ID NO: 39) or variants thereof. Additional GnTII species are listed in the CAZy database in the glycosyltransferase family 16 (cazy.org/GT16_all). Enzymatically characterized species include GnTII of C. elegans, D. melanogaster, Homo sapiens (NP 002399.1), Rattus norvegicus, Sus scrofa (cazy.org/GT16_characterized). In certain embodiments, the N-acetylglucosaminyltransferase II catalytic domain contains a sequence that is at least 70%, at least 75%, at least 80%, at least 85%, at least 90%, at least 95%, at least 96%, at least 97%, at least 98%, at least 99%, or 100% identical to amino acid residues from about 30 to about 447 of SEQ ID NO: 39. The catalytic domain may include N-terminal parts of the enzyme such as all or part of the stem domain, the transmembrane domain, or the cytoplasmic domain.

In embodiments where the filamentous fungal cell contains a fusion protein of the invention, the fusion protein may further contain a spacer in between the N-acetylglucosaminyltransferase I catalytic domain and the N-acetylglucosaminyltransferase II catalytic domain. In certain embodiments, the spacer is an EGIV spacer, a 2×G4S spacer, a 3×G4S spacer, or a CBI-II spacer. In other embodiments, the spacer contains a sequence from a stem domain.

For ER/Golgi expression the N-acetylglucosaminyltransferase I and/or N-acetylglucosaminyltransferase II catalytic domain is typically fused with a targeting peptide or a part of an ER or early Golgi protein, or expressed with an endogenous ER targeting structures of an animal or plant N-acetylglucosaminyltransferase enzyme. In certain preferred embodiments, the N-acetylglucosaminyltransferase I and/or N-acetylglucosaminyltransferase II catalytic domain contains any of the targeting peptides of the invention as described in the section entitled “Targeting sequences”. Preferably, the targeting peptide is linked to the N-terminal end of the catalytic domain. In some embodiments, the targeting peptide contains any of the stem domains of the invention as described in the section entitled “Targeting sequences”. In certain preferred embodiments, the targeting peptide is a Kre2/Mnt1 targeting peptide. In other embodiments, the targeting peptide further contains a transmembrane domain linked to the N-terminal end of the stem domain or a cytoplasmic domain linked to the N-terminal end of the stem domain. In embodiments where the targeting peptide further contains a transmembrane domain, the targeting peptide may further contain a cytoplasmic domain linked to the N-terminal end of the transmembrane domain.

The filamentous fungal cells may also contain a polynucleotide encoding a UDP-GlcNAc transporter. The polynucleotide encoding the UDP-GlcNAc transporter may be endogenous (i.e., naturally present) in the host cell, or it may be heterologous to the filamentous fungal cell.

In certain embodiments, the filamentous fungal cell may further contain a polynucleotide encoding a α-1,2-mannosidase. The polynucleotide encoding the α-1,2-mannosidase may be endogenous in the host cell, or it may be heterologous to the host cell. Heterologous polynucleotides are especially useful for a host cell expressing high-mannose glycans transferred from the Golgi to the ER without effective exo-α-2-mannosidase cleavage. The α-1,2-mannosidase may be a mannosidase I type enzyme belonging to the glycoside hydrolase family 47 (cazy.org/GH47_all.html). In certain embodiments the α-1,2-mannosidase is an enzyme listed at cazy.org/GH47_characterized.html. In particular, the α-1,2-mannosidase may be an ER-type enzyme that cleaves glycoproteins such as enzymes in the subfamily of ER α-mannosidase I EC 3.2.1.113 enzymes. Examples of such enzymes include human α-2-mannosidase 1B (AAC26169), a combination of mammalian ER mannosidases, or a filamentous fungal enzyme such as α-1,2-mannosidase (MDS1) (T. reesei AAF34579; Maras M et al J Biotech. 77, 2000, 255, or Trire 45717). For ER expression, the catalytic domain of the mannosidase is typically fused with a targeting peptide, such as HDEL, KDEL, or part of an ER or early Golgi protein, or expressed with an endogenous ER targeting structures of an animal or plant mannosidase I enzyme.

In certain embodiments, the filamentous fungal cell may also further contain a polynucleotide encoding a galactosyltransferase. Galactosyltransferases transfer 3-linked galactosyl residues to terminal N-acetylglucosaminyl residue. In certain embodiments the galactosyltransferase is a 3-1,4-galactosyltransferase. Generally, 3-1,4-galactosyltransferases belong to the CAZy glycosyltransferase family 7 (cazy.org/GT7_all.html) and include β-N-acetylglucosaminyl-glycopeptide β-1,4-galactosyltransferase (EC 2.4.1.38), which is also known as N-acetylactosamine synthase (EC 2.4.1.90). Useful subfamilies include β4-GalT1, β4-GalT-II, -III, -IV, -V, and -VI, such as mammalian or human 1β4-GalTI or β4GalT-II, -III, -IV, -V, and -VI or any combinations thereof. β4-GalT1, β4-GalTII, or β4-GalTIII are especially useful for galactosylation of terminal GlcNAcβ2-structures on N-glycans such as GlcNAcMan3, GlcNAc2Man3, or GlcNAcMan5 (Guo S. et al. Glycobiology 2001, 11:813-20). The three-dimensional structure of the catalytic region is known (e.g. (2006) J. Mol. Biol. 357: 1619-1633), and the structure has been represented in the PDB database with code 2FYD. The CAZy database includes examples of certain enzymes. Characterized enzymes are also listed in the CAZy database at cazy.org/GT7_characterized.html. Examples of useful β4GalT enzymes include β4GalT1, e.g. bovine Bos taurus enzyme AAA30534.1 (Shaper N. L. et al Proc. Natl. Acad. Sci. U.S.A. 83 (6), 1573-1577 (1986)), human enzyme (Guo S. et al. Glycobiology 2001, 11:813-20), and Mus musculus enzyme AAA37297 (Shaper, N. L. et al. 1998 J. Biol. Chem. 263 (21), 10420-10428); β4GalTII enzymes such as human β4GalTII BAA75819.1, Chinese hamster Cricetulus griseus AAM77195, Mus musculus enzyme BAA34385, and Japanese Medaka fish Oryzias latipes BAH36754; and β4GalTIII enzymes such as human β4GalTIII BAA75820.1, Chinese hamster Cricetulus griseus AAM77196 and Mus musculus enzyme AAF22221.

The galactosyltransferase may be expressed in the plasma membrane of the host cell. A heterologous targeting peptide, such as a Kre2 peptide described in Schwientek J. Biol. Chem 1996 3398, may be used. Promoters that may be used for expression of the galactosyltransferase include constitutive promoters such as gpd, promoters of endogenous glycosylation enzymes and glycosyltransferases such as mannosyltransferases that synthesize N-glycans in the Golgi or ER, and inducible promoters of high-yield endogenous proteins such as the cbh1 promoter.

In certain embodiments of the invention where the filamentous fungal cell contains a polynucleotide encoding a galactosyltransferase, the filamentous fungal cell also contains a polynucleotide encoding a UDP-Gal 4 epimerase and/or UDP-Gal transporter. In certain embodiments of the invention where the filamentous fungal cell contains a polynucleotide encoding a galactosyltransferase, lactose may be used as the carbon source instead of glucose when culturing the host cell. The culture medium may be between pH 4.5 and 7.0 or between 5.0 and 6.5. In certain embodiments of the invention where the filamentous fungal cell contains a polynucleotide encoding a galactosyltransferase and a polynucleotide encoding a UDP-Gal 4 epimerase and/or UDP-Gal transporter, a divalent cation such as Mn2+, Ca2+ or Mg2+ may be added to the cell culture medium.

Accordingly, in certain embodiments, the filamentous fungal cell of the invention, for example, selected among Neurospora, Trichoderma, Myceliophthora, Aspergillus, Fusarium or Chrysosporium cell, and more preferably Trichoderma reesei cell, may comprise the following features:

a) a mutation in at least one endogenous protease that reduces or eliminates the activity of said endogenous protease, preferably the protease activity of two or three or more endogenous proteases is reduced, for example, pep1, tsp1, gap1 and/or slp1 proteases, in order to improve production or stability of a heterologous glycoprotein to be produced,

b) a polynucleotide encoding a heterologous catalytic subunit of oligosaccharyl transferase, preferably of SEQ ID NO:2 or NO:9,

c) a polynucleotide encoding a glycoprotein having at least one asparagine, preferably a heterologous glycoprotein, such as an immunoglobulin, an antibody, or a protein fusion comprising Fc fragment of an immunoglobulin.

d) optionally, a deletion or disruption of the alg3 gene,

e) optionally, a polynucleotide encoding N-acetylglucosaminyltransferase I catalytic domain and a polynucleotide encoding N-acetylglucosaminyltransferase II catalytic domain,

f) optionally, a polynucleotide encoding β1,4 galactosyltransferase,

g) optionally, a polynucleotide or polynucleotides encoding UDP-Gal 4 epimerase and/or transporter.

Targeting Sequences

In certain embodiments, recombinant enzymes, such as α1,2 mannosidases, GnTI, or other glycosyltransferases introduced into the filamentous fungal cells, include a targeting peptide linked to the catalytic domains. The term “linked” as used herein means that two polymers of amino acid residues in the case of a polypeptide or two polymers of nucleotides in the case of a polynucleotide are either coupled directly adjacent to each other or are within the same polypeptide or polynucleotide but are separated by intervening amino acid residues or nucleotides. A “targeting peptide”, as used herein, refers to any number of consecutive amino acid residues of the recombinant protein that are capable of localizing the recombinant protein to the endoplasmic reticulum (ER) or Golgi apparatus (Golgi) within the host cell. The targeting peptide may be N-terminal or C-terminal to the catalytic domains. In certain embodiments, the targeting peptide is N-terminal to the catalytic domains. In certain embodiments, the targeting peptide provides binding to an ER or Golgi component, such as to a mannosidase II enzyme. In other embodiments, the targeting peptide provides direct binding to the ER or Golgi membrane.

Components of the targeting peptide may come from any enzyme that normally resides in the ER or Golgi apparatus. Such enzymes include mannosidases, mannosyltransferases, glycosyltransferases, Type 2 Golgi proteins, and MNN2, MNN4, MNN6, MNN9, MNN10, MNS1, KRE2, VAN1, and OCH1 enzymes. Such enzymes may come from a yeast or fungal species such as those of Acremonium, Aspergillus, Aureobasidium, Cryptococcus, Chrysosporium, Chrysosporium lucknowense, Filobasidium, Fusarium, Gibberella, Humicola, Magnaporthe, Mucor, Myceliophthora, Myrothecium, Neocallimastix, Neurospora, Paecilomyces, Penicillium, Piromyces, Schizophyllum, Talaromyces, Thermoascus, Thielavia, Tolypocladium, and Trichoderma. Sequences for such enzymes can be found in the Gen Bank sequence database.

In certain embodiments the targeting peptide comes from the same enzyme and organism as one of the catalytic domains of the recombinant protein. For example, if the recombinant protein includes a human GnTII catalytic domain, the targeting peptide of the recombinant protein is from the human GnTII enzyme. In other embodiments, the targeting peptide may come from a different enzyme and/or organism as the catalytic domains of the recombinant protein.

Examples of various targeting peptides for use in targeting proteins to the ER or Golgi that may be used for targeting the recombinant enzymes, include: Kre2/Mnt1 N-terminal peptide fused to galactosyltransferase (Schwientek, J B C 1996, 3398), HDEL for localization of mannosidase to ER of yeast cells to produce Man5 (Chiba, J B C 1998, 26298-304; Callewaert, FEBS Lett 2001, 173-178), OCH1 targeting peptide fused to GnTI catalytic domain (Yoshida et al, Glycobiology 1999, 53-8), yeast N-terminal peptide of Mns1 fused to α2-mannosidase (Martinet et al, Biotech Lett 1998, 1171), N-terminal portion of Kre2 linked to catalytic domain of GnTI or β4GalT (Vervecken, Appl. Environ Microb 2004, 2639-46), various approaches reviewed in Wildt and Gerngross (Nature Rev Biotech 2005, 119), full-length GnTI in Aspergillus nidulans (Kalsner et al, Glycocon. J 1995, 360-370), full-length GnTI in Aspergillus oryzae (Kasajima et al, Biosci Biotech Biochem 2006, 2662-8), portion of yeast Sec12 localization structure fused to C. elegans GnTI in Aspergillus (Kainz et al 2008), N-terminal portion of yeast Mnn9 fused to human GnTI in Aspergillus (Kainz et al 2008), N-terminal portion of Aspergillus Mnn10 fused to human GnTI (Kainz et al, Appl. Environ Microb 2008, 1076-86), and full-length human GnTI in T. reesei (Maras et al, FEBS Lett 1999, 365-70).

In certain embodiments the targeting peptide is an N-terminal portion of the Mnt1/Kre2 targeting peptide having the amino acid sequence of SEQ ID NO: 40 (for example encoded by the polynucleotide of SEQ ID NO:41). In certain embodiments, the targeting peptide is selected from human GNT2, KRE2, KRE2-like, Och1, Anp1, Van1 as shown in the Table 1 below:

TABLE 1
Amino acid sequence of targeting peptides

| Protein | TreID | Amino acid sequence |
|---|---|---|
| human GNT2 | — | MRFRIYKRKVLILTLVVAACGFVLWSSNGRQR |
| | | KNEALAPPLLDAEPARGAGGRGGDHP |
| | | (SEQ ID NO: 42) |
| KRE2 | 21576 | MASTNARYVRYLLIAFFTILVFYFVSNSKYEGV |
| | | DLNKGTFTAPDSTKTTPK |
| | | (SEQ ID NO: 43) |
| KRE2-like | 69211 | MAIARPVRALGGLAAILWCFFLYQLLRPSSSY |
| | | NSPGDRYINFERDPNLDPTG |
| | | (SEQ ID NO: 44) |
| Och1 | 65646 | MLNPRRALIAAAFILTVFFLISRSHNSESASTS |
| | | (SEQ ID NO: 45) |
| Anp1 | 82551 | MMPRHHSSGFSNGYPRADTFEISPHRFQPRA |
| | | TLPPHRKRKRTAIRVGIAVVVILVLVLWFGQPR |
| | | SVASLISLGILSGYDDLKLE |
| | | (SEQ ID NO: 46) |
| Van1 | 81211 | MLLPKGGLDWRSARAQIPPTRALWNAVTRTR |
| | | FILLVGITGLILLLWRGVSTSASE |
| | | (SEQ ID NO: 47) |

Further examples of sequences that may be used for targeting peptides include the targeting sequences as described in WO2012/069593.

Uncharacterized sequences may be tested for use as targeting peptides by expressing enzymes of the glycosylation pathway in a host cell, where one of the enzymes contains the uncharacterized sequence as the sole targeting peptide, and measuring the glycans produced in view of the cytoplasmic localization of glycan biosynthesis (e.g. as in Schwientek J B C 1996 3398), or by expressing a fluorescent reporter protein fused with the targeting peptide, and analysing the localization of the protein in the Golgi by immunofluorescence or by fractionating the cytoplasmic membranes of the Golgi and measuring the location of the protein.

Methods for producing a glycoprotein having increased N-glycosylation site occupancy

The filamentous fungal cells as described above are useful in methods for producing a glycoprotein composition with increased N-glycosylation site occupancy.

Accordingly, in another aspect, the invention relates to a method for producing a glycoprotein composition with increased N-glycosylation site occupancy, comprising

a) providing a filamentous fungal cell, for example a Trichoderma cell, having a Leishmania STT3D gene encoding a catalytic subunit of oligosaccharyl transferase, or a functional variant thereof, and a polynucleotide encoding a heterologous glycoprotein,

b) culturing the cell under appropriate conditions for expression of the STT3D gene or its functional variant, and the production of the heterologous glycoprotein; and,

c) recovering said glycoprotein composition and, optionally, purifying the heterologous glycoprotein composition.

In specific embodiments of the method, the filamentous fungal cell comprises one or more mutation that reduces or eliminates one or more endogenous protease activity compared to a parental filamentous fungal cell which does not have said mutation(s), as described above.

In methods of the invention, certain growth media include, for example, common commercially-prepared media such as Luria-Bertani (LB) broth, Sabouraud Dextrose (SD) broth or Yeast medium (YM) broth. Other defined or synthetic growth media may also be used and the appropriate medium for growth of the particular host cell will be known by someone skilled in the art of microbiology or fermentation science. Culture medium typically has the Trichoderma reesei minimal medium (Pentla et al., 1987, Gene 61, 155-164) as a basis, supplemented with substances inducing the production promoter such as lactose, cellulose, spent grain or sophorose. Temperature ranges and other conditions suitable for growth are known in the art (see, e.g., Bailey and Ollis 1986). In certain embodiments the pH of cell culture is between 3.5 and 7.5, between 4.0 and 7.0, between 4.5 and 6.5, between 5 and 5.5, or at 5.5. In certain embodiments, to produce an antibody the filamentous fungal cell or Trichoderma fungal cell is cultured at a pH range selected from 4.7 to 6.5; pH 4.8 to 6.0; pH 4.9 to 5.9; and pH 5.0 to 5.8.

In some embodiments of the invention, the method comprises culturing in a medium comprising one or two protease inhibitors.

In a specific embodiment of the invention, the method comprises culturing in a medium comprising one or two protease inhibitors selected from SBTI and chymostatin.

In some embodiments, the glycoprotein is a heterologous glycoprotein, preferably a mammalian glycoprotein. In other embodiments, the heterologous glycoprotein is a non-mammalian glycoprotein.

In certain embodiments, a mammalian glycoprotein is selected from an immunoglobulin, immunoglobulin or antibody heavy or light chain, a monoclonal antibody, a Fab fragment, an F(ab′)2 antibody fragment, a single chain antibody, a monomeric or multimeric single domain antibody, a camelid antibody, or their antigen-binding fragments.

A fragment of a protein, as used herein, consists of at least 10, 20, 30, 40, 50, 60, 70, 80, 90, 100 consecutive amino acids of a reference protein.

As used herein, an “immunoglobulin” refers to a multimeric protein containing a heavy chain and a light chain covalently coupled together and capable of specifically combining with antigen. Immunoglobulin molecules are a large family of molecules that include several types of molecules such as IgM, IgD, IgG, IgA, and IgE.

As used herein, an “antibody” refers to intact immunoglobulin molecules, as well as fragments thereof which are capable of binding an antigen. These include hybrid (chimeric) antibody molecules (see, e.g., Winter et al. Nature 349:293-99225, 1991; and U.S. Pat. No. 4,816,567 226); F(ab′)2 molecules; non-covalent heterodimers; dimeric and trimeric antibody fragment constructs; humanized antibody molecules (see e.g., Riechmann et al. Nature 332, 323-27, 1988; Verhoeyan et al. Science 239, 1534-36, 1988; and GB 2,276,169); and any functional fragments obtained from such molecules, as well as antibodies obtained through non-conventional processes such as phage display or transgenic mice. Preferably, the antibodies are classical antibodies with Fc region. Methods of manufacturing antibodies are well known in the art.

In further embodiments, the yield of the mammalian glycoprotein, for example, the antibody, is at least 0.5, at least 1, at least 2, at least 3, at least 4, or at least 5 grams per liter.

In certain embodiments, the mammalian glycoprotein is an antibody, optionally, IgG1, IgG2, IgG3, or IgG4. In further embodiments, the yield of the antibody is at least 0.5, at least 1, at least 2, at least 3, at least 4, or at least 5 grams per liter. In further embodiments, the mammalian glycoprotein is an antibody, and the antibody contains at least 70%, at least 80%, at least 90%, at least 95%, or at least 98% of a natural antibody C-terminus and N-terminus without additional amino acid residues. In other embodiments, the mammalian glycoprotein is an antibody, and the antibody contains at least 70%, at least 80%, at least 90%, at least 95%, or at least 98% of a natural antibody C-terminus and N-terminus that do not lack any C-terminal or N-terminal amino acid residues.

In certain embodiments where the mammalian glycoprotein (e.g. the antibody) is purified from cell culture, the culture containing the mammalian glycoprotein contains polypeptide fragments that make up a mass percentage that is less than 50%, less than 40%, less than 30%, less than 20%, or less than 10% of the mass of the produced polypeptides. In certain preferred embodiments, the mammalian glycoprotein is an antibody, and the polypeptide fragments are heavy chain fragments and/or light chain fragments. In other embodiments, where the mammalian glycoprotein is an antibody and the antibody purified from cell culture, the culture containing the antibody contains free heavy chains and/or free light chains that make up a mass percentage that is less than 50%, less than 40%, less than 30%, less than 20%, or less than 10% of the mass of the produced antibody. Methods of determining the mass percentage of polypeptide fragments are well known in the art and include, measuring signal intensity from an SDS-gel.

In other embodiments, the heterologous glycoprotein (e.g. the antibody) with increased N-glycosylation site occupancy, for example, the antibody, comprises the trimannosyl N-glycan structure Manα3[Manα6]Manβ4GlcNAcβ4GlcNAc. In some embodiments, the Manα3[Manα6]Manβ4GlcNAcβ4GlcNAc structure represents at least 20%, 30%; 40%, 50%; 60%, 70%, 80% (mol %) or more, of the total N-glycans of the heterologous glycoprotein (e.g. the antibody) composition obtained by the methods of the invention. In other embodiments, the heterologous glycoprotein (e.g. the antibody) comprises the G0 N-glycan structure GlcNAcβ2Manα3[GlcNAcβ2Manα6]Manβ4GlcNAcβ4GlcNAc. In other embodiments, the non-fucosylated G0 glycoform structure represents at least 20%, 30%; 40%, 50%; 60%, 70%, 80% (mol %) or more, of the total N-glycans of the heterologous glycoprotein (e.g. the antibody) composition obtained by the methods of the invention. In other embodiments, galactosylated N-glycans represents less (mol %) than 0.5%, 0.1%, 0.05%, 0.01% of total N-glycans of the culture, and/or of the heterologous glycoprotein with increased N-glycosylation site occupancy. In certain embodiments, the culture or the heterologous glycoprotein, for example an antibody, comprises no galactosylated N-glycans.

In certain embodiments of any of the disclosed methods, the method includes the further step of providing one or more, two or more, three or more, four or more, or five or more protease inhibitors. In certain embodiments, the protease inhibitors are peptides that are co-expressed with the mammalian glycoprotein. In other embodiments, the inhibitors inhibit at least two, at least three, or at least four proteases from a protease family selected from aspartic proteases, trypsin-like serine proteases, subtilisin proteases, and glutamic proteases.

In certain embodiments of any of the disclosed methods, the filamentous fungal cell or Trichoderma fungal cell also contains a carrier protein. As used herein, a “carrier protein” is portion of a protein that is endogenous to and highly secreted by a filamentous fungal cell or Trichoderma fungal cell. Suitable carrier proteins include, without limitation, those of T. reesei mannanase I (Man5A, or MANI), T. reesei cellobiohydrolase II (Cel6A, or CBHII) (see, e.g., Paloheimo et al Appl. Environ. Microbiol. 2003 December; 69(12): 7073-7082) or T. reesei cellobiohydrolase I (CBHI). In some embodiments, the carrier protein is CBH1. In other embodiments, the carrier protein is a truncated T. reesei CBH1 protein that includes the CBH1 core region and part of the CBH1 linker region. In some embodiments, a carrier such as a cellobiohydrolase or its fragment is fused to an antibody light chain and/or an antibody heavy chain. In some embodiments, a carrier-antibody fusion polypeptide comprises a Kex2 cleavage site. In certain embodiments, Kex2, or other carrier cleaving enzyme, is endogenous to a filamentous fungal cell. In certain embodiments, carrier cleaving protease is heterologous to the filamentous fungal cell, for example, another Kex2 protein derived from yeast or a TEV protease. In certain embodiments, carrier cleaving enzyme is overexpressed. In certain embodiments, the carrier consists of about 469 to 478 amino acids of N-terminal part of the T. reesei CBH1 protein GenBank accession No. EGR44817.1.

In one embodiment, the polynucleotide encoding the heterologous glycoprotein (e.g. the antibody) further comprises a polynucleotide encoding CBH1 catalytic domain and linker as a carrier protein, and/or cbh1 promoter.

In certain embodiments, the filamentous fungal cell of the invention overexpress KEX2 protease. In an embodiment the heterologous glycoprotein (e.g. the antibody) is expressed as fusion construct comprising an endogenous fungal polypeptide, a protease site such as a Kex2 cleavage site, and the heterologous protein such as an antibody heavy and/or light chain. Useful 2-7 amino acids combinations preceding Kex2 cleavage site have been described, for example, in Mikosch et al.

(1996) J. Biotechnol. 52:97-106; Goller et al. (1998) Appl Environ Microbiol. 64:3202-3208; Spencer et al. (1998) Eur. J. Biochem. 258:107-112; Jalving et al. (2000) Appl. Environ. Microbiol. 66:363-368; Ward et al. (2004) Appl. Environ. Microbiol. 70:2567-2576; Ahn et al. (2004) Appl. Microbiol. Biotechnol. 64:833-839; Paloheimo et al. (2007) Appl Environ Microbiol. 73:3215-3224; Paloheimo et al. (2003) Appl Environ Microbiol. 69:7073-7082; and Margolles-Clark et al. (1996) Eur J Biochem. 237:553-560.

The invention further relates to the glycoprotein composition, for example the antibody composition, obtainable or obtained by the method as disclosed above.

In other specific embodiments, such glycoprotein or antibody composition further comprises as 50%, 60%, 70% or 80% (mole % neutral N-glycan), of the following glycoform:
(i) Manα3[Manα6(Manα3)Manα6]Manβ4GlcNAβ4GlcNAc (Man5 glycoform);
(ii) GlcNAcβ2Manα3[Manα6(Manα3)Manα6]Manβ4GlcNAβ4GlcNAc, or β4-galactosylated variant thereof;
(iii) Manα6(Manα3)Manβ4GlcNAβ4GlcNAc;
(iv) Manα6(GlcNAcβ2Manα3)Manβ4GlcNAβ4GlcNAc, or (4-galactosylated variant thereof: or,
(v) complex type N-glycans selected from the G0, G1 or G2 glycoform.

In some embodiments the N-glycan glycoform according to iii-v comprises less than 15%, 10%, 7%, 5%, 3%, 1% or 0.5% or is devoid of Man5 glycan as defined in i) above.

### EXAMPLES

Functional Assays

Assay for Measuring Total Protease Activity of Cells of the Invention

The protein concentrations were determined from supernatant samples from day 2-7 of 1×-7×protease deficient strains (described in PCT/EP2013/050126) according to EnzChek protease assay kit (Molecular probes #E6638, green fluorescent casein substrate). Briefly, the supernatants were diluted in sodium citrate buffer to equal total protein concentration and equal amounts of the diluted supernatants were added into a black 96 well plate, using 3 replicate wells per sample. Casein FL diluted stock made in sodium citrate buffer was added to each supernatant containing well and the plates were incubated covered in plastic bag at 37° C. The fluorescence from the wells was measured after 2, 3, and 4 hours. The readings were done on the Varioskan fluorescent plate reader using 485 nm excitation and 530 nm emission. Some protease activity measurements were performed using succinylated casein (QuantiCleave protease assay kit, Pierce #23263) according to the manufacturer's protocol.

The pep1 single deletion reduced the protease activity by 1.7-fold, the pep1/tsp1 double deletion reduced the protease activity by 2-fold, the pep1/tsp1/slp1 triple deletion reduced the protease activity by 3.2-fold, the pep1/tsp1/slp1/gap1 quadruple deletion reduced the protease activity by 7.8-fold compared to the wild type M124 strain, the pep1/tsp1/slp1/gap1/gap2 5-fold deletion reduced the protease activity by 10-fold, the pep1/tsp1/slp1/gap1/gap2/pep4 6-fold deletion reduced the protease activity by 15.9-fold, and the pep1/tsp1/slp1/gap1/gap2/pep4/pep3 7-fold deletion reduced the protease activity by 18.2-fold.

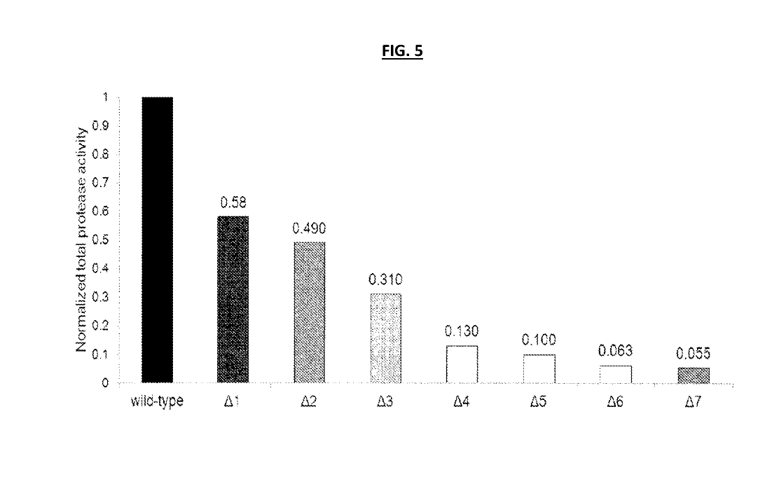

FIG. 5 graphically depicts normalized protease activity data from culture supernatants from each of the protease deletion supernatants (from 1-fold to 7-fold deletion mutant) and the parent strain without protease deletions. Protease activity was measured at pH 5.5 in first 5 strains and at pH 4.5 in the last three deletion strains. Protease activity is against green fluorescent casein. The six-fold protease deletion strain has only 6% of the wild type parent strain and the 7-fold protease deletion strain protease activity was about 40% less than the 6-fold protease deletion strain activity.

Assay for Measuring N-Glycosylation Site Occupancy in a Glycoprotein Composition 10-30 μg of antibody is digested with 13.4-30 U of FabRICATOR (Genovis), +37° C., 60 min—overnight, producing one F(ab′)2 fragment and one Fc fragment per an antibody molecule. Digested samples are purified using Poros R1 filter plate (Glyken corp.) and the Fc fragments are analysed for N-glycan site occupancy using MALDI-TOF MS. The percentage of site occupancy of an Fc is the average of two values: the one obtained from intensity values of the peaks (single and double charged) and the other from area of the peaks (single and double charged); both the values are calculated as glycosylated signal divided by the sum of non-glycosylated and glycosylated signals.

### Example 1—Generation of T. reesei Expressing L. major STT3

The Leishmania major oligosaccharyl transferase 4D (old GenBank No. XP 843223.1, new XP 003722509.1; SEQ ID NO: 1) coding sequence was codon optimized for Trichoderma reesei expression (codon optimized nucleic acid sequence SEQ ID NO: 2). The optimized coding sequence was synthesized along with cDNA1 promoter (SEQ ID NO: 3) and TrpC terminator flanking sequence (SEQ ID NO: 4). The Leishmania major STT3 gene was excised from the optimized cloning vector using PacI restriction enzyme digestion. The expression entry vector was also digested with PacI and dephosphorylated with calf alkaline phosphatase. The STT3 gene and the digested vector were separated with agarose gel electrophoresis and correct fragments were isolated from the gel with a gel extraction kit (Qiagen) according to manufacturer's protocol. The purified Leishmania major STT3 gene was ligated into the expression vector with T4 DNA ligase. The ligation reaction was transformed into chemically competent DH5a E. coli and grown on ampicillin (100 μg/ml) selection plates. Miniprep plasmid preparations were made from several colonies. The presence of the Leishmania major STT3 gene insert was checked by digesting the prepared plasmids with PacI digestion and several positive clones were sequenced to verify the gene orientation. One correctly orientated clone was chosen to be the final vector pTTv201.

The expression cassette contained the constitutive cDNA1 promoter from Trichoderma reesei to drive expression of Leishmania major STT3. The terminator sequence included in the cassette was the TrpC terminator from Aspergillus niger. The expression cassette was targeted into the xylanase 1 locus (xyn1, tre74223) using the xylanase 1 sequence from the 5′ and 3′ flanks of the gene (SEQ ID NO: 5 and SEQ ID NO: 6). These sequences were included in the cassette to allow the cassette to integrate into the xyn1 locus via homologous recombination. The cassette contained a pyr4 loopout marker for selection. The pyr4 gene encodes the orotidine-5′-monophosphate (OMP) decarboxylase of T. reesei (Smith, J. L., et al., 1991, Current Genetics 19:27-33) and is needed for uridine synthesis. Strains deficient for OMP decarboxylase activity are unable to grow on minimal medium without uridine supplementation (i.e. are uridine auxotrophs).

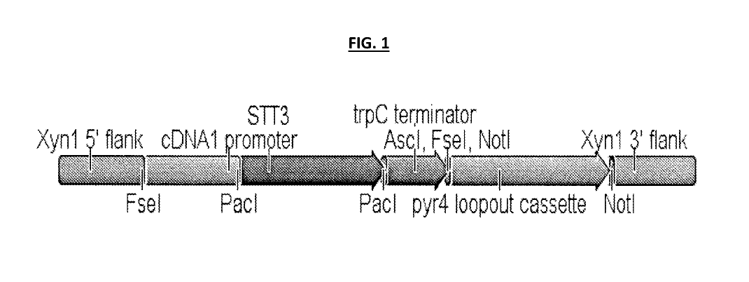

To prepare the vector for transformation, the vector was cut with PmeI to release the expression cassette (FIG. 1). The digest was separated with agarose gel electrophoresis and the correct fragment was isolated from the gel with a gel extraction kit (Qiagen) according to manufacturer's protocol. The purified expression cassette DNA (5 μg) was then transformed into protoplasts of the Trichoderma reesei strain M317 (M317 has been described in the International Patent Application No. PCT/EP2013/050126; M317 is pyr4- of M304 and it comprises MAB01 light chain fused to T. reesei truncated CBH1 carrier with NVISKR Kex2 cleavage sequence, MAB01 heavy chain fused to T. reesei truncated CBH1 carrier with AXE1 [DGETVVKR] Kex2 cleavage sequence, Δpep1Δtsp1Δslp1, and overexpression of T. reesei KEX2). Preparation of protoplasts and transformation were carried out according to methods in Penttila et al. (1987, Gene 61:155-164) and Gruber et al (1990, Curr. Genet. 18:71-76) for pyr4 selection. The transformed protoplasts were plated onto Trichoderma minimal media (TrMM) plates.

Transformants were then streaked onto TrMM plates with 0.1% TritonX-100. Transformants growing fast as selective streaks were screened by PCR using the primers listed in Table 1. DNA from mycelia was purified and analyzed by PCR to look at the integration of the 5′ and 3′ flanks of cassette and the existence of the xylanase 1 ORF. The cassette was targeted into the xylanase 1 locus; therefore the open reading frame was not present in the positively integrated transformants. To screen for 5′ integration, sequence outside of the 5′ integration flank was used to create a forward primer that would amplify genomic DNA flanking xyn1 and the reverse primer was made from sequence in the cDNA promoter of the cassette. To check for proper integration of the cassette in the 3′ flank, a forward primer was made from sequence outside of the 3′ integration flank that would amplify genomic DNA flanking xyn1 and the reverse primer was made from sequence in the pyr4 marker. Thus, one primer would amply sequence from genomic DNA outside of the cassette and the other would amply sequence from DNA in the cassette. The primer sequences are listed in Table 1. Four final strains showing proper integration and a deletion of xyn1 orf were called M420-M423.

Shake flask cultures were conducted for four of the STT3 producing strains (M420-M423) to evaluate growth characteristics and to provide samples for glycosylation site occupancy analysis. The shake flask cultures were done in TrMM, 40 g/I lactose, 20 g/I spent grain extract, 9 g/I casamino acids, 100 mM PIPPS, pH 5.5. L. major STT3 expression did not affect growth negatively when compared to the parental strain M304 (Tables 2 and 3). The cell dry weight for the STT3 expressing transformants appeared to be slightly higher compared to the parent strain M304.

TABLE 1
List of primers used for PCR screening of STT3
transformants.

| 5′ flank screening primers: | 1205 bp product |
|---|---|
| T403_Xyn1_5′flank_fwd | CCGCGTTGAACGGCTTCCCA |
| | (SEQ ID NO: 48) |
| T140_cDNA1promoter_rev | TAACTTGTACGCTCTCAGTT |
| | CGAG (SEQ ID NO: 49) |
| 3′ flank screening primers: | 1697 bp product |
| T404_Xyn1_3′flank_fwd | GCGACGGCGACCCATTAGCA |
| | (SEQ ID NO: 50) |
| T028_Pyr4_flank_rev | CATCCTCAAGGCCTCAGAC |
| | (SEQ ID NO: 51) |
| xylanase 1 orf primers: | 589 bp product |
| T405_Xyn1_orf_screen_fwd | TGCGCTCTCACCAGCATCGC |
| | (SEQ ID NO: 52) |
| T406_Xyn1_orf_screen_rev | GTCCTGGGCGAGTTCCGCAC |
| | (SEQ ID NO: 53) |

TABLE 2
Cell dry weight from large
shake flask cultures.

| Cell dry | | | |
|---|---|---|---|
| weight | | | |
| (g/L) | day 3 | day 5 | day 7 |
| M304 | 2.3 | 3.3 | 4.3 |
| M420 | 3.7 | 4.3 | 5.4 |
| M421 | 3.7 | 4.6 | 6.3 |
| M422 | 3.8 | 4.5 | 5.4 |
| M423 | 3.7 | 4.6 | 5.7 |

TABLE 3
pH values from large
shake flask cultures.

| pH | | | |
|---|---|---|---|
| values | day 3 | day 5 | day 7 |
| M304 | 5.6 | 6.1 | 6.2 |
| M420 | 6.1 | 6.1 | 6.1 |
| M421 | 6.0 | 5.9 | 6.0 |
| M422 | 6.1 | 6.1 | 6.2 |
| M423 | 6.1 | 6.1 | 6.1 |

Site Occupancy Analysis

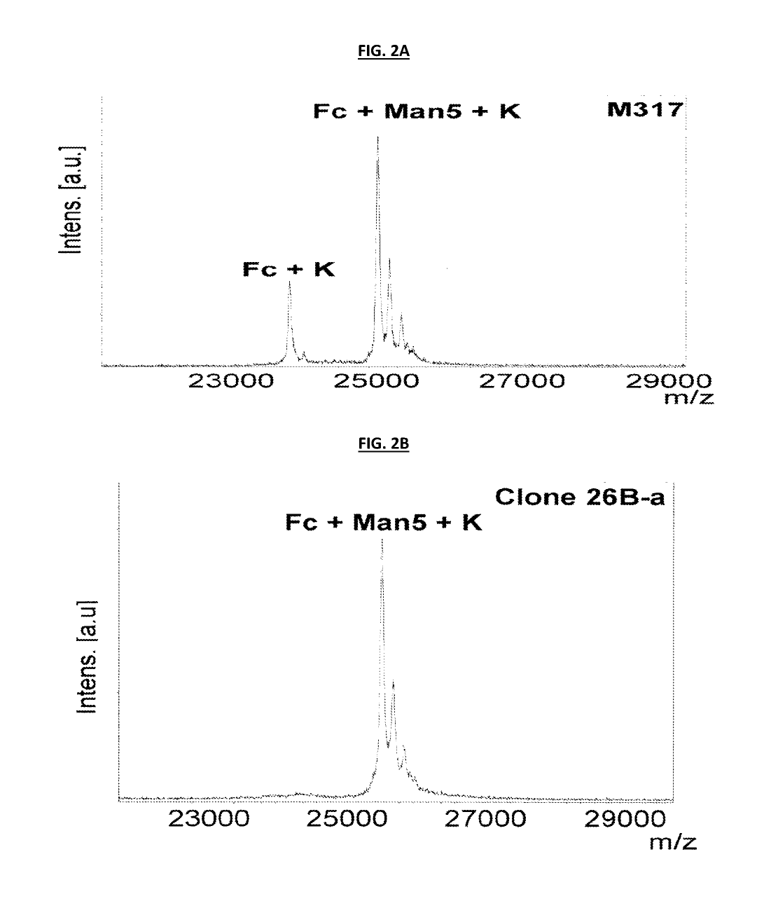

Four transformants [pTTv201; 17A-a (M420), 26B-a (M421), 65B-a (M422) and 97A-a (M423)] and their parental strain (M317) were cultivated in shake flasks and samples at day 5 and 7 time points were collected. MAB01 antibody was purified from culture supernatants using Protein G HP MultiTrap 96-well plate (GE Healthcare) according to manufacturer's instructions. The antibody was eluted with 0.1 M citrate buffer, pH 2.6 and neutralized with 2 M Tris, pH 9. The concentration was determined via UV absorbance in spectrophotometer against MAB01 standard curve. 10 μg of antibody was digested with 13.4 U of FabRICATOR (Genovis), +37° C., 60 min, producing one F(ab′)2 fragment and one Fc fragment. Digested samples were purified using Poros R1 filter plate (Glyken corp.) and the Fc fragments were analysed for N-glycan site occupancy using MALDI-TOF MS (FIG. 2).

The overexpression of STT3 from Leishmania major enhanced the site coverage compared to the parental strain. The best clone was re-cultivated in three parallel shake flasks each and the analysis results were comparable to the first analysis. Compared to parental strain the signals Fc and Fc+K are practically absent in STT3 clones.

The difference in site occupancy between parental strain and all clones of STT3 from L. major was significant (FIG. 2). Because the signals coming from Fc or Fc+K were practically absent, the N-glycan site occupancy of MAB01 in these shake flask cultivations was 100% (Table 4).

TABLE 4
Site occupancy analysis of parental strain M317 and four
transformants of STT3 from L. major. The averages have
been calculated from area and intensity from single and
double charged signals from three parallel samples.

| | M317 | 17A-a | 26B-a | 65B-a | 97A-a |
|---|---|---|---|---|---|
| Glycosylation | Average | Average | Average | Average | Average |
| state | % | % | % | % | % |
| Non- | 13.0 | 0.0 | 0.0 | 0.0 | 0.0 |
| glycosylated | | | | | |
| Glycosylated | 87.0 | 100.0 | 100.0 | 100.0 | 100.0 |

Fermenter Cultivations

Three STT3 (L. major) clones (M420, M421 and M422) as well as parental strain M304 were cultivated in fermenter. Samples at day 3, 4, 5, 6 and 7 time points were collected and the site occupancy analysis was performed to purified antibody. STT3 overexpression strains and the respective control strain (M304) were grown in batch fermentations for 7 days, in media containing 2% yeast extract, 4% cellulose, 4% cellobiose, 2% sorbose, 5 g/L KH2PO4, and 5 g/L (NH4)2SO4. Culture pH was controlled at pH 5.5 (adjusted with NH₃OH). The temperature was shifted from 28° C. to 22° C. at 48 hours elapsed process time. Fermentations were carried out in 4 parallel 2 L glass vessel reactors with a culture volume of 1 L. Culture supernatant samples were taken during the course of the runs and stored at −20° C. MAB01 antibody was purified and digested with FabRICATOR as described above. The antibody titers are shown in Table 5.

Results

The site occupancy in parental strain M304 was less than 60% but in all analyzed STT3 clones the site occupancy had increased up to 98% (Table 6).

TABLE 5
MAB01 antibody titers of the LmSTT3 strains M420,
M421 and M422 and their parental strain M304.

| | Titer g/l | | | | |
|---|---|---|---|---|---|
| Strain | d3 | d4 | d5 | d6 | d7 |
| M304 | 0.225 | 0.507 | 0.981 | 1.52 | 1.7 |
| M420 | 0.758 | 1.21 | 1.55 | 1.71 | 1.69 |
| M421 | 0.76 | 1.24 | 1.54 | 1.67 | 1.6 |
| M422 | 0.65 | 1.07 | 1.43 | 1.56 | 1.54 |

TABLE 6
The N-glycosylation site occupancies of MAB01 antibody of the
LmSTT3 strains M420, M421 and M422 and their parental strain M304.

| | Site occupancy % | | | | |
|---|---|---|---|---|---|
| Strain | d3 | d4 | d5 | d6 | d7 |
| M304 | 48.0 | 47.7 | 47.7 | 46.3 | 55.4 |
| M420 | 97.8 | 97.5 | 96.9 | 94.3 | 94.6 |
| M421 | 96.1 | 90.8 | 91.5 | 89.7 | 95.6 |
| M422 | 94.4 | 88.5 | 80.9 | 83.6 | 75.2 |

In conclusion, overexpression of the STT3D gene from L. major increased the N-glycosylation site occupancy from 46%-87% in the parental strain to 98%-100% in transformants having Leishmania STT3 under shake flask or fermentation culture conditions.

The overexpression of the STT3D gene from L. major significantly increased the N-glycosylation site occupancy in strains producing an antibody as a heterologous protein. The antibody titers did not vary significantly between transformants having STT3 and parental strain.

### Example 2—Generation of T. reesei Strains Expressing STT3 from T. vaginalis, L. infantums or E. histolytica

The coding sequences of the Trichomonas vaginalis, Leishmania infantum and Entamoeba histolytica oligosaccharyl transferase (STT3; amino acid sequences T. vaginalis SEQ ID NO: 7, L. infantum SEQ ID NO: 8, and E. histolytica SEQ ID NO: 10) were codon optimized for Trichoderma reesei expression (codon optimized L. infantum nucleic acid SEQ ID NO: 9). The optimized coding sequences were synthesized along with T. reesei cbh1 terminator flanking sequence (SEQ ID NO: 11). Plasmids containing the STT3 genes under the constitutive cDNA1 promoter, with cbh1 terminator, pyr4 loopout marker and alg3 flanking regions (SEQ ID NO: 12 and SEQ ID NO: 13) were cloned by yeast homologous recombination as described in WO2012/069593. NotI fragment of plasmid pTTv38 was used as vector backbone. This vector contains alg3 (tre104121) 5′ and 3′ flanks of the gene to allow the expression cassette to integrate into the alg3 locus via homologous recombination in T. reesei and the plasmid has been described in WO2012/069593. The STT3 genes were excised from the cloning vectors using SfiI restriction enzyme digestion. The cdnaI promoter and cbh1 terminator fragments were created by PCR, using plasmids pTTv163 and pTTv166 as templates, respectively. The pyr4 loopout marker was extracted from plasmid pTTv142 by NotI digestion (the plasmid pTTv142 having a human GNT2 catalytic domain fused with T. reesei MNT1/KRE2 targeting peptide has been described in WO2012/069593). The pyr4 gene encodes the orotidine-5′-monophosphate (OMP) decarboxylase of T. reesei (Smith, J. L., et al., 1991, Current Genetics 19:27-33) and is needed for uridine synthesis. Strains deficient for OMP decarboxylase activity are unable to grow on minimal medium without uridine supplementation (i.e. are uridine auxotrophs). The primers used for cloning are listed in Table 7. The digested fragments and PCR products were separated with agarose gel electrophoresis and correct fragments were isolated from the gel with a gel extraction kit (Qiagen) according to manufacturer's protocol. The plasmids were constructed using the yeast homologous recombination method, using overlapping oligonucleotides for the recombination of the gap between the pyr4 marker and alg3 3′ flank as described in WO2012/069593. The plasmid DNA were rescued from yeast and transformed into electrocompetent TOP10 E. coli that were grown on ampicillin (100 μg/ml) selection plates. Miniprep plasmid preparations were made from several colonies. The presence of the Trichomonas vaginalis and Leishmania infantum STT3 genes was confirmed by digesting the prepared plasmids with BgIII-KpnI whereas the Entamoeba histolytica plasmid was digested with HindIII-KpnI. Positive clones were sequenced to verify the plasmid sequences. One correct Trichomonas vaginalis clone was chosen to be the final vector pTTv321, and correct clones of Leishmania infantum and Entamoeba histolytica were chosen to be the pTTv322 and pTTv323 vectors, respectively. The primers used for sequencing the vectors are listed in Table 8.

TABLE 7
List of primers used for cloning vectors pTTv321, pTTv322 and pTTv323.

| Fragment | Primer | Primer sequence |
|---|---|---|
| cDNA1 | T1177_pTTv321_1 | AGATTTCAGTCTCTCACCACTCACCTGAGTTGCCT |
| promoter, | | CTCTCGGTCTGAAGGACGTGGAATGATG |
| pTTv321 | | (SEQ ID NO: 54) |
| | T1178_pTTv321_2 | GCAGGGTGATGAGCTGGATCACCTTGACGGTGTT |
| | | GCCCATGTTGAGAGAAGTTGTTGGATTGATCA |
| | | (SEQ ID NO: 55) |
| cDNA1 | T1177_pTTv321_1 | AGATTTCAGTCTCTCACCACTCACCTGAGTTGCCT |
| promoter, | | CTCTCGGTCTGAAGGACGTGGAATGATG |
| pTTv322 | | (SEQ ID NO: 56) |
| | T1183_pTTv322_1 | CAGAGCCGCTATCGCCGAGGAGGTTGCCCTTCTT |
| | | GCCCATGTTGAGAGAAGTTGTTGGATTGATCA |
| | | (SEQ ID NO: 57) |
| cDNA1 | T1177_pTTv321_1 | AGATTTCAGTCTCTCACCACTCACCTGAGTTGCCT |
| promoter, | | CTCTCGGTCTGAAGGACGTGGAATGATG |
| pTTv323 | | (SEQ ID NO: 58) |
| | T1184_pTTv323_1 | TCTTGAGGATGAGCTGGACGAGGGTCTTGAAAAA |
| | | GCCCATGTTGAGAGAAGTTGTTGGATTGATCA |
| | | (SEQ ID NO: 59) |
| cbh1 | T1179_pTTv321_3 | AGCTCCGTGGCGAAAGCCTGA |
| terminator | | (SEQ ID NO: 60) |
| | T1180_pTTv321_4 | CAGCCGCAGCCTCAGCCTCTCTCAGCCTCATCAG |
| | | CCGCGGCCGCCAACTTTGCGTCCCTTGTGACG |
| | | (SEQ ID NO: 61) |
| pyr4-alg3 | T1181_pTTv321_5 | GCAACGAGAGCAGAGCAGCAGTAGTCGATGCTA |
| 3′ flank | | GGCGGCCGCGGGCAGTATGCCGGATGGCTGGCT |
| overlapping | | TATACAGGCA |
| oligos | | (SEQ ID NO: 62) |
| | T1182_pTTv321_6 | TGCCTGTATAAGCCAGCCATCCGGCATACTGCCC |
| | | GCGGCCGCCTAGCATCGACTACTGCTGCTCTGCT |
| | | CTCGTTGC |
| | | (SEQ ID NO: 63) |

TABLE 8
List of primers used for sequencing vectors
pTTv321, pTTv322 and pTTv323.

| Primer | Sequence |
|---|---|
| T027_Pyr4_orf_start_rev | TGCGTCGCCGTCTCGCTCCT |
| | (SEQ ID NO: 64) |
| T061_pyr4_orf_screen_2F | TTAGGCGACCTCTTTTTCCA |
| | (SEQ ID NO: 65) |
| T143_cDNA1promoter_seqF3 | CGAGGAAGTCTCGTGAGGAT |
| | (SEQ ID NO: 66) |
| T410_alg3_5-flank_F | CAGCTAAACCGACGGGCCA |
| | (SEQ ID NO: 67) |
| T1153_cbh1_term_start_rev | GACCGTATATTTGAAAAGGG |
| | (SEQ ID NO: 68) |

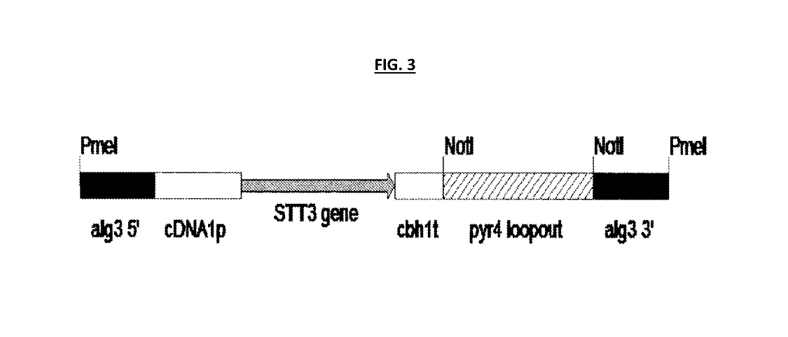

To prepare the vectors for transformation, the vectors were cut with PmeI to release the expression cassettes (FIG. 3). The fragments were separated with agarose gel electrophoresis and the correct fragment was isolated from the gel with a gel extraction kit (Qiagen) according to manufacturer's protocol. The purified expression cassette DNA was then transformed into protoplasts of the Trichoderma reesei M317. Preparation of protoplasts and transformation were carried out essentially according to methods in Penttila et al. (1987, Gene 61:155-164) and Gruber et al (1990, Curr. Genet. 18:71-76) for pyr4 selection. The transformed protoplasts were plated onto Trichoderma minimal media (TrMM) plates containing sorbitol.

Transformants were then streaked onto TrMM plates with 0.1% TritonX-100. Transformants growing fast as selective streaks were screened by PCR using the primers listed in Table 9. DNA from mycelia was purified and analyzed by PCR to look at the integration of the 5′ and 3′ flanks of cassette and the existence of the alg3 ORF. The cassette was targeted into the alg3 locus; therefore the open reading frame was not present in the positively integrated transformants, purified to single cell clones. To screen for 5′ integration, sequence outside of the 5′ integration flank was used to create a forward primer that would amplify genomic DNA flanking alg3 and the reverse primer was made from sequence in the cDNA1 promoter of the cassette. To check for proper integration of the cassette in the 3′ flank, a reverse primer was made from sequence outside of the 3′ integration flank that would amplify genomic DNA flanking alg3 and the forward primer was made from sequence in the pyr4 marker. Thus, one primer would amplify sequence from genomic DNA outside of the cassette and the other would amplify sequence from DNA in the cassette.

TABLE 9
List of primers used for PCR screening of T.
reesei transformants.

| 5′ flank screening primers: | 1165 bp product |
|---|---|
| T066_104121_5int | GATGTTGCGCCTGGGTTGAC |
| | (SEQ ID NO: 69) |
| T140_cDNA1promoter_seqR1 | TAACTTGTACGCTCTCAGTT |
| | CGA (SEQ ID NO: 70) |
| 3′ flank screening primers: | 1469 bp product |
| T026_Pyr4_orf_5rev2 | CCATGAGCTTGAACAGGTAA |
| | (SEQ ID NO: 71) |
| T068_104121_3int | GATTGTCATGGTGTACGTGA |
| | (SEQ ID NO: 72) |
| alg3 ORF primers: | 689 bp product |
| T767_alg3_del_F | CAAGATGGAGGGCGGCACAG |
| | (SEQ ID NO: 73) |
| T768_alg3_del_R | GCCAGTAGCGTGATAGAGAA |
| | GC (SEQ ID NO: 74) |
| alg3 ORF primers: | 1491 bp product |
| T069_104121_5orf_pcr | GCGTCACTCATCAAAACTGC |
| | (SEQ ID NO: 75) |
| T070_104121_3orf_pcr | CTTCGGCTTCGATGTTTCA |
| | (SEQ ID NO: 76) |

Four final strains each showing proper integration and a deletion of alg3 ORF were grown in large shake flasks in TrMM medium supplemented with 40 g/I lactose, 20 g/I spent grain extract, 9 g/I casamino acids and 100 mM PIPPS, pH 5.5. Growth for pTTv321 and pTTv323 strains was somewhat slower than parental strain M304 (Table 10). Three out of four Leishmania infantum pTTv322 clones grew somewhat better than the parental strain.

TABLE 10
Cell dry weight measurements (in g/L) of the parental
strains M304 and STT3 expressing strains.

| Strain | 3 days | 5 days | 7 days |
|---|---|---|---|
| M304 | 3.06 | 3.34 | 4.08 |
| pTTv321#18-9-2 | 2.54 | 2.89 | 2.52 |
| pTTv321#18-9-10 | 2.44 | 3.03 | 2.65 |
| pTTv321#18-12-1 | 2.43 | 3.12 | 2.86 |
| pTTv321#18-12-2 | 2.84 | 3.49 | 3.39 |
| pTTv322#60-2 | 3.02 | 3.42 | 3.63 |
| pTTv322#60-6 | 3.37 | 4.45 | 4.68 |
| pTTv322#60-12 | 3.30 | 4.15 | 4.29 |
| pTTv322#60-14 | 2.92 | 3.90 | 4.39 |
| pTTv323#37-4-1 | 2.29 | 2.27 | 2.59 |
| pTTv323#37-4-14 | 1.88 | 2.08 | 2.69 |
| pTTv323#37-11-3 | 2.15 | 2.27 | 2.62 |
| pTTv323#37-11-8 | 1.92 | 2.25 | 2.62 |

Site Occupancy and Glycan Analyses

From day 5 supernatant samples, MAB01 was purified using Protein G HP MultiTrap 96-well filter plate (GE Healthcare) according to manufacturer's instructions. Approx. 1.4 ml of culture supernatant was loaded and the elution volume was 230 μl. The antibody concentrations were determined via UV absorbance against MAB01 standard curve.

For site occupancy analysis 16-20 μg of purified MAB01 antibody was taken and antibodies were digested, purified, and analysed as described in example 1. The 100% site occupancy was achieved with Leishmania infantum STT3 clones 60-6, 60-12 and 60-14 (Table 11). In T. vaginalis and E. histolytica STT3 transformants the site occupancy was low and in the latter the antibodies appeared to be degraded resulting that no site occupancy analysis could be performed for one strain.

TABLE 11
N-glycosylation site occupancy of antibodies from STT3
variants and parental M304 at day 5.

| M304 | | | | |
|---|---|---|---|---|
| Glycosylation state | % | | | |
| Non-glycosylated | 8 | | | |
| Glycosylated | 92 | | | |
| Trichomonas vaginalis STT3, Δalg3 | 18-9-2 | 18-9-10 | 18-12-1 | 18-12-2 |
| Glycosylation state | % | % | % | % |
| Non-glycosylated | 75 | 71 | 69 | 64 |
| Glycosylated | 25 | 29 | 31 | 36 |
| Leishmania infantum STT3, Δalg3 | 60-2 | 60-6 | 60-12 | 60-14 |
| Glycosylation state | % | % | % | % |
| Non-glycosylated | 38 | 0 | 0 | 0 |
| Glycosylated | 62 | 100 | 100 | 100 |
| Entamoeba histolytica STT3, Δalg3 | 37-4-1 | 37-4-14 | 37-11-3 | 37-11-8 |
| Glycosylation state | % | % | % | % |
| Non-glycosylated | 82 | n.d. | 73 | 86 |
| Glycosylated | 18 | n.d. | 27 | 14 |

These results shows that overexpression of the catalytic subunit of Leishmania infantum is capable of increasing the N-glycosylation site occupancy in filamentous fungal cells, up to 100%.

In contrast, the STT3 genes from Trichomonas vaginalis or Entamoeba histolytica do not result in high N-glycosylation site occupancy.

N-glycans were analysed from three of the Leishmania infantum STT3 clones. The PNGase F reactions were carried out to 20 μg of MAB01 antibody as described in examples and the released N-glycans were analysed with MALDI-TOF MS. The three strains produced about 25% of Man3 N-glycan attached to MAB01 whereas Hex6 glycoform represents about 60% of N-glycans attached to MAB01 (Table 12).

TABLE 12
Neutral N-glycans and site occupancy analysis of
MAB01 from L. infantum STT3 clones at day 5.
Leishmania infantum STT3, Δalg3

| Clones | | 60-6 | 60-12 | 60-14 |
|---|---|---|---|---|
| Short | m\z | % | % | % |
| Man3 | 933.3 | 25.9 | 26.4 | 25.9 |
| Man4 | 1095.4 | 9.4 | 9.3 | 9.0 |
| Man5 | 1257.4 | 6.5 | 6.1 | 7.6 |
| Hex6 | 1419.5 | 58.3 | 58.2 | 57.5 |
| Fc | | 0 | 0 | 0 |
| Fc + Gn | | 0 | 0 | 0 |
| Glycosylated | | 100 | 100 | 100 |

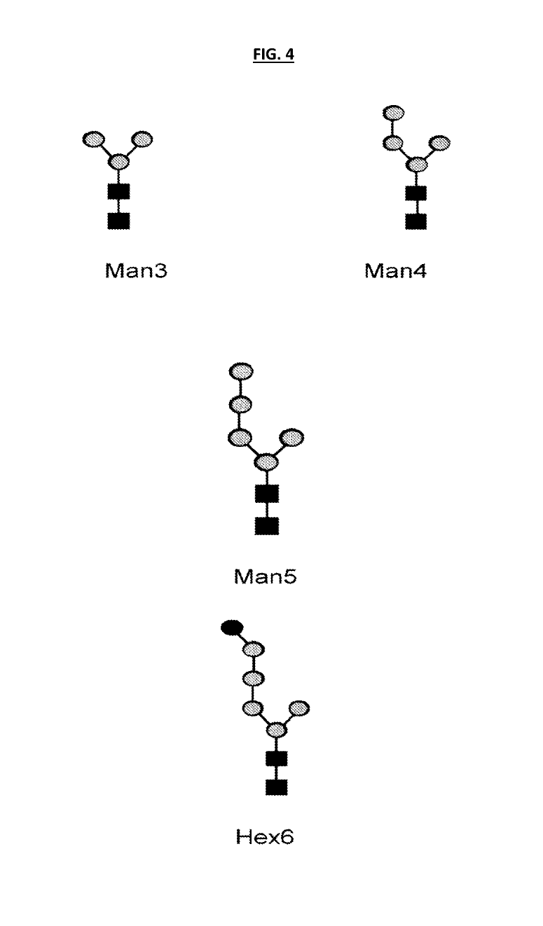

This shows that the Man3, G0, G1 and/or G2 glycoforms represent at least 25% of the total neutral N-glycans of MAB01 in 3 different clones overexpressing STT3 from L. infantum. FIG. 4 shows the glycan structures of Man3, Man4, Man5, and Hex6 produced in Δalg3 strains. “Fc” means an Fc fragment (without any N-glycans) and “Fc+Gn” means an Fc fragment with one attached N-acetylglucosamine (possible Endo T enzyme activity could cleave N-glycans of an Fc resulting Fc+Gn).

### Example 3—Generation of Δalg3 Strains of MAB01 Expressing Strains

The acetamide marker of the pTTv38 alg3 deletion plasmid was changed to pyr4 marker. The pTTv38 and pTTv142 vectors were digested with NotI and fragments separated with agarose gel electrophoresis. Correct fragments were isolated from the gel with a gel extraction kit (Qiagen) according to manufacturer's protocol. The purified pyr4 loopout marker from pTTv142 was ligated into the pTTv38 plasmid with T4 DNA ligase. The ligation reaction was transformed into electrocompetent TOP10 E. coli and grown on ampicillin (100 μg/ml) selection plates. Miniprep plasmid preparations were made from four colonies. The orientation of the marker was confirmed by sequencing the clones with primers listed in Table 13. A clone with the marker in inverted direction was chosen to be the final vector pTTv324.

TABLE 13
List of primers used for sequencing vectors
pTTv324.

| Primer | Sequence |
|---|---|
| T027_Pyr4_orf_start_rev | TGCGTCGCCGTCTCGCTCCT |
| | (SEQ ID NO: 77) |
| T060_pyr4_orf_screen_1F | TGACGTACCAGTTGGGATGA |
| | (SEQ ID NO: 78) |

A pyr4-strain of the Leishmania major STT3 expressing strain M420 was generated by looping out the pyr4 marker by 5-FOA selection as described in the International Patent Application No. PCT/EP2013/050126. One pyr4-strains was designated with number M602.

To prepare the vectors for transformation, the pTTv324 vector was cut with PmeI to release the deletion cassette. The fragments were separated with agarose gel electrophoresis and the correct fragment was isolated from the gel with a gel extraction kit (Qiagen) according to manufacturer's protocol. The purified deletion cassette DNA was then transformed into protoplasts of the Trichoderma reesei M317 and M602. Preparation of protoplasts, transformation, and protoplast plating were carried out as described above.

Transformants were then streaked onto TrMM plates with 0.1% TritonX-100. Transformants growing fast as selective streaks were screened by PCR using the primers listed in Table 14. DNA from mycelia was purified and analyzed by PCR to look at the integration of the 5′ and 3′ flanks of cassette and the existence of the alg3 ORF. The cassette was targeted into the alg3 locus; therefore the open reading frame was not present in the positively integrated transformants, purified to single cell clones. To screen for 5′ integration, sequence outside of the 5′ integration flank was used to create a forward primer that would amplify genomic DNA flanking alg3 and the reverse primer was made from sequence in the pyr4 marker of the cassette. To check for proper integration of the cassette in the 3′ flank, a reverse primer was made from sequence outside of the 3′ integration flank that would amplify genomic DNA flanking alg3 and the forward primer was made from sequence in the pyr4 marker. Thus, one primer would amplify sequence from genomic DNA outside of the cassette and the other would amplify sequence from DNA in the cassette.

TABLE 14
List of primers used for PCR screening of T.
reesei transformants.

| 5′ flank screening primers: | 1455 bp product |
|---|---|
| T066_104121_5int | GATGTTGCGCCTGGGTTGAC |
| | (SEQ ID NO: 79) |
| T060_pyr4_orf_screen_1F | TGACGTACCAGTTGGGATGA |
| | (SEQ ID NO: 80) |
| 3′ flank screening primers: | 1433 bp product |
| T027_Pyr4_orf_start_rev | TGCGTCGCCGTCTCGCTCCT |
| | (SEQ ID NO: 81) |
| T068_104121_3int | GATTGTCATGGTGTACGTGA |
| | (SEQ ID NO: 82) |
| alg3 ORF primers: | 689 bp product |
| T767_alg3_del_F | CAAGATGGAGGGCGGCACAG |
| | (SEQ ID NO: 83) |
| T768_alg3_del_R | GCCAGTAGCGTGATAGAGAA |
| | GC (SEQ ID NO: 84) |

Two M602 strains and seven M317 strains showing proper integration and a deletion of alg3 ORF were grown in large shake flasks in TrMM medium supplemented with 40 g/I lactose, 20 g/I spent grain extract, 9 g/I casamino acids and 100 mM PIPPS, pH 5.5 (Table 15). The M317 strain 19.13 and 19.20 were designated the numbers M697 and M698, respectively, and the M602 strains 1.22 and 11.18 were designated the numbers M699 and M700, respectively.

TABLE 15
Cell dry weight measurements (in g/l) of the parental strains M304 and
STT3 expressing strain M420 and alg3 deletion transformants.

| Strain | 3 days | 5 days | 7 days |
|---|---|---|---|
| M602 1.22 | 3.63 | 3.23 | 3.79 |
| M602 11.18 | 3.52 | 3.74 | 4.12 |
| M317 19.1 | 3.64 | 3.84 | 4.22 |
| M317 19.5 | 3.54 | 3.87 | 4.31 |
| M317 19.6 | 3.72 | 3.66 | 4.78 |
| M317 19.13 | 3.63 | 3.21 | 4.06 |
| M317 19.20 | 3.97 | 4.28 | 5.09 |
| M317 19.43 | 3.77 | 4.02 | 4.18 |
| M317 19.44 | 3.58 | 3.78 | 4.17 |
| M420 | 3.31 | 3.69 | 5.57 |
| M304 | 2.55 | 2.99 | 4.09 |

Site Occupancy and Glycan Analyses

Two transformants from overexpression of STT3 from Leishmania major in alg3 deletion strain [pTTv324; 1.22 (M699) and 11.18 (M700)] and seven transformants with alg3 deletion [M317, pyr4- of M304; clones 19.1, 19.5, 19.6, 19.13 (M697), 19.20 (M698), 19.43 and 19.44], and their parental strains M420 and M304 were cultivated in shake flasks in TrMM, 4% lactose, 2% spent grain extract, 0.9% casamino acids, 100 mM PIPPS, pH 5.5. MAB01 antibody was purified and analysed from culture supernatants from day 5 as described in Example 1 except that 30 μg of antibody was digested with 80.4 U of FabRICATOR (Genovis), +37° C., overnight, to produce F(ab′)2 and Fc fragments.

In both clones with alg3 deletion and overexpression of LmSTT3 the site occupancy was 100% (Table 16). Without LmSTT3 the site coverage varied between 56-71% in alg3 deletion clones. The improved site occupancy was shown also in parental strain M420 compared to M304, both with wild type glycosylation.

TABLE 16
The site occupancy of the shake flask samples. The
analysis failed in M317 clones 19.5 and 19.6.

| | | | Site |
|---|---|---|---|
| Strain | Clone | Explanation | occupancy % |
| M602 | 1.22 | M304 LmSTT3 Δalg3 | 100 |
| M602 | 11.18 | M304 LmSTT3 Δalg3 | 100 |
| M317 | 19.1 | M304 Δalg3 | 71 |
| M317 | 19.13 | M304 Δalg3 | 62 |
| M317 | 19.2 | M304 Δalg3 | 56 |
| M317 | 19.43 | M304 Δalg3 | 63 |
| M317 | 19.44 | M304 Δalg3 | 60 |
| M420 | | Parental strain M304 | 100 |
| | | LmSTT3 | |
| M304 | | Parental strain | 89 |

For N-glycan analysis MAB01 was purified from day 7 culture supernatants as described above and N-glycans were released from EtOH precipitated and SDS denatured antibody using PNGase F (Prozyme) in 20 mM sodium phosphate buffer, pH 7.3, in overnight reaction at +37° C. The released N-glycans were purified with Hypersep C18 and Hypersep Hypercarb (Thermo Scientific) and analysed with MALDI-TOF MS.

Man3 levels were in range of 21 to 49% whereas the main glycoform in clones of M602 and M317 was Hex6 (Table 17). Man5 levels were about 73% in the strains expressing wild type glycosylation (M304) and LmSTT3 (M420).

TABLE 17
Relative proportions of neutral N-glycans from purified antibody from M602 and
M317 clones and parental strains M420 and M304.

| | | | M602 | | M317 | | | | | Parental strains | |
|---|---|---|---|---|---|---|---|---|---|---|---|
| | | | 1.22 | 11.18 | 19.1 | 19.13 | 19.2 | 19.43 | 19.44 | M420 | M304 |
| Composition | Short | m\z | % | % | % | % | % | % | % | % | % |
| Hex3HexNAc2 | Man3 | 933.3 | 21.1 | 27.3 | 45.4 | 37.5 | 34.9 | 24.6 | 48.6 | 0.0 | 0.0 |
| Hex4HexNAc2 | Man4 | 1095.4 | 9.5 | 8.7 | 6.2 | 7.6 | 7.1 | 7.5 | 9.4 | 0.8 | 0.0 |
| Hex5HexNAc2 | Man5 | 1257.4 | 5.8 | 7.0 | 8.1 | 7.6 | 6.7 | 5.6 | 6.6 | 72.5 | 72.8 |
| Hex6HexNAc2 | Man6/Hex6 | 1419.5 | 63.1 | 56.6 | 39.7 | 45.8 | 51.4 | 61.8 | 34.6 | 15.6 | 16.4 |
| Hex7HexNAc2 | Man7/Hex7 | 1581.5 | 0.5 | 0.5 | 0.6 | 0.8 | 0.0 | 0.5 | 0.7 | 7.2 | 7.9 |
| Hex8HexNAc2 | Man8/Hex8 | 1743.6 | 0.0 | 0.0 | 0.0 | 0.6 | 0.0 | 0.0 | 0.0 | 3.2 | 2.4 |
| Hex9HexNAc2 | Man9/Hex9 | 1905.6 | 0.0 | 0.0 | 0.0 | 0.0 | 0.0 | 0.0 | 0.0 | 0.7 | 0.5 |

Fermentation and Site Occupancy

L. major STT3 alg3 deletion strain M699 (pTTv324; clone 1.22) and strain M698 with alg3 deletion [M317, pyr4- of M304; clone 19.20], and the parental strain M304 were fermented in 2% YE, 4% cellulose, 8% cellobiose, 4% sorbose. The samples were harvested on day 3, 4, 5 and 6. MAB01 antibody was purified and analysed from culture supernatants from day 5 as described in Example 1 except that 30 μg of antibody was digested with 80.4 U of FabRICATOR (Genovis), +37° C., overnight, to produce F(ab′)2 and Fc fragments.

Results

In the strain M699 site occupancy was more than 90% in all time points (Table 18). Without LmSTT3 the site coverage varied between 29-37% in the strain M698. In the parental strain M304 the site coverage varied between 45-57%. At day 6 MAB01 titers were 1.2 and 1.3 g/L for strains M699 and M698, respectively, and 1.8 g/L in the parental strain M304.

TABLE 18
MAB01 antibody titers and site occupancy
analysis results of fermented strains M699
and M698 and the parental strain M304.

| | d3 | d4 | d5 | d6 |
|---|---|---|---|---|
| M699 | | | | |
| Titer g/l | 0.206 | 0.361 | 0.685 | 1.22 |
| Glycosylation state | % | % | % | % |
| Non-glycosylated | 2.4 | 6.8 | 8.0 | 8.5 |
| Glycosylated | 97.6 | 93.2 | 92.0 | 91.5 |
| Fc + Gn | 0.0 | 0.0 | 0.0 | 0.0 |
| M698 | | | | |
| Titer g/l | 0.252 | 0.423 | 0.8 | 1.317 |
| Glycosylation state | % | % | % | % |
| Non-glycosylated | 63.0 | 70.8 | 64.3 | 65.8 |
| Glycosylated | 37.0 | 29.2 | 35.7 | 34.2 |
| Fc + Gn | 0.0 | 0.0 | 0.0 | 0.0 |
| M304 | | | | |
| Titer g/l | 0.589 | 0.964 | 1.41 | 1.79 |
| Glycosylation state | % | % | % | % |
| Non-glycosylated | 45.9 | 43.3 | n.d. | 54.9 |
| Glycosylated | 54.1 | 56.7 | n.d. | 45.1 |
| Fc + Gn | 0.0 | 0.0 | n.d. | 0.0 |

In conclusion, overexpression of the catalytic subunit of Leishmania STT3 is capable of increasing the N-glycosylation site occupancy in Δalg3 filamentous fungal cells up to 91.5-100%.

Table 19 below recapitulates the different strains used in the Examples:

| | | | | Locus, | | | | |
|---|---|---|---|---|---|---|---|---|
| | | | Strain | random or | | | Selection | |
| Database | Vector | Clone | transformed | K/o | Proteases k/o | Description | of tr. | Markers in strain |
| M44 | | | | | None | Base strain | | None |
| M124 | | | | K/o mus53 | None | mus53 deletion of M44 | | pyr4 |
| M127 | | | | pyr4− of | None | pyr4 negative strain of | | pyr4− |
| | | | | M124 | | M124 | | |
| M181 | pTTv71 | 9-20A- | M127 | K/o pep1 | pep1 | pep1 deletion | pyr4 | pyr4 |
| | | 1 | | | | | | |
| M194 | pTTv42 | 13- | M181 | K/o tsp1 | pep1 tsp1 | pep1 tsp1 deletion | bar | bar/pyr4 |
| | | 172D | | | | | | |
| M252 | pTTv99/67 | 6.14A | M194 | cbh1 egl1 | pep1 tsp1 | MAB01 LC NVISKR/HC | AmdS/ | AmdS/HygR/bar/pyr4 |
| | | | | loci | | AXE1 | HygR | |
| M284 | 5-FOA of | 3A | pyr4− of | Spontaneous | pep1 tsp1 | pyr4 negative strain of | none | AmdS/HygR/bar/pyr4− |
| | M252 | | M252 | mutation | | M252 | | |
| M304 | pTTv128 | 12A | M284 | K/o slp1, | pep1 tsp1 slp1 | Overexpression of native | pyr4 | AmdS/HygR/bar/pyr4 |
| | | | | Kex2 o/e | | Kex2, slp1 del | | |
| M317 | 5-FOA of | pyr4− | M304 | pyr4 loopout | pep1 tsp1 slp1 | pyr4 negative strain of | None | AmdS/HygR/bar/pyr4− |
| | M304 | of 1A | | | | M304 | | |
| M420 | pTTv201 | 17A-a | M317 | xylanase 1 | pep1 tsp1 slp1 | Leishmania major stt3 | pyr4 | AmdS/HygR/bar/pyr4 |
| | | | | | | Oligosaccharyl transferase | | |
| M421 | pTTv201 | 26B-a | M317 | xylanase 1 | pep1 tsp1 slp1 | Leishmania major stt3 | pyr4 | AmdS/HygR/bar/pyr4 |
| | | | | | | Oligosaccharyl transferase | | |
| M422 | pTTv201 | 65B-a | M317 | xylanase 1 | pep1 tsp1 slp1 | Leishmania major stt3 | pyr4 | AmdS/HygR/bar/pyr4 |
| | | | | | | Oligosaccharyl transferase | | |
| M423 | pTTv201 | 97A-a | M317 | xylanase 1 | pep1 tsp1 slp1 | Leishmania major stt3 | pyr4 | AmdS/HygR/bar/pyr4 |
| | | | | | | Oligosaccharyl transferase | | |
| M602 | 5-FOA of | pyr4− | M420 | pyr4 loopout | pep1 tsp1 slp1 | pyr4 negative strain of | none | AmdS/HygR/bar/pyr4− |
| | M420 | of 2A | | | | M420 | | |
| M698 | pTTv324 | 19.20 | M317 | alg3 | pep1 tsp1 slp1 | Deletion of alg3 | pyr4 | AmdS/HygR/bar/pyr4 |
| M699 | pTTv324 | 1.22 | M602 | alg3 | pep1 tsp1 slp1 | Deletion of alg3 | pyr4 | AmdS/HygR/bar/pyr4 |
| M800 | pTTv322 | 60-6 | M317 | alg3 | pep1 tsp1 slp1 | Leishmania infantum STT3, | pyr4 | AmdS/HygR/bar/pyr4 |
| | | | | | | cDNA1p cbh1t | | |
| M801 | pTTv322 | 60-12 | M317 | alg3 | pep1 tsp1 slp1 | Leishmania infantum STT3, | pyr4 | AmdS/HygR/bar/pyr4 |
| | | | | | | cDNA1p cbh1t | | |
| M802 | pTTv322 | 60-14 | M317 | alg3 | pep1 tsp1 slp1 | Leishmania infantum STT3, | pyr4 | AmdS/HygR/bar/pyr4 |
| | | | | | | cDNA1p cbh1t | | |

Trichoderma strains having STT3 (M420-M423) are triple protease deficient (pep1, tsp1, slp1) as well as deficient of xylanase1, cbh1, and egl1. Embodiments include also higher order protease deficient strains.

1-15. (canceled)

16. A filamentous fungal cell comprising
i. one or more mutations that reduces or eliminates one or more endogenous protease activity compared to a parental filamentous fungal cell which does not have said mutation(s),
ii. a polynucleotide encoding a heterologous catalytic subunit of oligosaccharyl transferase, and
iii. a polynucleotide encoding a heterologous glycoprotein,
wherein said catalytic subunit of oligosaccharyl transferase is selected from Leishmania oligosaccharyl transferase catalytic subunits.

17. The filamentous fungal cell of claim 16, wherein the filamentous fungal cell is a Trichoderma, Neurospora, Myceliophtora, Chrysosporium, Aspergillus, or Fusarium cell.

18. The filamentous fungal cell of claim 16, wherein said polynucleotide encoding the heterologous catalytic subunit of oliogaccharyl transferase comprises a nucleic acid selected from the group consisting of SEQ ID NO: 2, SEQ ID NO: 9, SEQ ID NO: 88 and SEQ ID NO: 90 or a polynucleotide encoding a functional variant polypeptide having at least 50%, at least 60%, at least 70% identity, at least 80% identity, at least 90% identity, or at least 95% identity with SEQ ID NO: 1, SEQ ID NO: 8, SEQ ID NO: 89 or SEQ ID NO:91, said functional variant polypeptide having oligosaccharyltransferase activity.

19. The filamentous fungal cell of claim 16, wherein the N-glycosylation site occupancy of the heterologous glycoprotein is at least 95% and Man3, Man5, G0, G1 and/or G2 glycoforms represent at least 50% of total neutral N-glycans of the heterologous glycoprotein.

20. The filamentous fungal cell of claim 16, wherein said cell is a Trichoderma cell and said cell comprises mutations that reduce or eliminate the activity of
the three endogenous proteases pep1, tsp1, and slp1;
the three endogenous proteases gap1, slp1, and pep1;
the three endogenous proteases selected from the group consisting of pep1, pep2, pep3, pep4, pep5, pep8, pep9, pep11, pep12, tsp1, slp1, slp2, slp3, slp7, gap1 and gap2;
three to six proteases selected from the group consisting of pep1, pep2, pep3, pep4, pep5, tsp1, slp1, slp2, slp3, gap1 and gap2; or,
seven to ten proteases selected from the group consisting of pep1, pep2, pep3, pep4, pep5, pep7, pep8, tsp1, slp1, slp2, slp3, slp5, slp6, slp7, slp8, tpp1, gap1 and gap2.

21. The filamentous fungal cell of claim 16, wherein the fungal cell further comprises a mutation in the gene encoding ALG3 that reduces or eliminates the corresponding ALG3 expression compared to the level of expression of ALG3 gene in a parental cell which does not have such mutation.

22. The filamentous fungal cell of claim 16, further comprising a polynucleotide encoding an N-acetylglucosaminyltransferase I catalytic domain and an N-acetylglucosaminyltransferase II catalytic domain.

23. The filamentous fungal cell of claim 16, further comprising one or more polynucleotides encoding a polypeptide selected from the group consisting of:
i. α1, 2 mannosidase;
ii. N-acetylglucosaminyltransferase I catalytic domain;
iii. α-mannosidase II;
iv. N-acetylglucosaminyltransferase II catalytic domain;
v. β1,4 galactosyltransferase; and,
vi. fucosyltransferase.

24. A method of producing a heterologous glycoprotein, or antibody composition, with increased N-glycosylation site occupancy, comprising
a) providing a filamentous fungal cell having a Leishmania STT3D gene encoding a catalytic subunit of oligosaccharyl transferase, or a functional variant thereof, and a polynucleotide encoding said heterologous glycoprotein or antibody,
b) culturing the cell under appropriate conditions for expression of the STT3D gene or its functional variant, or said functional variant, and the production of the heterologous glycoprotein; and,
c) recovering and, optionally, purifying the heterologous glycoprotein.

25. The method of claim 24, wherein said filamentous fungal host cell comprises one or more mutation(s) that reduces or eliminates one or more endogenous protease activity compared to a parental filamentous fungal cell which does not have said mutation.

26. The method of claim 24, wherein said filamentous fungal host cell comprises:
i. one or more mutations that reduces or eliminates one or more endogenous protease activity compared to a parental filamentous fungal cell which does not have said mutation(s),
ii. a polynucleotide encoding a heterologous catalytic subunit of oligosaccharyl transferase selected from Leishmania oligosaccharyl transferase catalytic subunits, and
iii. a polynucleotide encoding a heterologous glycoprotein.

27. The method of claim 24, wherein said Leishmania STT3D gene encoding a catalytic subunit of oligosaccharyl transferase comprises a nucleic acid sequence selected from the group consisting of SEQ ID NO:2, SEQ ID NO:9, SEQ ID NO: 88 and SEQ ID NO: 90, or a polynucleotide encoding a functional variant polypeptide having at least 50%, at least 60%, at least 70% identity, at least 80% identity, at least 90% identity, or at least 95% identity with SEQ ID NO:1, SEQ ID NO:8, SEQ ID NO: 89 or SEQ ID NO: 91, said functional variant polypeptide having oligosaccharyltransferase activity.

28. The method of claim 24, wherein N-glycosylation site occupancy of the produced glycoprotein composition is at least 80%.

29. A glycoprotein or antibody composition obtainable by the method of claim 24.

30. The glycoprotein or antibody composition according to claim 29, wherein said antibody composition further comprises, as a major glycoform, either:
i. Manα3[Manα6(Manα3)Manα6]Man134GlcNA134GlcNAc (Man5 glycoform);
ii. GlcNAcβ2Manα3 [Manα6(Manα3)Manα6]Manβ4GlcNAβ4GlcNAc (GlcNAcMan5 glycoform);
iii. Manα6(Manα3)Manβ4GlcNAβ4GlcNAc (Man3 glycoform);
iv. Manα6(GlcNAcβ2Manα3)Manβ4GlcNAβ4GlcNAc (GlcNAcMan3 glycoform); or,
v. complex type N-glycans selected from the G0, G1, and G2 glycoforms.
